SEQUENCE LISTING (17 sequences)

SEQ ID NO: 1 - RNA, 19 nt, Artificial Sequence
  Description of Artificial Sequence Synthetic oligonucleotide
ugguuuacaugucgacuaa

SEQ ID NO: 2 - RNA, 19 nt, Artificial Sequence
  Description of Artificial Sequence Synthetic oligonucleotide
ugguuuacauguuguguga

SEQ ID NO: 3 - RNA, 19 nt, Artificial Sequence
  Description of Artificial Sequence Synthetic oligonucleotide
ugguuuacauguuuucuga

SEQ ID NO: 4 - RNA, 19 nt, Artificial Sequence
  Description of Artificial Sequence Synthetic oligonucleotide
ugguuuacauguuuuccua

SEQ ID NO: 5 - RNA, 21 nt, Artificial Sequence
  Description of Artificial Sequence Synthetic oligonucleotide
uuccuuuagacccauucucuu

SEQ ID NO: 6 - RNA, 19 nt, Artificial Sequence
  Description of Artificial Sequence Synthetic oligonucleotide
gaacccaccacugcuucuc

SEQ ID NO: 7 - DNA, 20 bp, Artificial Sequence
  Description of Artificial Sequence Synthetic primer
agcttcggagaagcagtggt

SEQ ID NO: 8 - DNA, 18 bp, Artificial Sequence
  Description of Artificial Sequence Synthetic primer
ttctttccgcggagacct

SEQ ID NO: 9 - DNA, 19 bp, Artificial Sequence
  Description of Artificial Sequence Synthetic primer
acagatgaaccgcggagac

SEQ ID NO: 10 - DNA, 20 bp, Artificial Sequence
  Description of Artificial Sequence Synthetic primer
taagggtcatggagctggag

SEQ ID NO: 11 - DNA, 20 bp, Artificial Sequence
  Description of Artificial Sequence Synthetic primer
atcagctcctccccgagtat

SEQ ID NO: 12 - DNA, 22 bp, Artificial Sequence
  Description of Artificial Sequence Synthetic primer
gaagtttaatgttgccaatgga

SEQ ID NO: 13 - DNA, 20 bp, Artificial Sequence
  Description of Artificial Sequence Synthetic primer
gcctttgcacagactgacct

SEQ ID NO: 14 - DNA, 20 bp, Artificial Sequence
  Description of Artificial Sequence Synthetic primer
atccagaactgcagccagtc

SEQ ID NO: 15 - DNA, 20 bp, Artificial Sequence
  Description of Artificial Sequence Synthetic primer
agaagtacatgggggtgtgg

SEQ ID NO: 16 - RNA, 21 nt, Artificial Sequence
  Description of Artificial Sequence Synthetic oligonucleotide
aagagaaugggucuaaaggaa

SEQ ID NO: 17 - RNA, 19 nt, Artificial Sequence
  Description of Artificial Sequence Synthetic oligonucleotide
gagaagcagugguggguuc

(19) United States
(12) Patent Application Publication
(10) Pub. No.: US 2019/0249257 A1
(43) Pub. Date: Aug. 15, 2019

(54) LONG INTERGENIC NON-CODING RNA AS PANCANCER BIOMARKER

(71) Applicant: UNIVERSITY OF HAWAII, Honolulu, HI (US)

(72) Inventors: Travers CHING, Honolulu, HI (US); Lana GARMIRE, Honolulu, HI (US)

(73) Assignee: UNIVERSITY OF HAWAII, Honolulu, HI (US)

(21) Appl. No.: 16/079,490

(22) PCT Filed: Feb. 27, 2017
(86) PCT No.: PCT/US2017/019730

§ 371 Date: Aug. 23, 2018

Related U.S. Application Data
(60) Provisional application No. 62/300,614, filed on Feb. 26, 2016.

(51) Int. Cl. C12Q 1/6886

(52) U.S. Cl. CPC C12Q 1/6886; C12Q 2600/158; C12Q 2600/118

(57) ABSTRACT

Certain embodiments of the invention provide a method for identifying a cancer cell, comprising detecting increased Sexpression of at least one lincRNA selected from the group consisting of PCAN-1, PCAN-2, PCAN-3, PCAN-5 and PCAN-6 and/or decreased expression of lincRNA PCAN-4 in a nucleic acid sample derived from the cell, wherein increased expression of at least one of PCAN-1, PCAN-2, PCAN-3, PCAN-5 and PCAN-6 and/or decreased expression of PCAN-4, as compared to expression from a control cell, indicates the cell is a cancer cell.

### RELATED APPLICATION

This application claims the benefit of priority of U.S. Provisional Application Ser. No. 62/300,614 filed on Feb. 26, 2016, which application is incorporated by reference herein.

### GOVERNMENT FUNDING

This invention was made with government support under R01 LM012373, R01 HD084633, P20 GM103457, and K01 ES025434 awarded by the National Institutes of Health. The government has certain rights in the invention.

### BACKGROUND

Throughout the world, cancer is among the leading causes of death. In 2012, there were 14 million new cases and 8.2 million cancer-related deaths worldwide. The number of new cancer cases is expected rise to 22 million within the next two decades. Effectively treating cancer often depends on early detection and the ability to accurately monitor therapy. In many cancers, protein-coding genes have altered expression; however, these changes often do not have the requisite specificity or are undetectable by current methods. Further, the epigenetic states of human cancers, such as chromatin modification of specific genes, are difficult to measure in patient samples. Thus, there remains a need for new methods to diagnose cancer, monitor therapy and predict cancer prognosis.

Thus, there is a need to identify new biomarkers that are associated with cancer. In particular, there is a need to identify new biomarkers, which may be used for diagnositic tests and/or prognostic indices.

### SUMMARY

Accordingly, described herein is the identification of pan-cancer lincRNA biomarkers, which may be used, e.g., as a screening (e.g., for pan-cancer), diagnositic and/or prognositc tool for multiple cancer types. As described herein, this panel of biomarkers may be used to simultaneously diagnose multiple cancer types and has been shown to accurately predict cancer vs. non-cancer tissue types for breast, head and neck, thyroid, colon, kidney, liver, lung, prostate, gastric, and endometrial cancers (see, Example 1).

Thus, certain embodiments of the invention provide a method for identifying a cancer cell, comprising detecting increased expression of at least one lincRNA selected from the group consisting of PCAN-1, PCAN-2, PCAN-3, PCAN-5 and PCAN-6 and/or decreased expression of lincRNA PCAN-4 in a nucleic acid sample derived from the cell, wherein increased expression of at least one of PCAN-1, PCAN-2, PCAN-3, PCAN-5 and PCAN-6 and/or decreased expression of PCAN-4 as compared to expression from a control cell indicates the cell is a cancer cell.

Certain embodiments of the invention provide a method for identifying a patient having cancer, comprising detecting increased expression of at least one lincRNA selected from the group consisting of PCAN-1, PCAN-2, PCAN-3, PCAN-5 and PCAN-6 and/or decreased expression of lincRNA PCAN-4 in a nucleic acid sample that was derived from a biological sample obtained from the patient, wherein increased expression of at least one of PCAN-1, PCAN-2, PCAN-3, PCAN-5 and PCAN-6 and/or decreased expression of PCAN-4, as compared to expression from a control sample, indicates the patient has cancer.

Certain embodiments of the invention provide a method for establishing a prognosis for a patient having cancer, comprising:

1) detecting the expression levels of at least one lincRNA selected from the group consisting of PCAN-1, PCAN-2, PCAN-3, PCAN-4, PCAN-5 and PCAN-6 in a nucleic acid sample derived from biological sample that was obtained from the patient; and

2) comparing the expression levels to a relative cut-off value, wherein expression levels that are higher than the relative cut-off level for PCAN-1, PCAN-2, PCAN-3, PCAN-5 and/or PCAN-6 are indicative of a poor prognosis, and wherein expression levels that are lower than a relative cut-off level for PCAN-4 are indicative of a poor prognosis.

Certain embodiments of the invention provide a method comprising:

1) detecting increased expression of at least one lincRNA selected from the group consisting of PCAN-1, PCAN-2, PCAN-3, PCAN-5 and PCAN-6 and/or decreased expression of lincRNA PCAN-4 in a nucleic acid sample that was derived from a biological sample obtained from a patient;

2) diagnosing the patient with cancer when increased expression of at least one lincRNA selected from the group consisting of PCAN-1, PCAN-2, PCAN-3, PCAN-5 and PCAN-6 is detected and/or decreased expression of lincRNA PCAN-4 is detected, as compared to expression from a control sample; and

3) administering an effective amount of a therapeutic agent to the patient.

Certain embodiments of the invention provide a method for treating cancer in a patient comprising administering an effective amount of a therapeutic agent to the patient, wherein the cancer was determined to comprise increased expression of at least one lincRNA selected from the group consisting of PCAN-1, PCAN-2, PCAN-3, PCAN-5 and PCAN-6 and/or decreased expression of lincRNA PCAN-4, as compared to expression from a control.

Certain embodiments of the invention provide a therapeutic agent for the prophylactic or therapeutic treatment of a cancer determined to comprise increased expression of at least one lincRNA selected from the group consisting of PCAN-1, PCAN-2, PCAN-3, PCAN-5 and PCAN-6 and/or decreased expression of lincRNA PCAN-4, as compared to expression from a control.

Certain embodiments of the invention provide the use of a therapeutic agent to prepare a medicament for treating cancer in a patient determined to comprise increased expression of at least one lincRNA selected from the group consisting of PCAN-1, PCAN-2, PCAN-3, PCAN-5 and PCAN-6 and/or decreased expression of lincRNA PCAN-4, as compared to expression from a control.

Certain embodiments of the invention provide a method for identifying an effective cancer treatment in a patient, comprising:

1) detecting the expression level of at least one lincRNA selected from the group consisting of PCAN-1, PCAN-2, PCAN-3, PCAN-4, PCAN-5 and PCAN-6 in a first biological sample obtained from the patient before the cancer treatment;

2) detecting the expression level of at least one lincRNA selected from the group consisting of PCAN-1, PCAN-2, PCAN-3, PCAN-4, PCAN-5 and PCAN-6 in a second biological sample obtained from the patient after the cancer treatment; and

3) identifying the cancer treatment as effective based on the level of lincRNA expression, wherein decreased expression of at least one of PCAN-1, PCAN-2, PCAN-3, PCAN-5 or PCAN-6 in the second sample as compared to the first sample and/or increased expression of PCAN-4 in the second sample as compared to the first sample indicates that the cancer treatment is effective.

Certain embodiments of the invention provide a kit comprising:

1) at least one reagent for detecting increased expression of at least one lincRNA selected from the group consisting of PCAN-1, PCAN-2, PCAN-3, PCAN-5 and PCAN-6 and/or decreased expression of lincRNA PCAN-4 in a cell; and

2) instructions for using the reagent, wherein increased expression of at least one of PCAN-1, PCAN-2, PCAN-3, PCAN-5 and PCAN-6 and/or decreased expression of PCAN-4, as compared to expression from a control cell, indicates the cell is a cancer cell.

### BRIEF DESCRIPTION OF THE FIGURES

FIGS. 1A-B. Principal component analysis of lincRNA expression in 12 TCGA datasets. The first three principal components (PCs) were plotted using the log FPKM values of lincRNA expression in (FIG. 1A) normal adjacent tissue and (FIG. 1B) cancer samples. The variances associated with each of the first 10 principal components are plotted alongside each graph (Scree Plot).

FIGS. 2A-F. Tissue specificities of lincRNAs and protein coding genes. (FIGS. 2A-B) Maximum JS scores were used to measure tissue specificity in primary tumors and adjacent normal samples, based on either lincRNAs (FIG. 2A) or protein coding genes (FIG. 2B). A value of 1 indicates that the lincRNA is expressed in only one tissue. (FIGS. 2C-F) Fractional expression of lincRNAs or protein coding genes in each tissue was plotted in the adjacent normal or cancer samples.

FIGS. 3A-C. Correlation of lincRNAs with other data types and cancer subtypes. (FIG. 3A) The concordance between clustering results of lincRNAs and other high throughput data types in TCGA based on χ-square statistical test. (FIG. 3B) CNMF was used to determine the clustering of lincRNAs in the GSE58135 Breast Cancer dataset. The concordance of the clustering with the tumor subtypes in the dataset is significant (chi-square, p<2.2e-16). (FIG. 3C) CNMF was used to determine the clustering of lincRNA in the TCGA BRCA dataset. The concordance of the CNMF clustering with the tumor subtypes in the dataset is significant (chi-square, p<2.2e-16).

FIGS. 4A-D. Differentially expressed pan-cancer lincRNAs. (FIG. 4A) Six lincRNAs are consistently differentially expressed in 12 TCGA datasets. Each of the six lincRNAs shown is either significantly upregulated or significantly downregulated across the various cancers. The six lincRNAs in three independent RNA-Seq datasets from GEO (FIG. 4B), our own breast cancer dataset (FIG. 4C) and qPCR of pooled 5 normal tissues, pooled 5 tumors and the MCF-7 cell line (FIG. 4D).

FIGS. 5A-D. The pan-cancer diagnostic model for the lincRNA panel. (FIG. 5A) The classification of the lincRNA panel was based on a computational RNA-Seq pipeline. The TCGA data were split into 80% training and 20% testing subsets. Five out of the six lincRNAs were selected as predictive features using Correlation Feature Selection (CFS). Pan-cancer diagnostic models were constructed using four standard classification machine learning methods: Random Forest (RF), Linear Support Vector Machines (LSVM), Gaussian Support Vector Machines (GSVM) and Logistic Regression (L2-LR). The best model was chosen based on various metrics of the Receiver operating characteristic (ROC) curves, including Area Under the Curve (AUC), F-score, Matthew's correlation coefficient (MCC) and Accuracy. (FIG. 5B) The performance of the classifier was analyzed with the ROC curves on the TCGA hold-out testing data, based on the four classification methods mentioned above and (FIG. 5C) ROC curves of the top Random Forest model on four independent RNA-Seq validation datasets. (FIG. 5D) AUCs were calculated on the TCGA hold-out testing data and the four validation datasets.

FIGS. 6A-D. The effect of lincRNAs downregulation on cell proliferation and migration. (FIG. 6A) XLOC_12_004121 and XLOC_12_004340 lincRNAs can be efficiently knocked down in MDA-MB-231 and MCF-7 cell lines. Bars represent RT-qPCR results of XLOC_12_004121 (linc4121) and XLOC_12_004340 (linc4340) expression. siRNA lincRNA bars show mean expression (n=3) with S. D. normalized to the control condition. (FIG. 6B) Transient knockdown of XLOC_12_004121 and XLOC_12_004340 inhibits the growth rate of MDA-MB-231 cells. 30 hours after transfection (time point “0”) 400 cells were seeded in 96-well plates and processed for luminescent cell viability assay at indicated time points. Data points represent mean value (n=3), error bars, S. D. *, P<0.05, **P<0.01. (FIG. 6C) lincRNA knockdown inhibits migration of MDA-MB-231 and MCF-7 cells in wound-healing assay. Cells were transiently transfected 30 hours before making scratches in the cell monolayer. Cell migration rate was analyzed with time-lapse microscopy. Lines—cells tracks analysed over 24 hours. Size bars—100 micrometers. (FIG. 6D) Quantification of MDA-MB-231 and MCF-7 cells migration distance over 24-hour time period. Bars—value of mean migration distance, error bars—S. D. (n=20-25 analyzed cells), ***P<0.001.

FIG. 7. Workflow of lincRNA pan-cancer analysis. TCGA RNA-Seq aligned data (BAM files) and raw data (fastq/SRA files) from several validation datasets were used in this study. Validation datasets were aligned using Tophat2. LincRNAs were quantified using the FeatureCounts program and then normalized as FPKM values. Tissue specificity and subtype analysis was performed on the entire lincRNA transcriptome. Differential expression was analyzed with DESeq2 to obtain a list of pan-cancer lincRNA biomarkers, followed by diagnostic classification modelling and survival analysis.

FIGS. 8A-B. Principal component analysis of mRNA expression in 12 TCGA datasets. The first three principal components (PCs) were plotted using the log FPKM values of lincRNA expression in (FIG. 8A) normal adjacent tissue and (FIG. 8B) cancer samples. The variances associated with each of the first 10 principal components are plotted alongside each graph (Scree Plot).

FIG. 9. Comparison of lincRNA tissue specificity between TCGA data and Cabili et al. Each plot is composed of the group of lincRNAs specific to a certain tissue type (liver, kidney etc), as defined in Human Body Map Project by Cabili et al. This group of lincRNAs are reassigned to specific tissues by the JS score calculated from the TCGA data. The correlations between studies in all tissue categories are significant.

FIG. 10. JSₜ scores for each tissue type. Each plot shows the scores for tissue specificity calculated for cancer and normal samples for each individual tissue type.

FIG. 11. Differential expression in each cancer type in the 12 TCGA cancer datasets. The significance threshold is set to α=0.05 after Benjamini-Hochberg correction.

FIG. 12. Normalized log 2 FPKM expression of the panel of six lincRNAs in the 12 TCGA cancer datasets.

FIG. 13. Validation of known prognostic lincRNAs. Differential expression analysis of known prognostic lincRNAs markers (PCAT1, MALAT1 and HOTAIR).

FIG. 14. Log2 fold change of the six lincRNA panel in the supplementary microarray validation datasets.

FIG. 15. LincRNA expression in the breast cancer cell lines from CCLE and GSE58135 compared with primary tumor expression levels.

FIG. 16. Correlation heatmap of expression levels among the six lincRNAs.

FIG. 17. Blast homology among all transcripts of the six lincRNAs.

FIG. 18. ROC of the diagnostic classifier in the TCGA training dataset.

FIGS. 19A-D. The prognostic potential of the pan-cancer lincRNA panel. (FIGS. 19A-D) The performance of the lincRNA panel in predicting survival is plotted with Kaplan-Meier curves. There were significant differences in overall survival for 463 BRCA patients (FIG. 19A), 350 OV patients with Grade 3 tumors, the dominant grade of TCGA OV (FIG. 19B), as well as the relapse free survival for 193 LUAD patients (FIG. 19C) and 139 LUSC patients (FIG. 19D). The higher and lower risk groups are separated by high and low prognostic index (PI) categories. The PI score is based on the Cox-Regression model of the six lincRNA panel.

FIGS. 20A-B. Additional cell line experiments on MDA-231 cell line, using siRNA #2 (less efficient siRNA). (FIG. 20A) lincRNA knockdown by siRNA #2 inhibits migration of MDA-MB-231 in wound-healing assay. Cells were transiently transfected 30 hours before making scratches in the cell monolayer. Cell migration rate was analyzed with time-lapse microscopy. Lines—cells tracks analyzed over 24 hours. Size bars—100 micrometers. (FIG. 20B) Quantification of MDA-MB-231 migration distance over 24-hour time period. Bars—value of mean migration distance, error bars—S. D. (n=60 analyzed cells), ***P<0.0001.

FIGS. 21A-C. Additional cell line experiments on HCT116 colon cancer cell line. (FIG. 21A) XLOC_12_004121 and XLOC_12_004340 lincRNAs can be efficiently knocked down in HCT116 cell lines. Bars—RT-qPCR results of XLOC_12_004121 (left bar in each grouping) and XLOC_12_004340 (right bar in each grouping) expression, shown as mean expression (n=3) normalized to GUS. (FIG. 21B) Transient knockdown of XLOC_12_004121 and XLOC_12_004340 inhibits the growth rate of HCT-116 cells. Data points represent mean value (n=3), error bars, S. D. *, P<0.05. (FIG. 21C) Quantification of HCT116 cells migration distance over 24-hour time period. Bars—value of mean migration distance, error bars—S. D. (n=60 analyzed cells), *, P=0.036.

### DETAILED DESCRIPTION

Long non-coding RNAs (lncRNAs) are a mysterious and recently discovered class of RNA molecules. The advancement of technologies has recently enabled identification of tens of thousands of novel lncRNAs. Many of these lncRNAs come from regions in the human genome that do not encode protein-coding genes, and therefore, they are called long intergenic non-coding RNAs (lincRNAs). Interestingly, even though lincRNAs do not code for proteins and are therefore thought to be regulatory RNAs, they often show similar characteristics as messenger RNA. However, the functions of most lincRNAs are unknown.

Cancer is a disease characterized by genetic mutations as well as changes in global gene expression. There is a growing recognition in the biomedical field that lincRNAs are also associated with tumor initiation and progression. Compared to protein coding genes, lincRNA expression patterns are much more specific to particular tissues or particular developmental stages, and are therefore potentially better candidates for cancer biomarkers.

Using a powerful data mining approach to search through thousands of pan-cancer samples and multiple cohorts (cancers from as many as ten organs including breast, lung, head and neck, colon, kidney, endometrial cancers), a panel of six lincRNAs that are highly accurate (97% accuracy) for the diagnosis of many types of cancers has been discovered. These lincRNAs are consistently up-regulated or down-regulated in ten cancer types. Patient survival analysis also demonstrates that their expression patterns are associated with prognosis in lung, breast and ovarian cancers. Cell culture experiments on two selected lincRNAs confirmed that they have effects on the growth and migration of breast and colon cancer cell lines. In summary, a panel of robust and accurate lincRNAs has been discovered for use as potential pan-cancer diagnostic and prognostic biomarker. This lincRNA panel has the potential to become a screening test for various types of cancers.

### Methods

As used herein, the term long intergenic non-coding RNAs (lincRNAs) refers to non-protein coding transcripts that are longer than 200 nucleotides and are transcribed from non-coding DNA sequences between protein coding genes.

As discussed herein, six lincRNAs were identified, which may be used as pan-cancer diagnostic and prognostic tools (see, Example 1). Specifically, it was discovered that PCAN-1 (i.e., XLOC_002996), PCAN-2 (i.e., XLOC_12_004121), PCAN-3 (i.e., XLOC_12_004340), PCAN-5 (i.e., XLOC_12_009441) and PCAN-6 (i.e., XLOC_12_013931) were consistently upregulated in cancer cells. Additionally, it was discovered that PCAN-4 (i.e., XLOC_12_007509) was consistently downregulated in cancer cells. The expression patterns of these lincRNAs were also associated with prognosis. The genomic coordinate descriptions of these six lincRNAs are shown in Table 3. Additionally, it is noted that numerous isoforms and variants of these lincRNAs exist and may be used to practice a method described herein. Thus, reference to each lincRNA (e.g., PCAN-1, PCAN-2, PCAN-3, PCAN-4, PCAN-5 or PCAN-6) includes isoforms or variants thereof.

Accordingly, certain embodiments of the invention provide a method for identifying a cancer cell, comprising detecting increased expression of at least one lincRNA selected from the group consisting of PCAN-1, PCAN-2, PCAN-3, PCAN-5 and PCAN-6 and/or decreased expression of lincRNA PCAN-4 in a nucleic acid sample derived from the cell, wherein increased expression of at least one of PCAN-1, PCAN-2, PCAN-3, PCAN-5 and PCAN-6 and/or decreased expression of PCAN-4, as compared to expression from a control cell, indicates the cell is a cancer cell. In certain embodiments, the cell is obtained from a biological sample taken from a patient.

Certain embodiments of the invention provide a method for identifying a cancer cell, comprising: 1) deriving a nucleic acid sample from the cell; and 2) detecting increased expression of at least one lincRNA selected from the group consisting of PCAN-1, PCAN-2, PCAN-3, PCAN-5 and PCAN-6 and/or decreased expression of lincRNA PCAN-4 in the nucleic acid sample; wherein increased expression of at least one of PCAN-1, PCAN-2, PCAN-3, PCAN-5 and PCAN-6 and/or decreased expression of PCAN-4, as compared to expression from a control cell, indicates the cell is a cancer cell. In certain embodiments, the cell is obtained from a biological sample taken from a patient.

Certain embodiments of the invention provide a method for identifying a cancer cell, comprising: 1) deriving a nucleic acid sample from the cell; 2) detecting whether expression of at least one lincRNA selected from the group consisting of PCAN-1, PCAN-2, PCAN-3, PCAN-5 and PCAN-6 is increased and/or whether expression of lincRNA PCAN-4 is decreased in the nucleic acid sample; and 3) identifying the cell as a cancer cell when increased expression of at least one of PCAN-1, PCAN-2, PCAN-3, PCAN-5 and PCAN-6 is detected and/or decreased expression of lincRNA PCAN-4 is detected, as compared to expression from a control cell. In certain embodiments, the cell is obtained from a biological sample taken from a patient.

Certain embodiments of the invention provide a method for detecting the presence of a biomarker in a cell, the improvement comprising detecting increased expression of at least one lincRNA selected from the group consisting of PCAN-1, PCAN-2, PCAN-3, PCAN-5 and PCAN-6 and/or decreased expression of lincRNA PCAN-4 in the cell for use in identifying the cell as a cancer cell, wherein increased expression of at least one lincRNA selected from the group consisting of PCAN-1, PCAN-2, PCAN-3, PCAN-5 and PCAN-6 and/or decreased expression of lincRNA PCAN-4 in the cancer cell, as compared to expression from a control cell, indicates the cell is a cancer cell.

Certain embodiments of the invention provide a method for identifying a patient having cancer, comprising detecting increased expression of at least one lincRNA selected from the group consisting of PCAN-1, PCAN-2, PCAN-3, PCAN-5 and PCAN-6 and/or decreased expression of lincRNA PCAN-4 in a nucleic acid sample that was derived from a biological sample obtained from the patient, wherein increased expression of at least one of PCAN-1, PCAN-2, PCAN-3, PCAN-5 and PCAN-6 and/or decreased expression of PCAN-4, as compared to expression from a control sample, indicates the patient has cancer.

Certain embodiments of the invention provide a method for identifying a patient having cancer comprising: 1) providing a nucleic acid sample that was derived from a biological sample obtained from the patient; and 2) detecting increased expression of at least one lincRNA selected from the group consisting of PCAN-1, PCAN-2, PCAN-3, PCAN-5 and PCAN-6 and/or decreased expression of lincRNA PCAN-4 in the nucleic acid sample; wherein increased expression of at least one of PCAN-1, PCAN-2, PCAN-3, PCAN-5 and PCAN-6 and/or decreased expression of PCAN-4, as compared to expression from a control sample, indicates the patient has cancer.

Certain embodiments of the invention provide a method for identifying a patient having cancer, comprising: 1) deriving a nucleic acid sample from a biological sample that was obtained from the patient; 2) detecting whether expression of at least one lincRNA selected from the group consisting of PCAN-1, PCAN-2, PCAN-3, PCAN-5 and PCAN-6 is increased and/or whether expression of lincRNA PCAN-4 is decreased by measuring the expression levels of PCAN-1, PCAN-2, PCAN-3, PCAN-4, PCAN-5 and/or PCAN-6; and 3) identifying the patient as having cancer when increased expression of at least one of PCAN-1, PCAN-2, PCAN-3, PCAN-5 and PCAN-6 is detected and/or decreased expression of lincRNA PCAN-4 is detected, as compared to expression from a control sample.

Certain embodiments of the invention provide a method for establishing a prognosis for a patient having cancer, comprising 1) detecting the expression levels of at least one lincRNA selected from the group consisting of PCAN-1, PCAN-2, PCAN-3, PCAN-4, PCAN-5 and PCAN-6 in a nucleic acid sample that was derived from biological sample obtained from the patient; and 2) comparing the expression levels to a relative cut-off value, wherein expression levels that are higher than the relative cut-off level for PCAN-1, PCAN-2, PCAN-3, PCAN-5 and/or PCAN-6 are indicative of a poor prognosis, and wherein expression levels that are lower than a relative cut-off level for PCAN-4 are indicative of a poor prognosis.

As used herein, the term “relative cut-off value” may be used to refer to a baseline, threshold, or percentile, such as the 25^{th}, 50^{th}, or 75^{th} percentile. For example, the prognosis for a patient may be poor when the expression level of PCAN-1 in a nucleic acid sample derived from the patient is higher than, e.g., the 50^{th} percentile, for PCAN-1 expression levels in cancer patients.

Certain embodiments of the invention provide a method for establishing a prognosis for a patient having cancer, comprising: 1) providing a nucleic acid sample that was derived from a biological sample obtained from the patient; 2) detecting the expression levels of at least one lincRNA selected from the group consisting of PCAN-1, PCAN-2, PCAN-3, PCAN-4, PCAN-5 and PCAN-6 in the nucleic acid sample; and 3) comparing the expression levels to a relative cut-off value, wherein expression levels of PCAN-1, PCAN-2, PCAN-3, PCAN-5 and/or PCAN-6 that are higher than a relative cut-off level are indicative of a poor prognosis and PCAN-4 expression levels that are lower than a relative cut-off level are indicative of a poor prognosis.

Certain embodiments of the invention provide a method for establishing a prognosis for a patient having cancer, comprising: 1) deriving a nucleic acid sample from a biological sample obtained from the patient; 2) detecting the expression levels of at least one lincRNA selected from the group consisting of PCAN-1, PCAN-2, PCAN-3, PCAN-4, PCAN-5 and PCAN-6 in the nucleic acid sample; 3) comparing the expression levels to a relative cut-off value; and 4) establishing the prognosis is poor when expression levels of PCAN-1, PCAN-2, PCAN-3, PCAN-5 and/or PCAN-6 are higher than the relative cut-off level and/or when PCAN-4 expression levels are lower than the relative cut-off level.

Certain embodiments of the invention provide a method for treating a cancer cell comprising contacting the cancer cell with an effective amount of a therapeutic agent, wherein the cancer cell was determined to comprise increased expression of at least one lincRNA selected from the group consisting of PCAN-1, PCAN-2, PCAN-3, PCAN-5 and PCAN-6 and/or decreased expression of lincRNA PCAN-4, as compared to expression from a control cell.

Certain embodiments of the invention provide a method for treating cancer in a patient comprising administering an effective amount of a therapeutic agent to the patient, wherein the cancer was determined to comprise increased expression of at least one lincRNA selected from the group consisting of PCAN-1, PCAN-2, PCAN-3, PCAN-5 and PCAN-6 and/or decreased expression of lincRNA PCAN-4, as compared to expression from a control.

Certain embodiments of the invention provide a therapeutic agent for the prophylactic or therapeutic treatment of a cancer determined to comprise increased expression of at least one lincRNA selected from the group consisting of PCAN-1, PCAN-2, PCAN-3, PCAN-5 and PCAN-6 and/or decreased expression of lincRNA PCAN-4, as compared to expression from a control.

Certain embodiments of the invention provide the use of a therapeutic agent to prepare a medicament for treating a cancer in a patient determined to comprise increased expression of at least one lincRNA selected from the group consisting of PCAN-1, PCAN-2, PCAN-3, PCAN-5 and PCAN-6 and/or decreased expression of lincRNA PCAN-4, as compared to expression from a control.

Certain embodiments of the invention provide a method comprising 1) detecting increased expression of at least one lincRNA selected from the group consisting of PCAN-1, PCAN-2, PCAN-3, PCAN-5 and PCAN-6 and/or decreased expression of lincRNA PCAN-4 in a nucleic acid sample that was derived from a biological sample obtained from a patient; 2) diagnosing the patient with cancer when increased expression of at least one lincRNA selected from the group consisting of PCAN-1, PCAN-2, PCAN-3, PCAN-5 and PCAN-6 is detected and/or decreased expression of lincRNA PCAN-4 is detected, as compared to expression from a control sample; and 3) administering an effective amount of a therapeutic agent to the patient.

Certain embodiments of the invention provide a method for identifying an effective cancer treatment in a patient, comprising:

1) detecting the expression level of at least one lincRNA selected from the group consisting of PCAN-1, PCAN-2, PCAN-3, PCAN-4, PCAN-5 and PCAN-6 in a first biological sample obtained from the patient before the cancer treatment;

2) detecting the expression level of at least one lincRNA selected from the group consisting of PCAN-1, PCAN-2, PCAN-3, PCAN-4, PCAN-5 and PCAN-6 in a second biological sample obtained from the patient after the cancer treatment; and

3) identifying the cancer treatment as effective based on the level of lincRNA expression, wherein decreased expression of at least one of PCAN-1, PCAN-2, PCAN-3, PCAN-5 or PCAN-6 in the second sample as compared to the first sample and/or increased expression of PCAN-4 in the second sample as compared to the first sample indicates that the cancer treatment is effective. In certain embodiments, the methods further comprise obtaining the first and the second biological samples from the patient.

Certain embodiments of the invention provide a method of screening a therapeutic agent for anti-cancer activity, comprising contacting a cancer cell comprising increased expression of at least one lincRNA selected from the group consisting of PCAN-1, PCAN-2, PCAN-3, PCAN-5 and PCAN-6 and/or decreased expression of lincRNA PCAN-4 (i.e., as compared to expression from a control) with a therapeutic agent, wherein sensitivity of the cancer cell to the therapeutic agent is indicative of anti-cancer activity.

As used herein, the term “sensitive to a therapeutic agent” and “sensitivity of a cancer cell to a therapeutic agent” refers to a cancer cell that has decreased growth, proliferation and/or dies when contacted with a therapeutic agent (e.g., a therapeutic agent is administered to a patient).

As used herein, the term “increased expression” refers to an increase in lincRNA expression levels. For example, the increase in expression may result from a mutation, gene amplification (i.e., an increase in gene copy number), increased transcription, or decreased degradation of the lincRNA. To establish whether expression is increased, expression levels may be compared to a control. For example, comparison may be made to the expression level of a corresponding lincRNA from a corresponding non-cancerous cell. Additionally, as described herein, expression may also be normalized using an internal control in certain embodiments.

As used herein, the term “decreased expression” refers to a decrease in lincRNA expression levels. For example, the decrease in expression may result from a genetic mutation (e.g., deletion), reduction in gene copy number, decreased transcription, or increased degradation of the lincRNA. To establish whether there is a loss/decrease of expression, expression levels may be compared to a control. For example, comparison may be made to the expression level of PCAN-4 from a corresponding non-cancerous cell. Additionally, as described herein, expression may also be normalized using an internal control in certain embodiments.

Accordingly, in certain embodiments, increased expression of at least one lincRNA selected from the group consisting of PCAN-1, PCAN-2, PCAN-3, PCAN-5 and PCAN-6 is detected/a cell comprises increased expression of PCAN-1, PCAN-2, PCAN-3, PCAN-5 and/or PCAN-6. In certain embodiments, expression levels are detected for more than one lincRNA (e.g., 1, 2, 3, 4, 5 or 6 or more). In certain embodiments, increased expression of more than one lincRNA is detected/a cell comprises increased expression of more than one lincRNA (e.g., 1, 2, 3, 4, 5 or 6 or more). For example, in certain embodiments increased expression of more than one lincRNA selected from PCAN-1, PCAN-2, PCAN-3, PCAN-5 and PCAN-6 is detected/a cell comprises increased expression of more than one of PCAN-1, PCAN-2, PCAN-3, PCAN-5 and PCAN-6. In certain embodiments, increased expression of PCAN-1 is detected. In certain embodiments, increased expression of PCAN-2 is detected. In certain embodiments, increased expression of PCAN-3 is detected. In certain embodiments, increased expression of PCAN-5 is detected. In certain embodiments, increased expression of PCAN-6 is detected. In certain embodiments, increased expression of PCAN-1, PCAN-2, PCAN-3, PCAN-5 and PCAN-6 is detected.

In certain embodiments, decreased expression of lincRNA PCAN-4 is detected.

In certain embodiments, increased expression of at least one lincRNA selected from the group consisting of PCAN-1, PCAN-2, PCAN-3, PCAN-5 and PCAN-6 is detected and decreased expression of lincRNA PCAN-4 is detected. In certain embodiments, increased expression of PCAN-1, PCAN-2, PCAN-3, PCAN-5 and PCAN-6 is detected and decreased expression of lincRNA PCAN-4 is detected.

In certain embodiments, expression of at least one of PCAN-1, PCAN-2, PCAN-3, PCAN-5 and PCAN-6 is increased by at least about 5%, 10%, 15%, 20%, 30%, 40%, 50%, 60%, 70%, 80%, 90%, 95% or more (e.g., as compared to expression of a corresponding lincRNA in a corresponding control cell, such as a corresponding non-cancerous cell).

In certain embodiments, expression of PCAN-4 is decreased by at least about 5%, 10%, 15%, 20%, 30%, 40%, 50%, 60%, 70%, 80%, 90%, 95% or more (e.g., as compared to expression of PCAN-4 in a corresponding control cell, such as a corresponding non-cancerous cell).

In certain embodiments, the cancer is a solid tumor cancer or the cancer cell is derived from a solid tumor cancer. In certain embodiments, the cancer or the cancer cell is a breast, head and neck, thyroid, colon, kidney, liver, lung, prostate, gastric, ovarian or endometrial cancer/cancer cell. In certain embodiments, the cancer or the cancer cell is breast cancer or a breast cancer cell. In certain embodiments, the cancer or the cancer cell is lung cancer or a lung cancer cell.

In certain embodiments, a method of the invention further comprises obtaining a biological sample from a patient for detecting the expression levels of a lincRNA selected from the group consisting of PCAN-1, PCAN-2, PCAN-3, PCAN-4, PCAN-5 and PCAN-6. In certain embodiments, the biological sample is a tissue sample. In certain embodiments, the biological sample is a blood sample (e.g., a plasma sample). In certain embodiments, the biological sample comprises cancer cells. In certain embodiments, a nucleic acid sample (e.g., DNA or RNA sample) is derived from the biological sample.

In certain embodiments, a method of the invention further comprises detecting increased expression of at least one lincRNA selected from the group consisting of PCAN-1, PCAN-2, PCAN-3, PCAN-5 and PCAN-6. In certain embodiments, a method of the invention further comprises detecting decreased expression of lincRNA PCAN-4. In certain embodiments, the lincRNA expression is detected using a method described herein.

In certain embodiments, a method of the invention further comprises generating a cDNA sample.

In certain embodiments, a method of the invention further comprises informing a patient for whom the increased expression of at least one lincRNA selected from the group consisting of PCAN-1, PCAN-2, PCAN-3, PCAN-5 and PCAN-6 is detected and/or decreased expression of lincRNA PCAN-4 is detected that they have cancer.

Methods for Detecting lincRNA Expression

A biological sample, according to any of the above methods, may be obtained using certain methods known to those skilled in the art. Biological samples may be obtained from vertebrate animals, and in particular, mammals. Tissue biopsy is often used to obtain a representative piece of tumor tissue. Alternatively, tumor cells can be obtained indirectly in the form of tissues or fluids that are known or thought to contain the tumor cells of interest. Variations in expression (lincRNA) may be detected from a tumor sample or from other body samples such as urine, sputum or blood (e.g., plasma, serum, etc.). Cancer cells are sloughed off from tumors and appear in such body samples. By screening such body samples, a simple early diagnosis can be achieved for diseases such as cancer. In addition, the progress of therapy can be monitored more easily by testing such body samples for variations in expression. Additionally, methods for enriching a tissue preparation for tumor cells are known in the art. For example, the tissue may be isolated from paraffin or cryostat sections (e.g., formalin-fixed paraffin-embedded (FFPE) tissue). Cancer cells may also be separated from normal cells by flow cytometry or laser capture microdissection.

A nucleic acid, may be e.g., genomic DNA, RNA transcribed from genomic DNA, or cDNA generated from RNA. A nucleic acid may be derived from a vertebrate, e.g., a mammal. A nucleic acid is said to be “derived from” a particular source if it is obtained directly from that source or if it is a copy of a nucleic acid found in that source.

In certain embodiments, genomic DNA is isolated from a biological sample (i.e., comprising cancer cells) and analyzed in the detection assay. In certain embodiments, RNA is isolated from a biological sample (e.g., comprising cancer cells) and analyzed in the detection assay. In certain embodiments, the methods further comprise reverse transcribing RNA isolated from the biological sample to generate cDNA.

In certain embodiments, the lincRNA expression is detected using reverse transcriptase-polymerase chain reaction (RT-PCR) methods, quantitative real-time PCR (qPCR), microarray, RNA sequencing (RNA-Seq), next generation RNA sequencing (deep sequencing).

In certain embodiments, the lincRNA expression is detected using quantitative real-time PCR (qPCR). In certain embodiments, qPCR is performed using at least one primer selected from the group consisting of:

(SEQ ID NO: 7)
Forward strand primer: 5′-AGCTTCGGAGAAGCAGTGGT-3′;
(SEQ ID NO: 8)
Reverse strand primer: 5′-TTCTTTCCGCGGAGACCT-3′;
(SEQ ID NO: 9)
Forward strand primer: 5′-ACAGATGAACCGCGGAGAC-3′;
(SEQ ID NO: 7)
Reverse strand primer: 5′-AGCTTCGGAGAAGCAGTGGT-3′;
(SEQ ID NO: 10)
Forward strand primer: 5′-TAAGGGTCATGGAGCTGGAG-3′;
(SEQ ID NO: 11)
Reverse strand primer: 5′-ATCAGCTCCTCCCCGAGTAT-3′;
(SEQ ID NO: 12)
Forward strand primer: 5′-GAAGTTTAATGTTGCCAATGGA-
3′;
(SEQ ID NO: 13)
Reverse strand primer: 5′-GCCTTTGCACAGACTGACCT-3′;
(SEQ ID NO: 14)
Forward strand primer: 5′-ATCCAGAACTGCAGCCAGTC-3′;
and
(SEQ ID NO: 15)
Reverse strand primer: 5′-AGAAGTACATGGGGGTGTGG-3′.

In certain embodiments, the lincRNA expression is detected using RNA sequencing (RNA-Seq) (e.g., ribosomal depletion RNA-Seq).

In certain embodiments, normalization controls are used in the detection assay (e.g., RNA expression from a housekeeping gene, such as GAPDH, beta actin, ribosomal protein genes, RPLPO, or GUS). Accordingly, in certain embodiments, the expression level of PCAN-1, PCAN-2, PCAN-3, PCAN-4, PCAN-5 and/or PCAN-6 in the biological sample is normalized to the level of a control RNA in the biological sample.

In certain embodiments, expression levels may be compared to expression levels from a control cell/sample to establish whether expression is increased or decreased. For example, expression may be compared to expression of a corresponding lincRNA from a corresponding non-cancerous cell (e.g., expression of PCAN-1 from a breast cancer cell could be compared to the expression of PCAN-1 from non-cancerous breast cell).

In certain embodiments of the invention, detecting the expression levels of a lincRNA in a nucleic acid sample may comprise contacting the sample with at least one oligonucleotide to form a hybridized nucleic acid. In certain embodiments, the at least one oligonucleotide is immobilized on a solid surface. In certain embodiments of the invention, the methods further comprise contacting the sample with a first oligonucleotide to form a first hybridized nucleic acid and contacting the sample with a second oligonucleotide to form a second hybridized nucleic acid.

In certain embodiments, the methods further comprise amplifying the hybridized nucleic acids. In certain embodiments, amplification of the hybridized nucleic acid is carried out by, e.g., polymerase chain reaction. In certain embodiments, the methods further comprise contacting the amplified nucleic acid(s) with a detection oligonucleotide probe, wherein the detection oligonucleotide probe hybridizes to the amplified nucleic acid(s).

According to the methods of the present invention, the amplification of nucleic acids present in a biological sample may be carried out by any means known to the art. Examples of suitable amplification techniques include, but are not limited to, polymerase chain reaction (including, for RNA amplification, reverse-transcriptase polymerase chain reaction), ligase chain reaction, strand displacement amplification, transcription-based amplification, self-sustained sequence replication (or “3SR”), the QP3 replicase system, nucleic acid sequence-based amplification (or “NASBA”), the repair chain reaction (or “RCR”), and boomerang DNA amplification (or “BDA”).

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable nucleic acid polymerase) with one oligonucleotide primer for each strand of the specific sequence to be detected under hybridizing conditions so that an extension product of each primer is synthesized that is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe described herein), the probe carrying a detectable label, and then detecting the label in accordance with known techniques. Where the nucleic acid to be amplified is RNA, amplification may be carried out by initial conversion to DNA by reverse transcriptase in accordance with known techniques.

### Therapeutic Agents

As described herein, the therapeutic agent may be any agent useful for treating cancer (e.g., a chemotherapeutic agent, hormonal agents or radiation therapy). Thus, in certain embodiments, the therapeutic agent is an anti-cancer agent. For example, anti-cancer agents include, but are not limited to, selective estrogen receptor modulators (SERMs) (e.g., tamoxifen, toremifene and fulvestrant), aromatase inhibitors (anastrozole, exemestane and letrozole), kinase inhibitors (imatinib mesulate, dasatinib, nilotinib, lapatinib, gefitinib, erlotinib, temsirolimus and everolimus), growth factor receptor inhibitors (e.g., Trastuzumab, cetuximab and panitumumab), regulators of gene expression (vorinostat, romidepsin, bexarotene, alitretinoin and tretinoin), apoptosis inducers (bortezomib and pralatrezate), angiogenesis inhibitors (bevacizumab, sorafenib, sunitinib and pazopanib), antibodies that trigger a specific-immune response by binding a cell-surface protein on lymphocytes (rituximab, alemtuzumab and ofatumumab), antibodies or other molecules that deliver toxic molecules specifically to cancer cells (tositumomab, ibritumomab tiuxetan, denileukin diftitox), cancer vaccines and gene therapy.

In certain embodiments, the therapeutic agent is a chemotherapeutic agent. Examples of chemotherapeutic agents that may be used in accordance with the methods described herein include, but are not limited to, 13-cis-Retinoic Acid, 2-Chlorodeoxyadenosine, 5-Azacitidine, 5-Fluorouracil, 6-Mercaptopurine, 6-Thioguanine, actinomycin-D, adriamycin, aldesleukin, alemtuzumab, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, azacytidine, bacillus calmette-guerin (BCG), bendamustine, bevacizumab, bexarotene, bicalutamide, bortezomib, bleomycin, busulfan, calcium leucovorin, citrovorum factor, capecitabine, canertinib, carboplatin, carmustine, cetuximab, chlorambucil, cisplatin, cladribine, cortisone, cyclophosphamide, cytarabine, darbepoetin alfa, dasatinib, daunomycin, decitabine, denileukin diftitox, dexamethasone, dexasone, dexrazoxane, dactinomycin, daunorubicin, decarbazine, docetaxel, doxorubicin, doxifluridine, eniluracil, epirubicin, epoetin alfa, erlotinib, eribulin, everolimus, exemestane, estramustine, etoposide, filgrastim, fluoxymesterone, fulvestrant, flavopiridol, floxuridine, fludarabine, fluorouracil, flutamide, gefitinib, gemcitabine, gemtuzumab ozogamicin, goserelin, granulocyte-colony stimulating factor, granulocyte macrophage-colony stimulating factor, hexamethylmelamine, hydrocortisone hydroxyurea, ibritumomab, interferon alpha, interleukin-2, interleukin-4, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, nelarabine, nilutamide, octreotide, oprelvekin, oxaliplatin, paclitaxel, palbociclib, pamidronate, pemetrexed, panitumumab, PEG Interferon, pegaspargase, pegfilgrastim, PEG-L-asparaginase, pentostatin, pertuzumab, plicamycin, prednisolone, prednisone, procarbazine, raloxifene, rituximab, romiplostim, ralitrexed, sapacitabine, sargramostim, satraplatin, sorafenib, sunitinib, semustine, streptozocin, tamoxifen, tegafur, tegafur-uracil, temsirolimus, temozolamide, teniposide, thalidomide, thioguanine, thiotepa, topotecan, toremifene, tositumomab, trastuzumab, tretinoin, trimitrexate, alrubicin, vincristine, vinblastine, vindestine, vinorelbine, vorinostat, or zoledronic acid.

In certain embodiments, the therapeutic agent affects the function of at least one lincRNA selected from PCAN-1, PCAN-2, PCAN-3, PCAN-4, PCAN-5 and PCAN-6.

In certain embodiments, the therapeutic agent inhibits the expression of at least one lincRNA selected from the group consisting of PCAN-1, PCAN-2, PCAN-3, PCAN-5 and PCAN-6.

In certain embodiments, the therapeutic agent inhibits cell proliferation and/or cell migration.

In certain embodiments, the therapeutic agent is an antisense nucleic acid capable of decreasing the expression of at least one lincRNA. In certain embodiments, the antisense nucleic acid is selected from the group consisting of siRNA, shRNA, or miRNA. In certain embodiments, the antisense nucleic acid is a siRNA. In certain embodiments, the siRNA inhibits the expression of PCAN-2 and/or PCAN3. In certain embodiments, the siRNA targets a lincRNA nucleic acid sequence selected from:

(SEQ ID NO: 5)
5′-UUCCUUUAGACCCAUUCUCUU-3′
and
(SEQ ID NO: 6)
5′-GAACCCACCACUGCUUCUC-3′.

In certain embodiments, the siRNA comprises a sense strand and antisense strand, wherein the sense strand is selected from:

5′-UUCCUUUAGACCCAUUCUCUU-3′ (SEQ ID NO:5) and

5′-GAACCCACCACUGCUUCUC-3′ (SEQ ID NO:6); and wherein the anti-sense strand comprises a sequence that is at least, e.g., about 70%, 75%, 80%, 85%, 90%, 95%, 99% or 100% complementary to the sense strand.

In certain embodiments, the siRNA comprises a sense strand and an antisense strand, wherein the sense strand comprises a sequence that has at least, e.g., about 80%, 85%, 90%, 95%, 99% or 100% sequence identity to 5′-UUCCUUUAGACCCAUUCUCUU-3′ (SEQ ID NO:5), and wherein the antisense strand comprises a sequence that has at least, e.g., about 80%, 85%, 90%, 95%, 99% or 100% sequence identity to 5′-AAGAGAAUGGGUCUAAAGGAA-3′ (SEQ ID NO:16). In certain embodiments, the sense and antisense strands comprise a sequence that is about 15 to about 25 nucleotides in length, or about 19 to about 21 nucleotides in length.

In certain embodiments, the siRNA comprises a sense strand and an antisense strand, wherein the sense strand comprises a sequence that has at least, e.g., about 80%, 85%, 90%, 95%, 99% or 100% sequence identity to 5′-GAACCCACCACUGCUUCUC-3′ (SEQ ID NO:6), and wherein the antisense strand comprises a sequence that has at least, e.g., about 80%, 85%, 90%, 95%, 99% or 100% sequence identity to 5′-GAGAAGCAGUGGUGGGUUC-3′ (SEQ ID NO: 17). In certain embodiments, the sense and antisense strands comprise a sequence that is about 15 to about 25 nucleotides in length, or about 17 to about 21 nucleotides in length.

Certain embodiments of the invention provide a siRNA described herein, such as a siRNA described above, as well as compositions comprising such siRNA.

In certain embodiments, the therapeutic agent increases the function of lincRNA PCAN-4.

In certain embodiments, the therapeutic agent increases the expression of lincRNA PCAN-4. In certain embodiments, the therapeutic agent comprises lincRNA PCAN-4. In certain embodiments, the therapeutic agent comprises a vector comprising a nucleic acid encoding lincRNA PCAN-4.

### Administration

A therapeutic agent can be formulated as a pharmaceutical composition and administered to a mammalian host, such as a human patient in a variety of forms adapted to the chosen route of administration, i.e., orally or parenterally, by intravenous, intramuscular, topical or subcutaneous routes.

Therapeutic agents can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the agents may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active agent may be combined with one or more excipients and used in the form of ingestible tablets, buccal tablets, troches, capsules, elixirs, suspensions, syrups, wafers, and the like. Such compositions and preparations should contain at least 0.1% of active agent. The percentage of the compositions and preparations may, of course, be varied and may conveniently be between about 2 to about 60% of the weight of a given unit dosage form. The amount of active agent in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active agent, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active agent may be incorporated into sustained-release preparations and devices.

The active agent may also be administered intravenously or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present agents may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present agents can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the therapeutic agents to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of a therapeutic agent can be determined by comparing their in vitro activity, and in vivo activity in animal models. Methods for the extrapolation of effective dosages in mice, and other animals, to humans are known to the art; for example, see U.S. Pat. No. 4,938,949.

The amount of the therapeutic agent, or an active salt or derivative thereof, required for use in treatment will vary not only with the particular salt selected but also with the route of administration, the nature of the condition being treated and the age and condition of the patient and will be ultimately at the discretion of the attendant physician or clinician.

The agent is conveniently formulated in unit dosage form. In one embodiment, the invention provides a composition comprising a therapeutic agent formulated in such a unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

A combination of therapeutic agents can also be administered, for example, a combination of agents that are useful for treating cancer. Examples of such agents include lincRNA inhibitors, chemotherapeutic agents or radiation therapies. Accordingly, one embodiment the invention also provides for the use of a lincRNA inhibitor (e.g., a siRNA targeting PCAN-1, PCAN-2, PCAN-3, PCAN-5 and/or PCAN-6), at least one other therapeutic agent, and a pharmaceutically acceptable diluent or carrier.

### Kits

Certain embodiments of the present invention provide kits for practicing methods of the invention, e.g., identifying a cancer cell/identifying a patient that has cancer. These kits contain packaging material, at least one reagent for detecting expression of at least one lincRNA described herein in a biological sample from the subject, and instructions for its intended use.

Certain embodiments of the invention provide a kit for identifying a cancer cell comprising 1) at least one reagent for detecting increased expression of at least lincRNA selected from the group consisting of PCAN-1, PCAN-2, PCAN-3, PCAN-5 and PCAN-6 and/or decreased expression of lincRNA PCAN-4 in a cell; and 2) instructions for using the reagent, wherein increased expression of at least one of PCAN-1, PCAN-2, PCAN-3, PCAN-5 and PCAN-6 and/or decreased expression of PCAN-4, as compared to expression from a control cell, indicates the cell a cancer cell.

Certain embodiments of the invention provide a kit for identifying a patient having cancer, comprising 1) at least one reagent for detecting increased expression of at least lincRNA selected from the group consisting of PCAN-1, PCAN-2, PCAN-3, PCAN-5 and PCAN-6 and/or decreased expression of lincRNA PCAN-4 in a nucleic acid sample that was derived from a biological sample obtained from the patient; and 2) instructions for using the reagent, wherein increased expression of at least one of PCAN-1, PCAN-2, PCAN-3, PCAN-5 and PCAN-6 and/or decreased expression of PCAN-4, as compared to expression from a control, indicates the patient has cancer.

In certain embodiments, the reagent is an oligonucleotide, such as a primer or a probe (e.g., a fluorescent probe). In certain embodiments, the primer is labeled and/or comprises a non-natural modification, such as a non-natural nucleotide.

The invention also provides a kit comprising a lincRNA inhibitor, at least one other therapeutic agent, packaging material, and instructions for administering the lincRNA inhibitor, and the other therapeutic agent or agents to an animal to treat cancer.

### Certain Definitions

The following definitions are used, unless otherwise described.

The term “polynucleotide” or “nucleic acid,” as used interchangeably herein, refers to polymers of nucleotides of any length, and include DNA and RNA (e.g., lincRNA). The nucleotides can be deoxyribonucleotides, ribonucleotides, modified nucleotides or bases, and/or their analogs, or any substrate that can be incorporated into a polymer by DNA or RNA polymerase.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. Long non-coding RNAs (lincRNAs) that are located within intergenic regions are referred to long intergenic non-coding RNAs (lincRNAs). “cDNA” refers to a single- or a double-stranded DNA that is complementary to and derived from RNA.

“Oligonucleotide,” as used herein, refers to short, single stranded polynucleotides that are at least about seven nucleotides in length and less than about 250 nucleotides in length. Oligonucleotides may be synthetic. The terms “oligonucleotide” and “polynucleotide” are not mutually exclusive. The description above for polynucleotides is equally and fully applicable to oligonucleotides.

“Oligonucleotide probe” can refer to a nucleic acid segment, such as a primer, that may be useful to amplify a sequence in the nucleic acid of interest (e.g., DNA (e.g., DNA encoding lincRNA, such as PCAN-1, PCAN-2, PCAN-3, PCAN-4, PCAN-5 and/or PCAN-6), RNA (e.g., lincRNA, such as PCAN-1, PCAN-2, PCAN-3, PCAN-4, PCAN-5 and/or PCAN-6), or cDNA) and that is complementary to, and hybridizes specifically to, a particular sequence in the nucleic acid of interest.

The term “primer” refers to a single stranded polynucleotide that is capable of hybridizing to a nucleic acid and allowing the polymerization of a complementary nucleic acid, generally by providing a free 3′-OH group.

The term “nucleotide variation” refers to a change in a nucleotide sequence (e.g., an insertion, deletion, inversion, or substitution of one or more nucleotides, such as a single nucleotide polymorphism (SNP)) relative to a reference sequence (e.g., a wild type sequence).

The term also encompasses the corresponding change in the complement of the nucleotide sequence, unless otherwise indicated. A nucleotide variation may be a somatic mutation or a germline polymorphism.

The term “copy number” or “copy number variant” refers to the number of copies of a particular gene in the genotype of an individual.

As used herein, the term “specifically hybridizes” or “specifically detects” refers to the ability of a nucleic acid molecule to hybridize to at least approximately six consecutive nucleotides of a sample nucleic acid.

In the context of the present invention, an “isolated” or “purified” nucleic acid molecule is a molecule that, by human intervention, exists apart from its native environment. An isolated nucleic acid molecule may exist in a purified form or may exist in a non-native environment. For example, an “isolated” or “purified” nucleic acid molecule, or portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences and proteins or partial-length proteins encoded thereby are also encompassed by the present invention.

By “fragment” or “portion” of a sequence is meant a full length or less than full length of the nucleotide sequence encoding, or the amino acid sequence of a polypeptide or protein. As it relates to a nucleic acid molecule, sequence or segment of the invention when linked to other sequences for expression, “portion” or “fragment” means a sequence having, for example, at least 80 nucleotides, at least 150 nucleotides, or at least 400 nucleotides. If not employed for expressing, a “portion” or “fragment” means, for example, at least 9, 12, 15, or at least 20, consecutive nucleotides, e.g., probes and primers (oligonucleotides), corresponding to the nucleotide sequence of the nucleic acid molecules of the invention. Alternatively, fragments or portions of a nucleotide sequence that are useful as hybridization probes generally do not encode fragment proteins retaining biological activity. Thus, fragments or portions of a nucleotide sequence may range from at least about 6 nucleotides, about 9, about 12 nucleotides, about 20 nucleotides, about 50 nucleotides, about 100 nucleotides or more.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have in at least one embodiment 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, 98%, to 99% sequence identity to the native (endogenous) nucleotide sequence.

“Synthetic” polynucleotides are those prepared by chemical synthesis.

“Recombinant nucleic acid molecule” is a combination of nucleic acid sequences that are joined together using recombinant DNA technology and procedures used to join together DNA sequences as described, for example, in Sambrook and Russell (2001).

The term “gene” is used broadly to refer to any segment of nucleic acid associated with a biological function. Genes include coding sequences and/or the regulatory sequences required for their expression. For example, gene refers to a nucleic acid fragment that expresses mRNA, functional RNA, or a specific protein, including its regulatory sequences. Genes also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. In addition, a “gene” or a “recombinant gene” refers to a nucleic acid molecule comprising an open reading frame and including at least one exon and (optionally) an intron sequence. The term “intron” refers to a DNA sequence present in a given gene which is not translated into protein and is generally found between exons.

A “vector” is defined to include, inter alia, any plasmid, cosmid, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation in a cell of an endogenous gene, transgene, as well as the transcription and stable accumulation of sense (mRNA) or functional RNA or other RNA, such as lincRNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule.

The terms “trans-acting sequence” and “trans-acting element” refer to DNA or RNA sequences whose function does not require them to be on the same molecule.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.

(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Certain methods of alignment of sequences for comparison are well known in the art. Thus, the determination of percent identity between any two sequences can be accomplished using a mathematical algorithm. Non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, J M B, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tₘ) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267 (1984); Tₘ 81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology Hybridization with Nucleic Acid Probes, part I chapter 2 “Overview of principles of hybridization and the strategy of nucleic acid probe assays” Elsevier, New York (1993). Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. The genes and nucleotide sequences of the invention include both the naturally occurring sequences as well as mutant forms.

“Naturally occurring,” “native” or “wild type” is used to describe an object that can be found in nature as distinct from being artificially produced. For example, a nucleotide sequence present in an organism (including a virus), which can be isolated from a source in nature and which has not been intentionally modified in the laboratory, is naturally occurring. Furthermore, “wild-type” refers to the normal gene, or organism found in nature without any known mutation.

“Somatic mutations” are those that occur only in certain tissues, e.g., in liver tissue, and are not inherited in the germline. “Germline” mutations can be found in any of a body's tissues and are inherited.

As used herein, the term “control”, “control cell”, “control sample” refers to a non-cancerous cell (e.g., a wildtype cell) or a sample from a subject that does not have cancer.

As used herein, the phrase “control RNA” can refer to a RNA whose expression remains constant and is not affected by cancer. In certain embodiments, the control RNA is encoded by a housekeeping gene, for example, GAPDH, beta actin, ribosomal protein genes, RPLPO, or GUS. In an alternative embodiment, the control RNA could be a lincRNA from a control sample.

The term “biomarker” is generally defined herein as a biological indicator, such as a particular molecular feature, that may affect or be related to diagnosing or predicting an individual's health.

The term “detection” includes any means of detecting, including direct and indirect detection.

The term “diagnosis” is used herein to refer to the identification or classification of a molecular or pathological state, disease or condition. For example, “diagnosis” may refer to identification of a particular type of cancer, e.g., breast cancer. “Diagnosis” may also refer to the classification of a particular type of cancer, e.g., by histology (e.g., a non small cell lung carcinoma), by molecular features (e.g., a lung cancer characterized by nucleotide and/or amino acid variation(s) in a particular gene or protein), or both.

The term “prognosis” is used herein to refer to the prediction of the likelihood of cancer-attributable death or progression, including, for example, recurrence, metastatic spread, and drug resistance, of a neoplastic disease, such as cancer.

The term “prediction” or (and variations such as predicting) is used herein to refer to the likelihood that a patient will respond either favorably or unfavorably to a drug or set of drugs. In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The terms “cancer” and “cancerous” refer to or describe the physiological condition in mammals that is typically characterized by unregulated cell growth and proliferation. Examples of cancer include, but are not limited to, carcinoma, lymphoma (e.g., Hodgkin's and non-Hodgkin's lymphoma), blastoma, sarcoma, and leukemia. More particular examples of cancers include squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (e.g., endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia and other lymphoproliferative disorders, and various types of head and neck cancer.

The term “treat”, “treatment” or “treating,” to the extent it relates to a disease or condition includes inhibiting the disease or condition, eliminating the disease or condition, and/or relieving one or more symptoms of the disease or condition.

The term “patient” as used herein refers to any animal including mammals such as humans, higher non-human primates, rodents domestic and farm animals such as cow, horses, dogs and cats. In one embodiment, the patient is a human patient.

The phrase “effective amount” means an amount of a compound described herein that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “long-term” survival is used herein to refer to survival for at least 1 year, 5 years, 8 years, or 10 years following therapeutic treatment.

The invention will now be illustrated by the following non-limiting Example.

### Example 1. Pan-Cancer Analyses Reveal lincRNAs Relevant to Tumor Diagnosis, Subtyping and Prognosis

### Abstract

Long intergenic noncoding RNAs (lincRNAs) are a relatively new class of non-coding RNAs that have the potential as cancer biomarkers. To seek a panel of lincRNAs as pan-cancer biomarkers, transcriptomes from over 3300 cancer samples with clinical information were analyzed. Compared to mRNA, lincRNAs exhibit significantly higher tissue specificities that are then diminished in cancer tissues. Moreover, lincRNA clustering results accurately classify tumor subtypes. Using RNA-Seq data from thousands of paired tumor and adjacent normal samples in The Cancer Genome Atlas (TCGA), six lincRNAs as potential pan-cancer diagnostic biomarkers (XLOC_002996, XLOC_12_004121, XLOC_12_004340, XLOC_12_007509, XLOC_12_009441 and XLOC_12_013931) were identified. These lincRNAs are robustly validated using cancer samples from four independent RNA-Seq data sets, and are verified by qPCR in both primary breast cancers and MCF-7 cell line. Interestingly, the expression levels of these six lincRNAs are also associated with prognosis in various cancers. The growth and migration dependence of breast and colon cancer cell lines on two of the identified lincRNAs were further experimentally explored. In summary, this study highlights the emerging role of lincRNAs as potentially powerful and biologically functional pan-cancer biomarkers and represents a significant leap forward in understanding the biological and clinical functions of lincRNAs in cancers. Of note:
LincRNAs exhibit significantly higher tissue specificities than mRNAs, which are then diminished in cancer tissues.
LincRNAs are highly deregulated in cancers and their expression strongly correlates with molecular subtypes
A panel of diagnostic lincRNA biomarkers were discovered using the pan-cancer samples of The Cancer Genome Atlas (TCGA), and further validated with multiple independent data sets.
Knocking down experiments of some pan-cancer up-regulated lincRNAs slow down the cell growth and migration in some cancer cell lines, suggesting that lincRNAs may be biologically functional.

### Research in Context

Most of the work on cancer characterization, diagnosis, prognosis and treatment have been focused on the protein coding genes. Long intergenic non-coding RNAs (lincRNAs) are a relatively new class of RNA molecules that are understudied for their biological and clinical functions. This report aims to expand our understanding on the roles of lincRNA. Specifically, the relevance of lincRNAs to tumor diagnosis, subtyping and prognosis was demonstrated. A panel of lincRNAs as pan-cancer diagnostic biomarkers is further proposed.

### Introduction

Advancement of high-throughput technologies such as RNA-Seq has recently allowed for the identification of tens of thousands of new lincRNAs in different tissues. The Encyclopedia of DNA Elements (ENCODE) project found that about 62% of the entire genome is transcribed to long (>200 base pairs) RNA sequences (Consortium, 2012). Given that 3% of the genome encodes protein-coding exons, the large majority of these transcripts are non-coding RNAs (lncRNAs). Among these lncRNAs, about one third come from intergenic regions (lincRNAs) (Consortium, 2012). Unlike small non-coding RNAs which may regulate target gene expression through simpler complementary recognition, the mechanisms of lincRNAs are complex and may depend on formation of RNA-protein complexes. Attempts have been made to extrapolate the functions of lincRNAs based on model lincRNAs, such as studies that predict lincRNAs binding to PRC2 or competing endogenous lincRNAs (micro-RNA “sponges”). However, lincRNAs remain one of the most mysterious and least understood species of non-coding RNAs.

Regardless of the regulatory mechanisms, lincRNAs are becoming a relatively new class of cancer biomarker candidates. The pan-cancer biomarker-based design of clinical trials, on the other hand, can increase statistical power and greatly decrease the size, expense, and duration of clinical trials (Cancer Genome Atlas Research et al., 2013). Towards this, a pan-cancer based lincRNA diagnostics biomarker study was proposed, which is aligned with the goal of The Cancer Genome Atlas (TCGA) analysis project that enables the discovery of novel adaptive, biomarker-based strategies to be practiced across boundaries of different tumor types (Cancer Genome Atlas Research et al., 2013).

In this study, the full advantage of the rich RNA-Seq data from the TCGA consortium was taken, as well as thousands of RNA-Seq and microarray data from Gene Expression Omnibus (GEO) and our own collection of breast cancer samples. By combining data-mining and machine-learning methods with biological function validation experiments, lincRNAs as a new paradigm for actionable diagnostics in the pan-cancer setting was highlighted. In addition, the comprehensive landscape of lincRNAs and their relationship to other omics data in pan-cancers was portrayed. It was found that the lincRNAs are more tissue-specific compared to protein-coding mRNAs, and they also convey complementary relevance to clinical information, including tumor molecular subtypes. Moreover, 6 lincRNAs were detected and thoroughly validated as potential pan-cancer diagnostic biomarkers in over 3300 tissue samples. Most of all, it was confirmed that the lincRNAs are biologically functional, by measuring the reduction of cell proliferation and migration in breast cancer cell lines with siRNA knockdown on two of the homologous lincRNAs.

### Materials and Methods

### RNA-Seq Datasets

### TCGA Datasets

12 cancer datasets from TCGA incorporating RNA-Seq data files from 1240 tissue samples (Table 1) were used. RNA-Seq datasets were chosen from cancers in TCGA that have at least 25 pairs of primary tumor and paired adjacent normal tissue samples. These datasets include breast invasive carcinoma (BRCA), colon adenocarcinoma (COAD), head and neck squamous cell carcinoma (HSNC), kidney chromophobe (KICH), kidney renal clear cell carcinoma (KIRC), kidney renal papillary cell carcinoma (KIRP), lung adenocarcinoma (LUAD), lung squamous cell carcinoma (LUSC), prostate adenocarcinoma (PRAD), stomach adenocarcinoma (STAD) and thyroid carcinoma (THCA). RNA-Seq BAM files were downloaded from UCSC Cancer Genomics Hub (https://cghub.ucsc.edu/) using the GeneTorrent program (Wilks et al., 2014). The TCGA alignment protocol used the Mapsplice alignment program (Wang et al., 2010) to align raw reads to the human genome, where loci with the same alignment score has equal probability to assign a read. Technical replicates were combined by merging the results from the BAM files. RefSeq genes and lincRNAs were quantified using featureCounts (Liao et al., 2013, Liao et al., 2014) from the Subread package (version 1.4.5-pl). RefSeq annotation was obtained from Illumina hg19 iGenomes and lincRNAs were obtained from Broad Institute Human Body Map project, so that a direct comparison of the tissue specificity results between TCGA samples and those in Cabili et al. (Cabili et al., 2011) could be made. All alignments were conducted on the New Hampshire INBRE (IDeA Network of Biomedical Research Excellence) grid computing system. Batch effect was corrected, and DESeq2 (Love et al., 2013, Love et al., 2014) (version 1.6.1) was used for calculating normalized count data and FPKM data. A combination of independent RNA-Seq and microarray datasets were used for verification, and the summary of the datasets is listed in Table 1.

### GEO Datasets

A large-scale search of GEO RNA-Seq database was performed to find additional datasets for verification. Datasets with tumor and normal samples with good read quality (read mapping rate and low duplication rates) were selected. These included GSE25599 (liver cancer), GSE58135 (breast cancer) and GSE50760 (colon cancer). In addition, normal breast tissue samples were taken from GSE52194, GSE45326 and GSE30611 for comparison with our cancer samples. GEO datasets were aligned to the UCSC hg19 genome using Tophat2 with default parameters for either single-end or paired-end protocols. LincRNA count quantification and FPKM data were generated as above. Microarray datasets from GEO with tumor and normal samples were selected based on platforms that had probes mapping to the six lincRNAs of interest.

### Our Own Dataset

Our primary breast cancer samples were extracted with RNeasy Mini Kit (Qiagen), followed by quality control with RNA 6000 chips (Agilent Bioanalyzer). RNA species with RIN values >7 were sent to the Genomics Core of Yale Stem Cell Centre. Ribo-depleted RNA-Seq was conducted with 100 bp read length. The read count quantification and FPKM data were generated as above. The RNA-Seq reads of our samples will be deposited to GEO upon publishing of this manuscript.

### Tissue Specificity

To analyze tissue specificity, Jensen-Shannon divergence score (JS score) was calculated from tumor and normal samples of each tissue, and the two distributions of JS scores were compared following the method of Cabili et al. (Cabili et al., 2011). Briefly, fragments per kilo bases of exons for per million mapped reads (FPKM) were first calculated from the normalized count data from each sample. Then the mean FPKM for each tissue type was calculated and log transformed. The vector e that represents the distribution of expression is given by:

$$e = \frac{{log}_{2} ( FPKM + 1 )}{{\sum }_{i = 1}^{n} {log}_{2} ( {FPKM}_{i} + 1 )}$$

The JSₜ score is the JS score for each tissue type t, calculated by the following:

$${JS}_{t} ( e , {e}^{t} ) = 1 - \sqrt{H ( e + {e}^{t} ) - \frac{H ( e ) + H ( {e}^{t} )}{2}}$$

Where H is the Shannon entropy and e^{t} is the hypothetical distribution when a lincRNA is expressed in only one tissue type:

$${e}^{t} = ( {e}^{1} , … , {e}^{i} , … , {e}^{n} ) , where {e}^{i} = \{ \begin{matrix}1 & if i = t \\ 0 & if i \neq t\end{matrix}$$

The JS score for a lincRNA is then defined as the maximum St score across all tissue types.

### Differential Expression

Each of the 12 TCGA cancer datasets was tested for differential expression (DE) using DESeq2 (Love et al., 2013, Love et al., 2014). Statistically significant genes were selected with a FDR adjusted p-value threshold of 0.05 after Benjamini & Hochberg multiple hypothesis correction. As a result, six lincRNAs were discovered to be consistently upregulated or down-regulated in all twelve TCGA cancer datasets. These six lincRNAs were used subsequently for survival and pathway analysis.

### Survival Analysis

These six lincRNAs with pan-cancer diagnostic potential were examined for their association with patient survival among four types of TCGA cancer types. Note that these lincRNAs were initially selected as diagnostic biomarkers, but not prognostic biomarkers. The survival data from the four types TCGA cancers were obtained in two approaches. LUAD, LUSC and OV have relapse free survival information directly available from the TCGA data repository. The fourth cancer type BRCA has overall survival data available, per the courtesy of Volinia et al (Volinia and Croce, 2013). Patients who did not have an event (death or tumor relapse, depending on the data set) during the study were considered as censored. The expression values of the six lincRNAs were used as predictors to fit a Cox-Proportional Hazards (Cox-PH) regression model, where the overall survival or disease free survival was the response variable. For each patient, a prognosis index (PI) score was generated from the Cox-PH model. The median PI score among all patients of the same cancer type was used as the threshold to dichotomize the patients into high vs. low risk groups, similar to others. The log-rank p-value was then calculated to assess the statistically significant difference between the Kaplan-Meier curves of the high vs. low risk groups.

### Tumor Subtype Classification and Concordance Between Data Types Using NMF

Non-negative matrix factorization (NMF) method was used to classify tumor subtypes with lincRNA expression values. The optimal number of clusters was selected using the maximum cophenetic correlation. The lincRNA clustering results were then compared to those of other data types, using the method similar to Han et al (Han et al., 2014). The other data types from the TCGA include mRNA-Seq, mature microRNA-Seq, methylation and reverse phase protein array (RPPA) for each cancer type, all obtained from the Broad institute Genomic Data Analysis Center (GDAC). The concordances from the chi-square tests between lincRNA and other data types were used to assess the correlations between clustering.

Additionally, lincRNA clustering was compared with another standard method, the PAM50 clustering (Cancer Genome Atlas, 2012), using the TCGA breast cancer samples. The correlation between these two clustering approaches was calculated using the concordance as mentioned above. Similarly, cluster correlation was computed for subtypes based on ER+/−information from the GSE58135 breast cancer dataset.

### LincRNA Sequence Coding Potential and Homology Characterization

To predict the coding potential of the sequences, iSeeRNA (Sun et al., 2013) and Coding-Potential Assessment Tool (CPAT) (Wang et al., 2013) were used. The two programs are trained on long non-coding RNAs to assess the coding potential of transcripts. For iSeeRNA, the coordinates of lincRNA transcripts and exons were used as inputs in the form of GFF files. For CPAT, lincRNA sequences were used as inputs in the form of fasta files. To test for homology between transcripts, NCBI's command line BLAST+ suite (Camacho et al., 2009) was used. Pairwise BLAST was performed on all isoforms of the six differentially expressed lincRNAs. The percentages of homology were calculated by the number of matching base pairs divided by the total number of base pairs in the query sequence. Due to the high homology between three of the discovered lincRNAs (XLOC_12_004121, XLOC_12_004340 and XLOC_12_009441), downloaded RNA-Seq reads may have slight ambiguity in counting these lincRNA expression, since they were generated by TCGA using the Mapsplice alignment program (Wang et al., 2010).

Quantitative RT-PCR (qRT-PCR) Analysis

Total RNA from MDA-MB-231 and MCF-7 cell lines was isolated using RNeasy Mini Kit (Qiagen). Pooled total RNA from five healthy normal breast cancer patients was ordered from Biochain (Total RNA—Human Adult Normal Tissue 5 Donor Pool: Breast, catalog# R1234086-P). To match these healthy controls, total RNA was isolated from five in-house breast cancer patient samples.

High Capacity cDNA Reverse Transcription kit (Life Technologies, Thermo Scientific) was used for random-primed first-strand complementary DNA synthesis. Real time quantitative PCR (qPCR) was performed with SYBR Green (Life Technologies) with primers against selected linc RNAs (primer sequences are listed in Table 6). Amplification and real time measurement of PCR products was performed with 7900HT Fast Real-Time PCR System (Life Technologies). The comparative Ct method (Livak and Schmittgen, 2001) was used to quantify the expression levels of lincRNAs. Beta-glucuronidase (GUS) gene expression served as the internal control. GUS was selected as the internal control, as its expression level has been found to be comparable in range to the expression of linc RNAs and is stable in a wide variety of cancers (Habel et al., 2006, Rubie et al., 2005).

### RNA Interference

The siRNA oligos were synthesized by GE Dharmacon. The target sequences are as follows: control siRNA: 5′-UGGUUUACAUGUCGACUAA-3′(SEQ ID NO:1), 5′-UGGUUUACAUGUUGUGUGA-3′(SEQ ID NO:2), 5′-UGGUUUACAUGUUUUCUGA-3′(SEQ ID NO:3), 5′-UGGUUUACAUGUUUUCCUA-3′(SEQ ID NO:4); lincRNA siRNA #1: 5′-UUCCUUUAGACCCAUUCUCUU-3′ (SEQ ID NO:5); lincRNA siRNA #2: 5′-GAACCCACCACUGCUUCUC-3′ (SEQ ID NO:6). This lincRNA siRNA targets XLOC_12_004121 and XLOC_12_004340 lincRNAs. Cells were transfected in a 6-well plate format with siRNA oligos at 40 nM (for cell proliferation assays) or 60 nM (for migration assays) concentration, using DharmaFECT 1 Transfection Reagent (Dharmacon). The knockdown efficiency was determined by qRT-PCR 24 hours post transfection.

### Cell Growth and Migration Assays

Cell proliferation analysis was done using CellTiter-Glo Luminescent Cell Viability Assay Kit (Promega). Briefly, MDA-MB-231 cells were transfected in biological triplicates with siRNA constructs (control siRNA and linc RNA siRNA). After 24 hours, 400 cells of each condition were seeded in triplicates into 96-well plates and allowed to grow for another 48 hours. Cells number estimation at different time points was based on the quantification of the present ATP using SpectraMax Gemini XPS microplate reader (Molecular Devices). Cell migration was analysed using well established wound-healing assay. Scratches in cell monolayer were made 30 hours post siRNA transfection (3 scratches in each of the 3 biological replicates). Cell migration was analysed by time-lapse microscopy using IX81 Olympus microscope, with 10× objective (for MDA-MB-231 cells) and 4× objective with additional 1.6× magnification (for MCF-7 cells). Images were taken every 5 minutes over time period of 24 hours. Migration rates and cell tracking were analysed using the Metamorph software.

### Results

### Overview of the Workflow

To detect genes differentially expressed between healthy and tumor tissues, a two-factor (cancer/normal, and source of samples) experimental design was employed in which patients with tumor samples and matched normal sample were selected. This approach allowed sufficient statistical power by reducing the variation of data (Ching et al., 2014). In total, 1240 paired cancer and adjacent normal RNA-Seq samples in 12 different cancer types were downloaded.

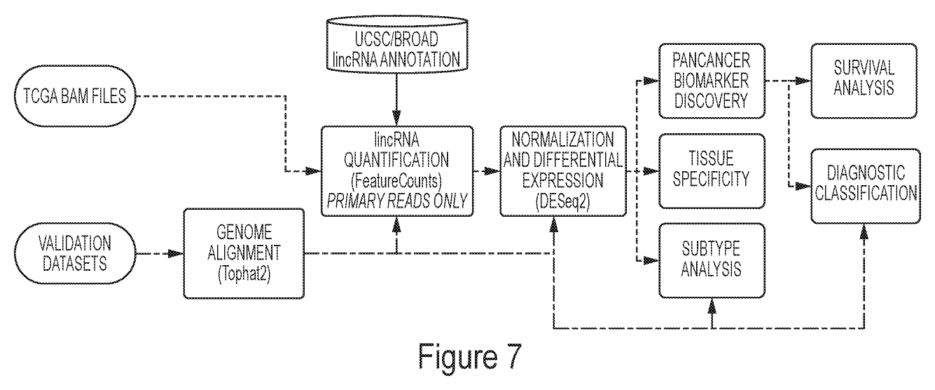

The 12 different cancer types include breast invasive carcinoma (BRCA), colon adenocarcinoma (COAD), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), prostate adenocarcinoma (PRAD), stomach adenocarcinoma (STAD) and thyroid carcinoma (THCA). Details on the number of samples in each cancer type, sequencing strategies, total mappable reads, and detected lincRNAs are listed in Table 1. For lincRNA genomic coordinates, the UCSC genome browser's “lincRNA transcript track” was used, which is based on both the Broad Institute Human Body Map including the annotations of transcripts of uncertain coding potential (TUCP) (Cabili et al., 2011). lincRNA expression was quantified with normalized fragments per kilobase per million (FPKM) values. Computationally, various analyses were performed to study the biological and clinical relevance of lincRNAs to pan-cancer, including differential expression (DE), tissue specificity and molecular subtype analyses, as well as construction and verification of the diagnostic and survival models (FIG. 7). Experimentally, the gene expression differences of a panel of 6 lincRNAs were verified, which have pan-cancer diagnostic biomarker potential. Importantly, the phenotypic changes were demonstrated for two of the over-expressed lincRNAs by siRNA knockdown experiments in two breast cancer cell lines MCF-7 and MDA-MB-231.

The High Tissue Specificities of lincRNAs are Diminished in Cancers

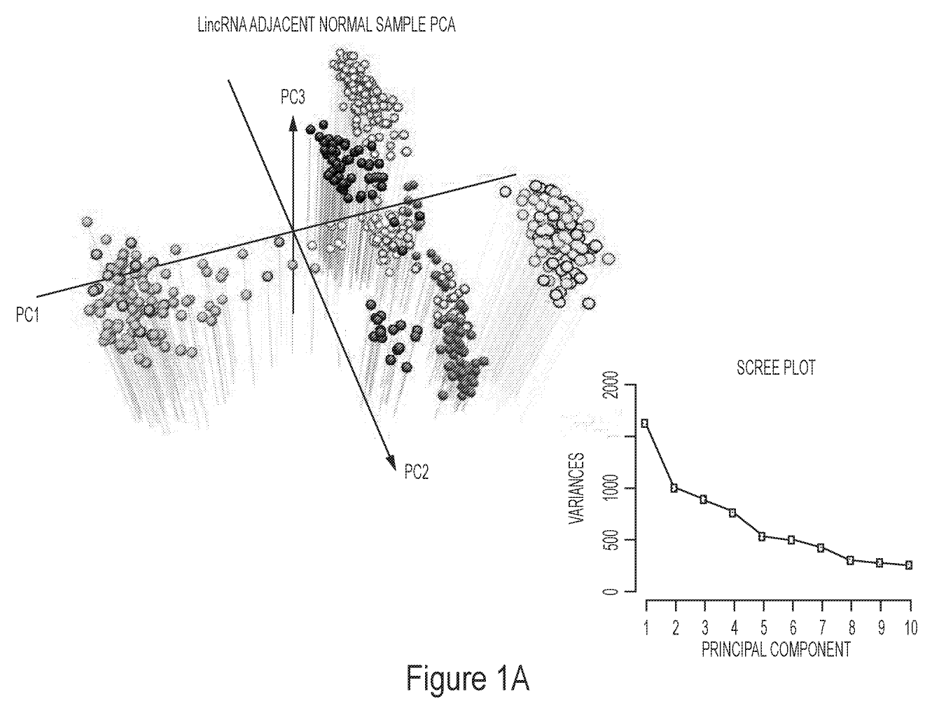

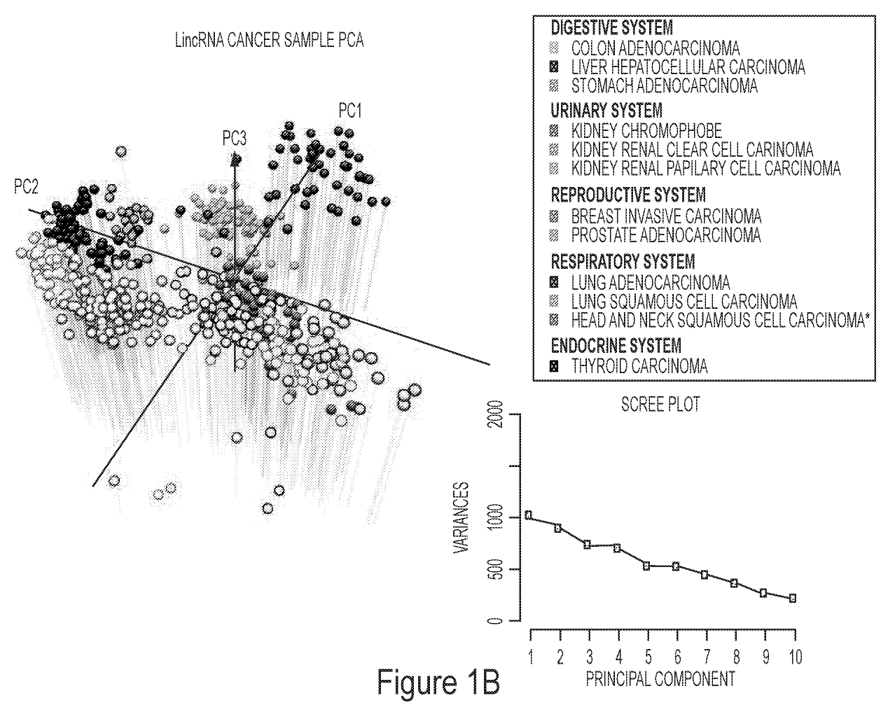

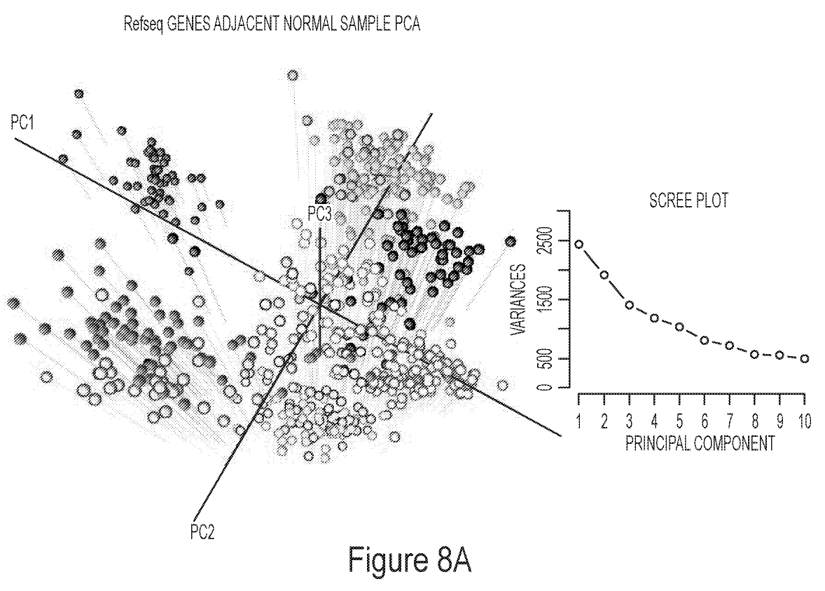

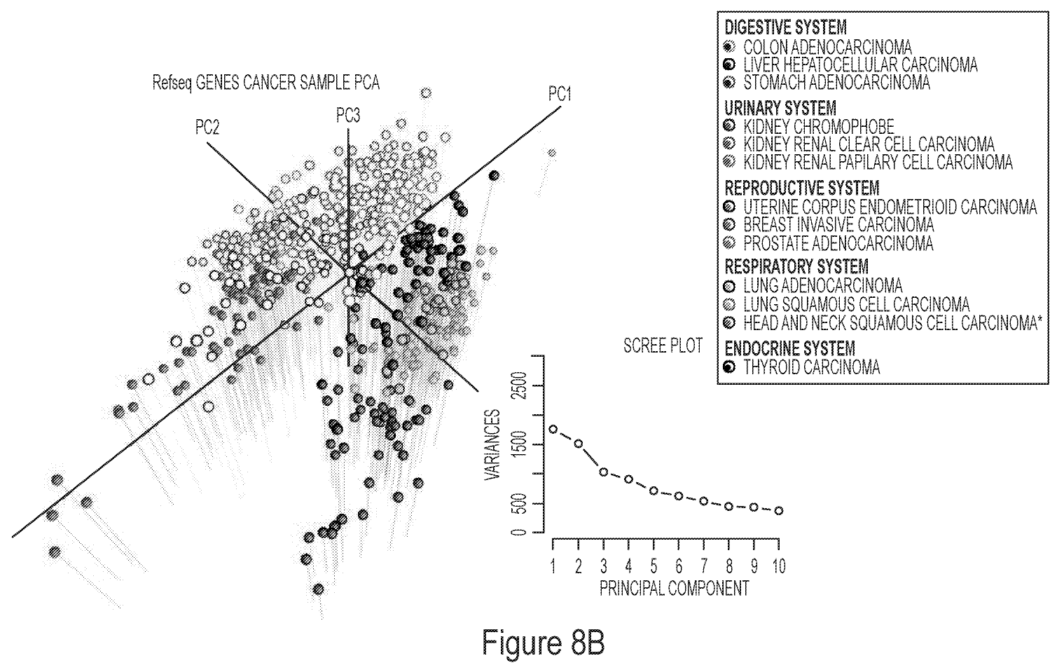

To investigate the expression patterns of the lincRNA transcripts among different tissue types, principal component analysis (PCA) was conducted for lincRNA expression on adjacent normal and cancer samples separately from 12 TCGA datasets (FIGS. 1A-B). As expected, the normal samples are clearly clustered by tissue type based on lincRNA expression (FIG. 1A). However, the cancer samples become less separable by tissue type (FIG. 1B). The less precise distinction of cancer samples in the PCA plot reflects a degree of de-differentiation of tumor cells. The possibility of confounding due to heterogeneity of tumors of the same type can be excluded, since the latter would lead to more spreading, rather than less spreading observed on the PCA plot. It was therefore reasoned it as the loss of tissue specificity in cancers. Supporting this observation, the first three principal components of PCA account for less variance in cancer samples compared to those in the adjacent normal tissues, suggesting deregulation of lincRNAs in cancers (FIGS. 1A-B). The same analysis was replicated for protein-coding genes between tumor and adjacent normal tissues, and found the same trend of losing tissue specificity in the tumor samples (FIGS. 8A-B).

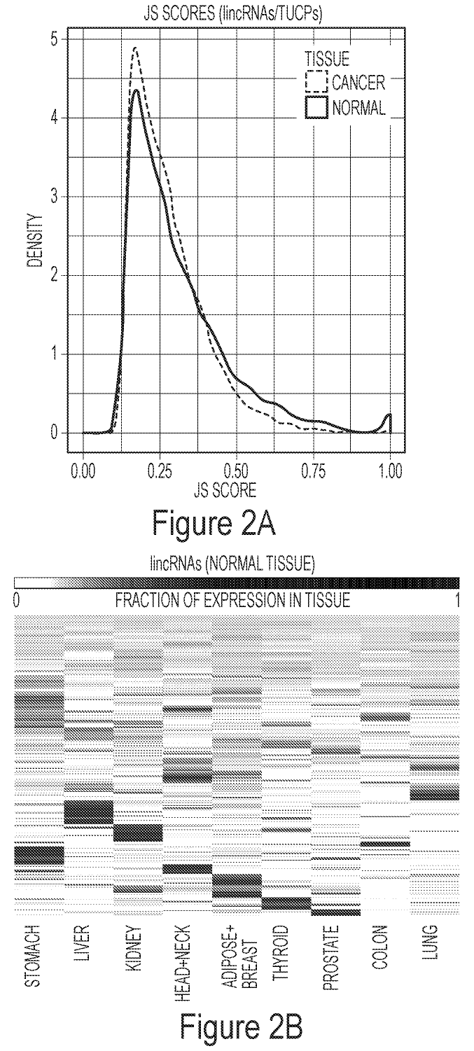

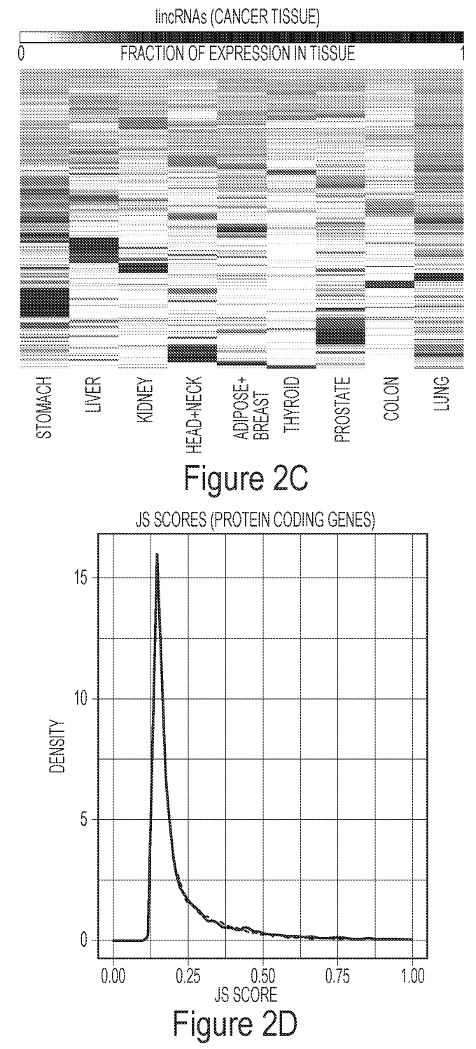

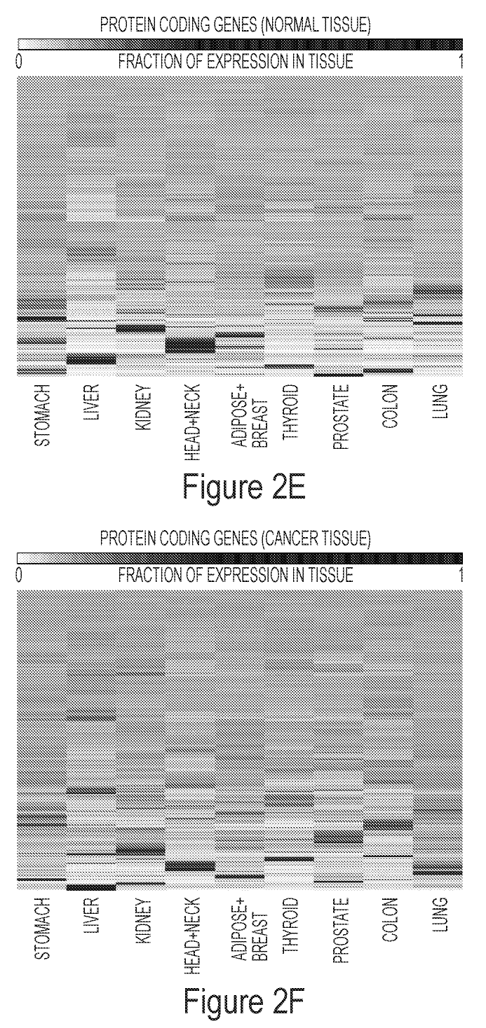

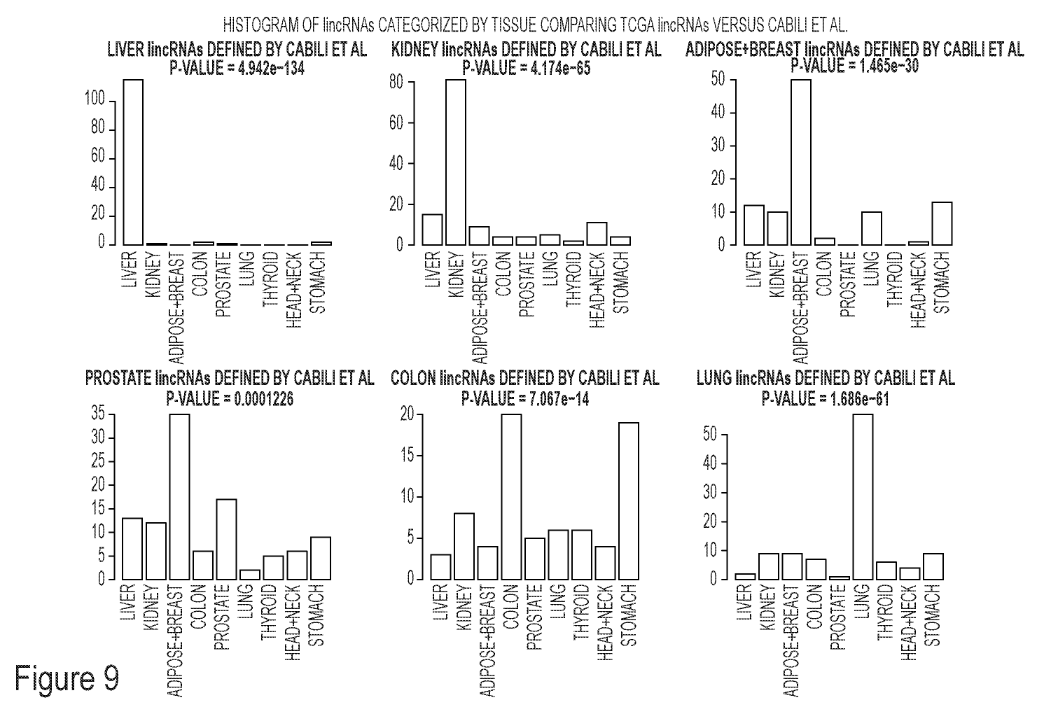

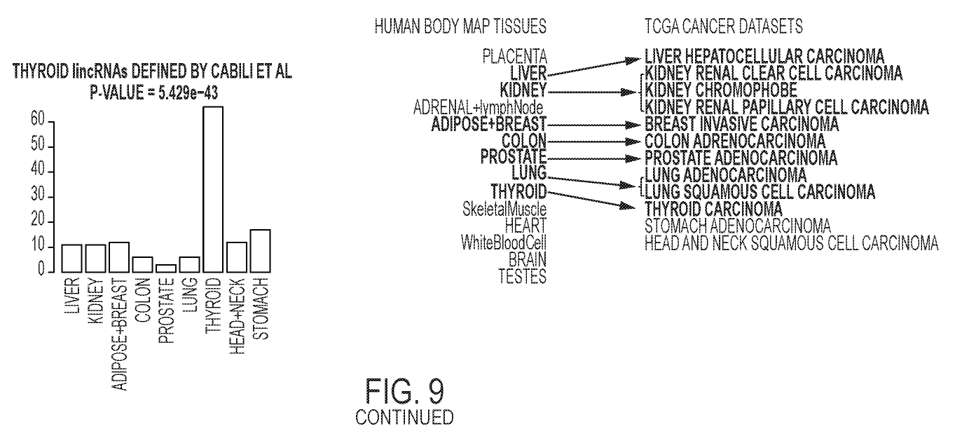

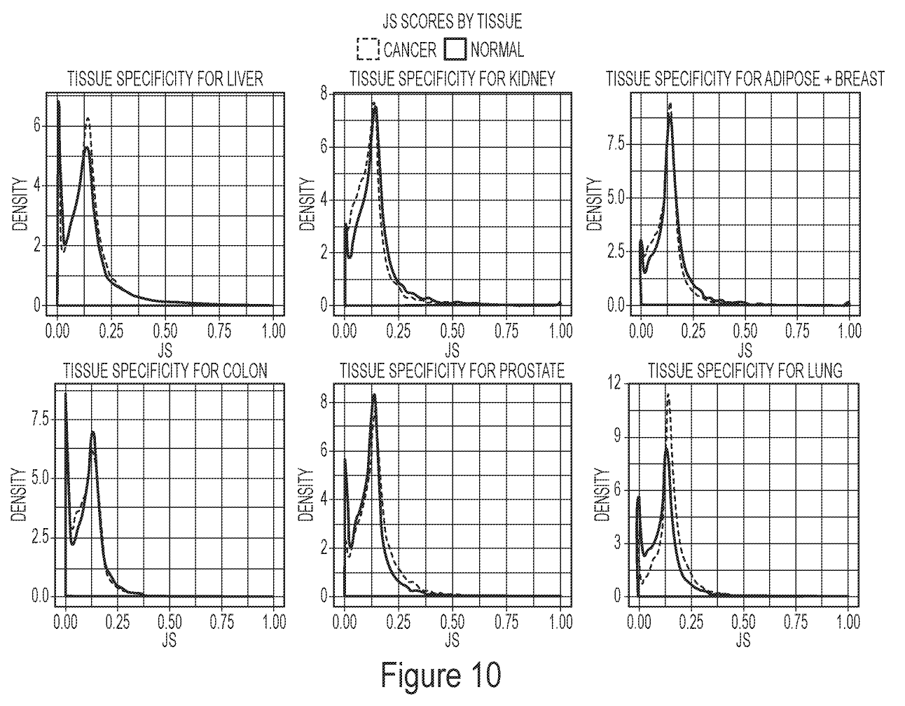

To further analyze the tissue specificity of lincRNAs, the tissue specificity scores (JS scores) as defined in Cabili et al. was calculated, where a higher JS score indicates more tissue specificity. The distributions of these JS scores in tumor and adjacent normal tissue were compared, for both lincRNAs and RefSeq protein coding genes (FIGS. 2A-F). Consistent with the PCA plots, lincRNAs in cancer tissues are significantly less tissue specific than those in adjacent normal tissues (t-test, p<2.2e-16) (FIGS. 2A, C and D). Moreover, in comparison with RefSeq protein coding genes (FIGS. 2B, E and F), lincRNAs have a much higher average JS score (t-test, p<2.2e-16). Subsequently, a subset of lincRNAs was defined that are highly tissue specific with JS score greater than 0.75 and are expressed in at least 5% (32 out of 640) of the total normal samples (Table 2). To confirm that the tissue-specific lincRNAs defined by TCGA pan-cancer analysis are accurate, the tissue type assigned to lincRNAs by Cabili et al. was then compared to the tissue types assigned to the same lincRNAs based on the TCGA data. Statistically significant correlations (χ²-test, all p<0.0001) were observed between the two studies in all tissue categories (FIG. 9). In addition, the tissue specific JS score was plotted for each tissue type (JSₜ score), as well as their distributions (FIG. 10). As expected, significant amounts of lincRNA have zero JS scores, as many lincRNAs are not expressed in certain tissues.

### LincRNA Clustering Accurately Predicts Molecular Subtypes of Tumors

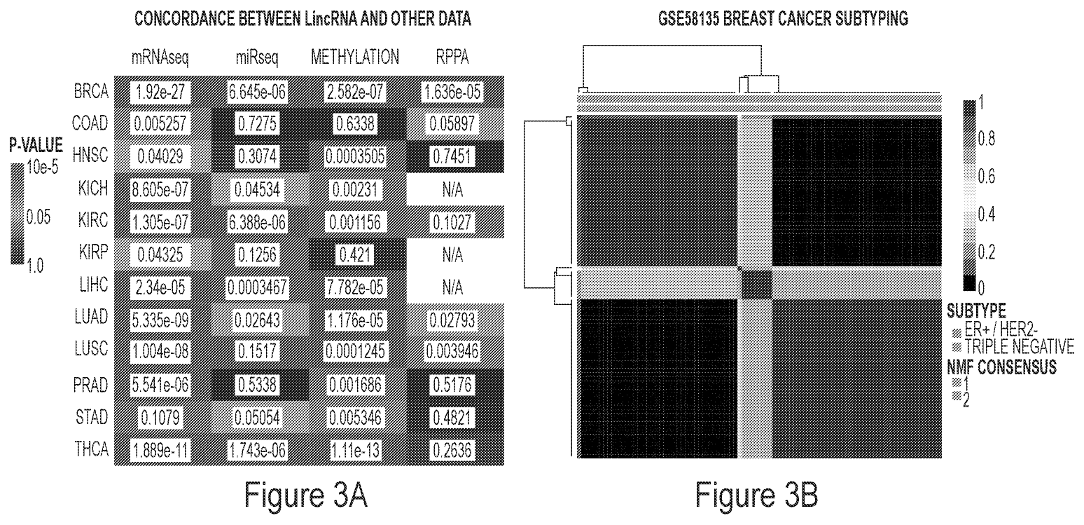

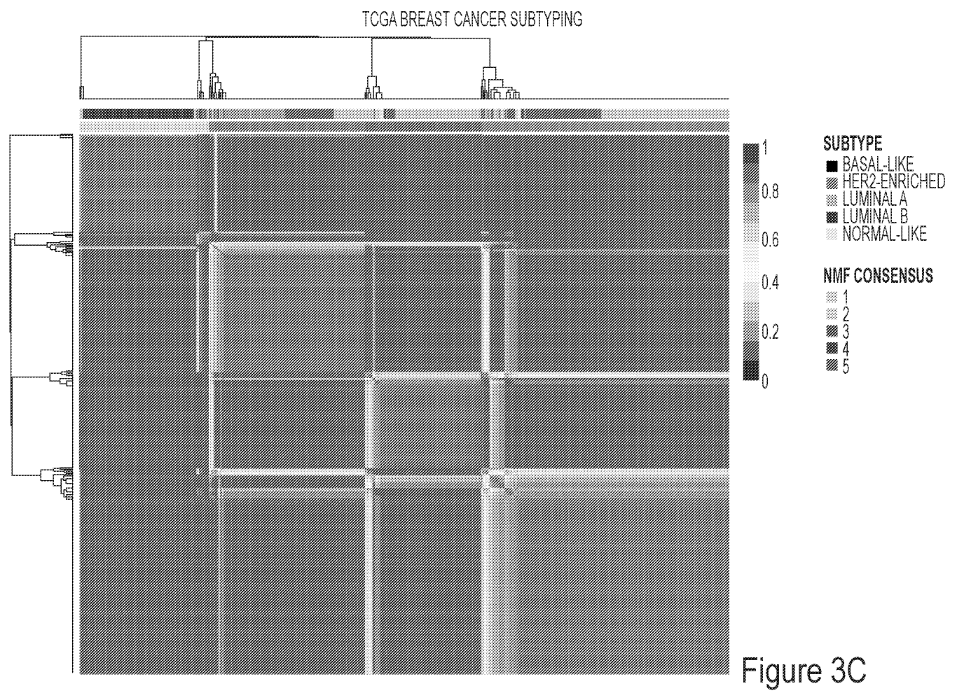

Given the tissue specificity of lincRNAs, it was hypothesized that lincRNAs can accurately separate tumors by molecular subtype. To identify a representative cancer type, consensus non-negative matrix factorization (CNMF) was first used to cluster the patient samples from each of the 12 types of cancer. The correlations between the clustering result was then calculated based on lincRNAs and those based on four other high-throughput data types: mRNA expression, micro-RNA expression, DNA methylation and reverse phase protein array (RPPA) obtained from the Broad Institute Genomic Data Analysis Center (GDAC) (Broad, 2014). The majority of lincRNA and GDAC clustering results are statistically significantly correlated (FIG. 3A). As expected, lincRNA and mRNA expression are the most highly correlated among all four high-throughput data types. Among the 12 cancer datasets, the BRCA dataset has the best agreements between lincRNAs and the other data types. Therefore, there was a focus on the correlation between lincRNA and molecular subtypes in breast invasive carcinoma.

CNMF was first applied to the TCGA BRCA dataset and used cophenetic correlation (Liao et al., 2013) to determine the optimal cluster number to be 5, the same number of clusters as in PAM50 based classification. The result of CNMF clustering was then compared to PAM50 based subtypes, which include basal-like, HER2-enriched, luminal A, luminal B and normal-like subtypes (Cancer Genome Atlas, 2012) (FIG. 3C). The concordance score based on the χ²-test is highly significant (p<2.2e-16), and the overall accuracy to clinical types is 71.6%, as measured by rand measure, a metric for the percentage of agreement on a pair of samples belonging to the same group. Interestingly, the first CNMF cluster has the strongest correlation with the basal-like subtype among all molecular subtypes, with an accuracy of 95% based on rand measure. Additionally, the GSE58135 breast cancer dataset was examined that has primary tumor samples in ER+/HER2- and triple negative subtypes (FIG. 3B). The unsupervised CNMF clustering on these cancer samples yields highly accurate separation between ER+/HER2- and triple negative samples (χ²-test p<2.2e-16, and rand measure 84.5%). These results show that lincRNAs are well correlated with the molecular subtypes of tumors.

Transcriptome Analysis Reveals a Pan-Cancer Panel of Six lincRNAs

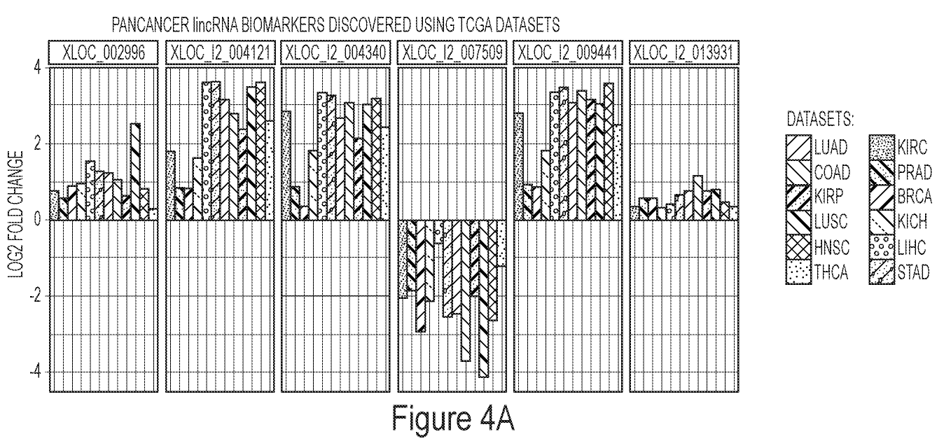

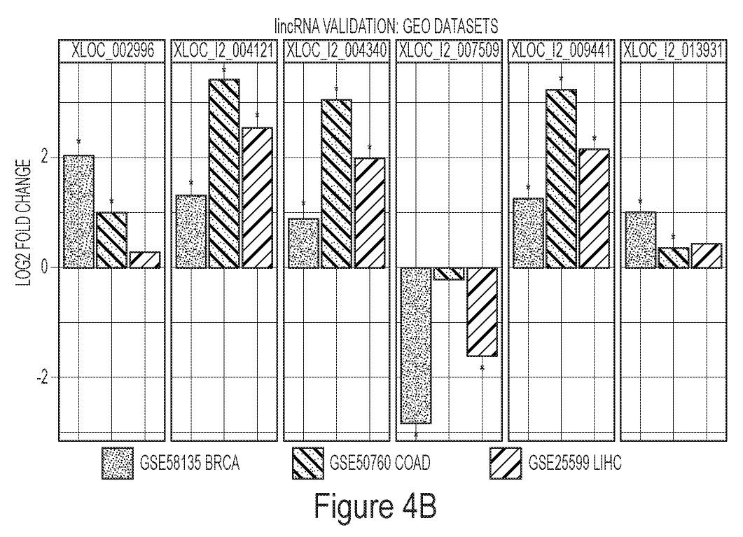

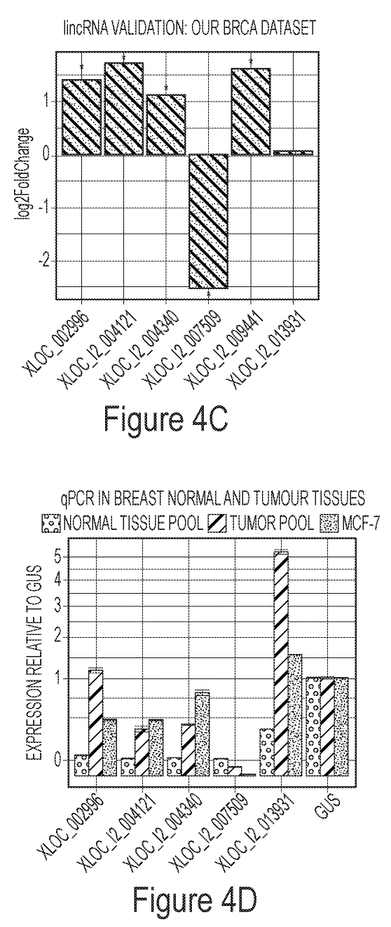

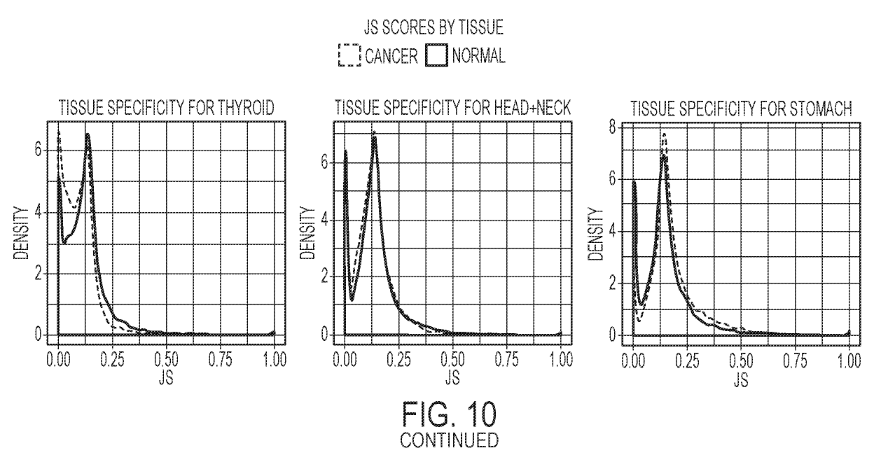

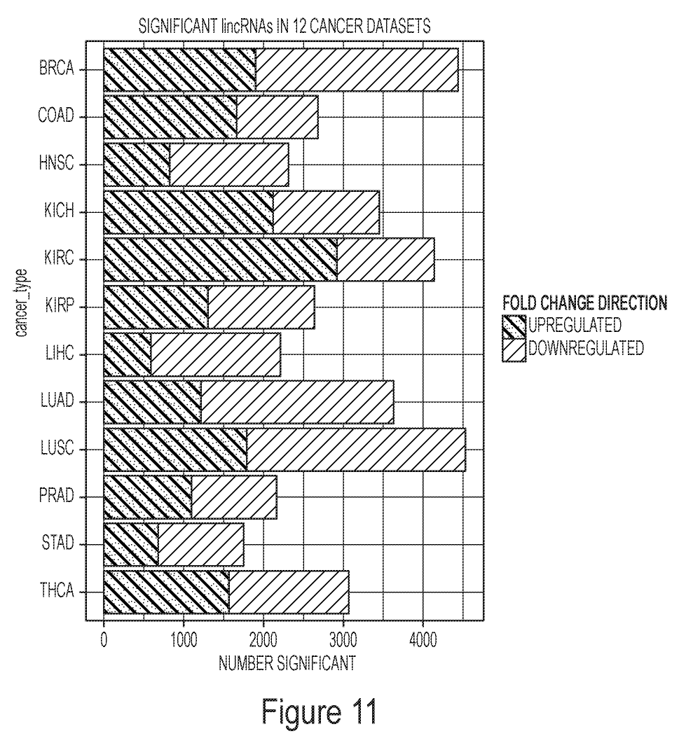

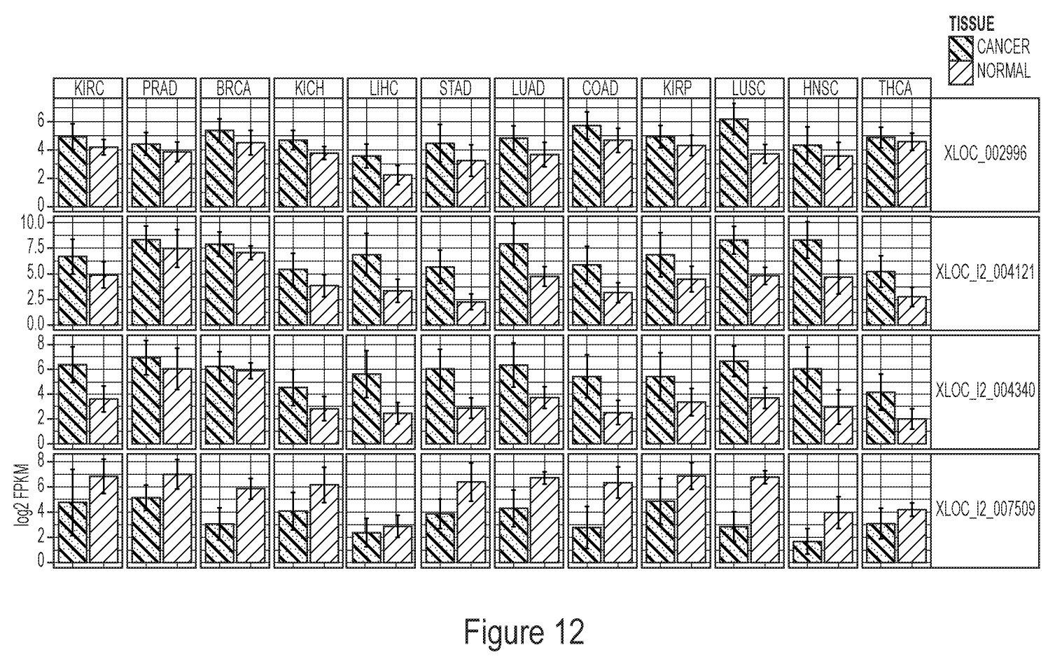

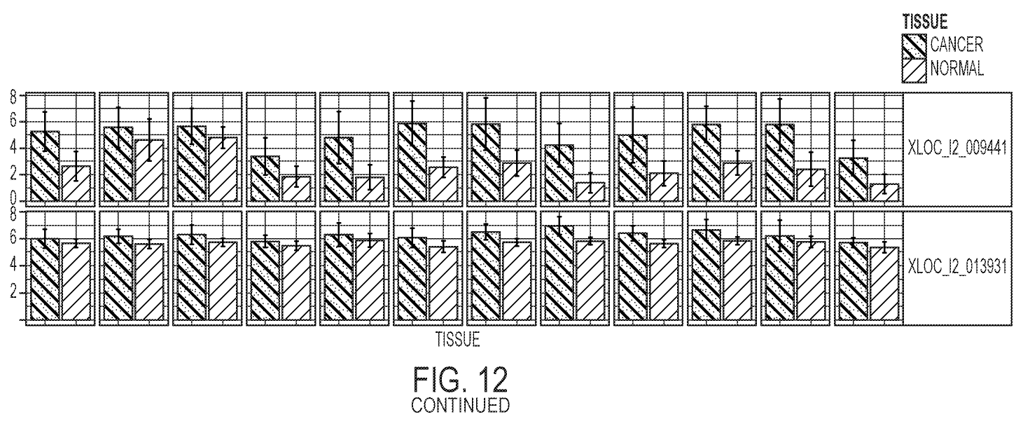

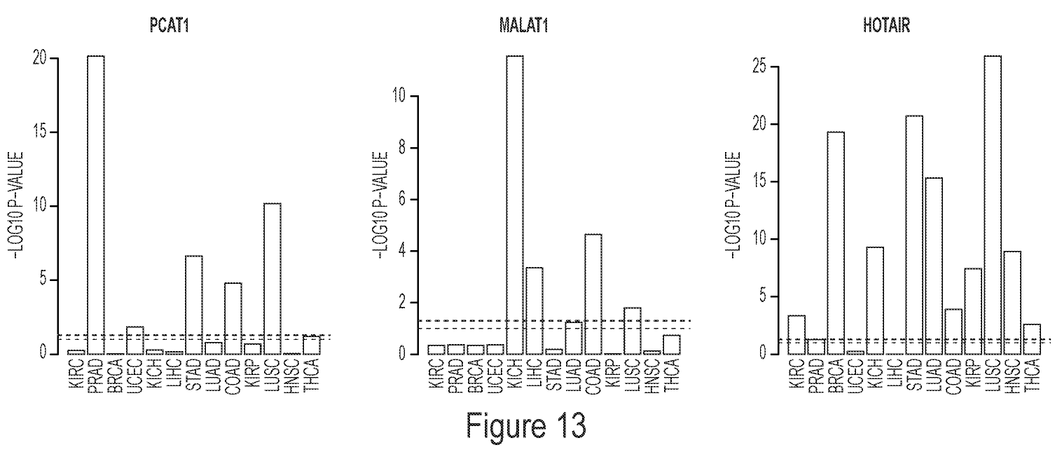

To seek a panel of lincRNAs as pan-cancer diagnostic biomarkers, differential expression analysis was performed on the above 12 TCGA datasets and detected thousands of differentially expressed lincRNAs in each TCGA dataset (FIG. 11). Among them, six lincRNAs are consistently and significantly altered in all 12 cancers, with five of them being up-regulated and one down-regulated (FIG. 4A, FIG. 12 and Table 3). On the contrary, when the same selection criteria was applied to protein coding genes, 47 mRNAs were identified. The much larger number of mRNAs is presumably due to the less tissue specificity of mRNAs and more annotated mRNAs compared to lincRNAs at the time of investigation.

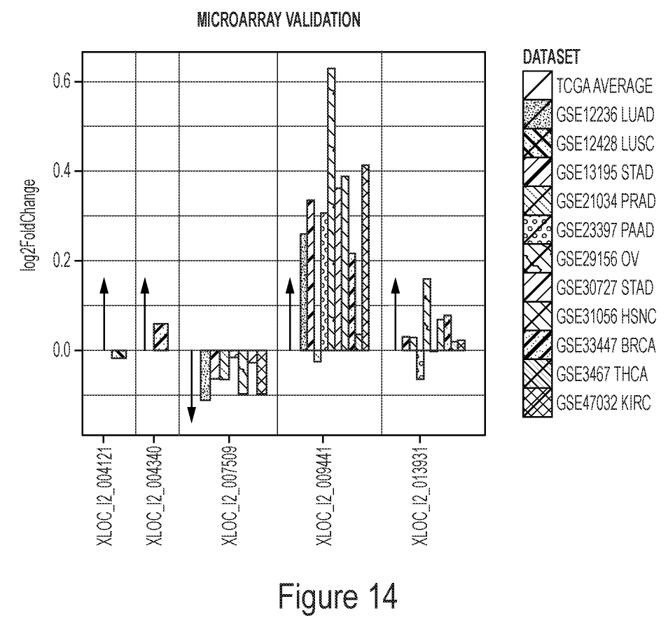

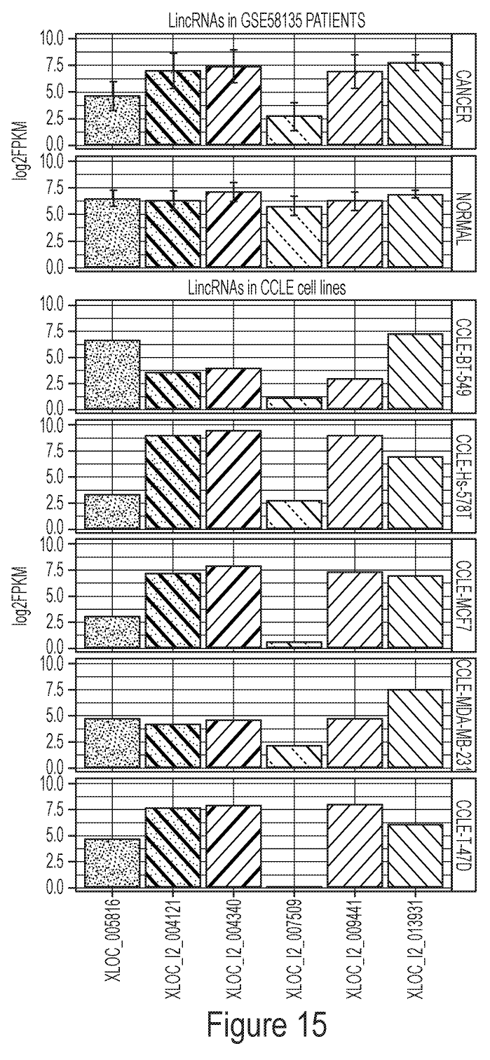

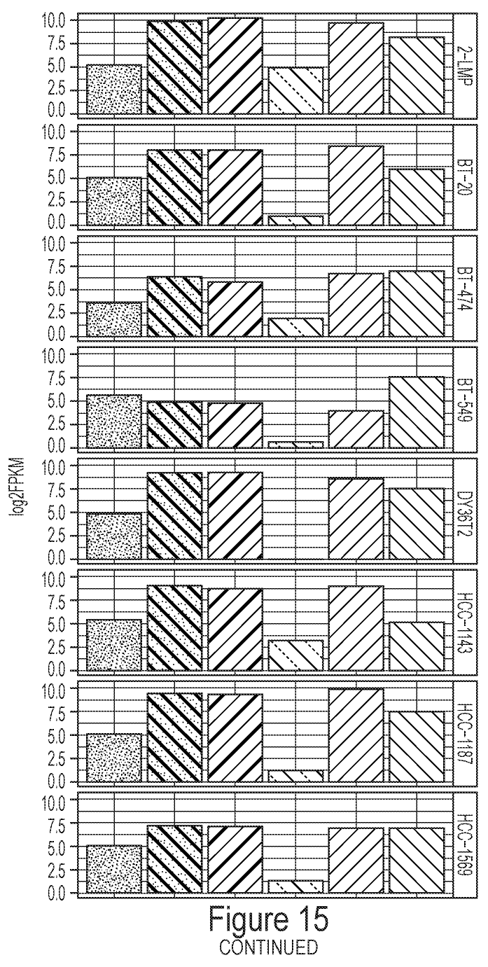

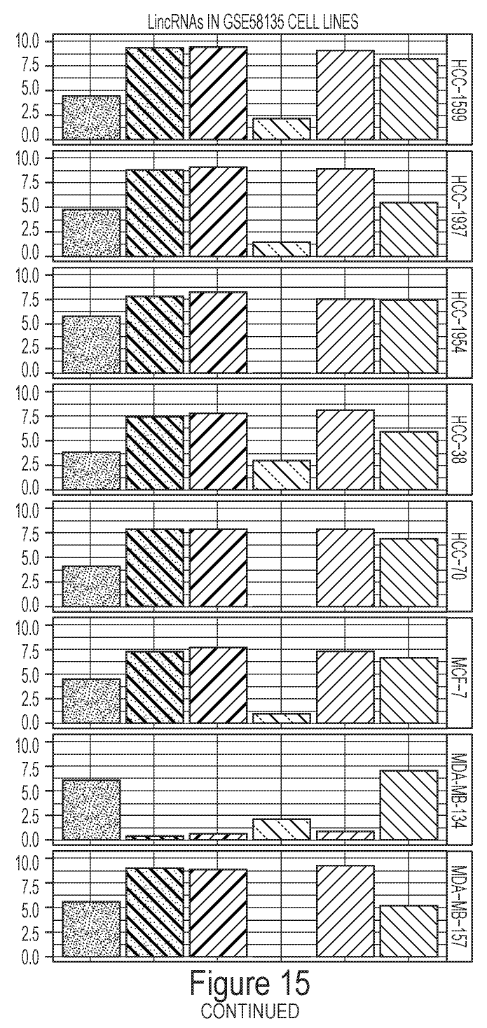

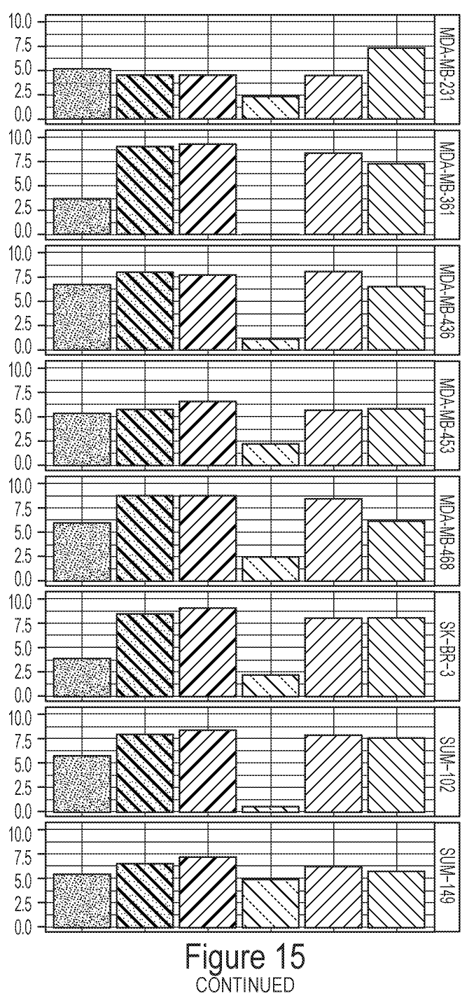

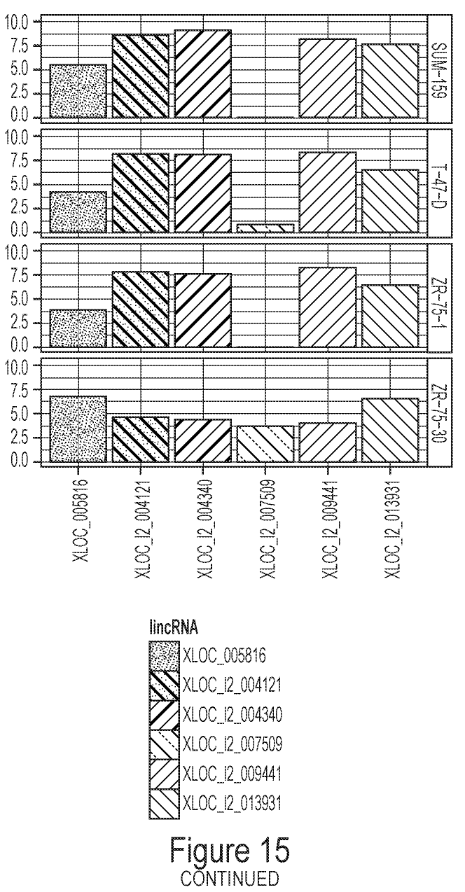

To confirm that the six lincRNAs are indeed associated with pan-cancers, an additional 833 samples were processed from a wide range of resources including three public RNA-Seq datasets and eleven microarray datasets (Table 1). All three public RNA-Seq datasets (GSE58135 breast cancer, GSE50760 colon cancer, and GSE25599 liver cancer) show consistent directions of fold change for all six lincRNAs (FIG. 4B). Although the microarray platforms are not designed to detect lincRNAs, some probes are nevertheless overlapped with non-coding RNAs as shown by others, and thus they can be another source of empirical verification. Among the various microarray platforms examined, 24 of the 29 microarray probe sets have the same overall directions of fold changes as those in the RNA-Seq datasets (FIG. 14). Moreover, the expression levels of the six lincRNAs in 28 breast cancer cell lines from the GSE58135 dataset and 5 breast cancer cell lines from the Cancer Cell Line Encyclopedia (CCLE) are all comparable with those from the TCGA BRCA samples (FIG. 15), further supporting the robustness of these lincRNAs as potential pan-cancer biomarkers.

To verify this lincRNA panel experimentally, additional RNA-Seq and qPCR experiments were performed on our own breast cancer samples. First, fresh frozen primary tumor samples from 10 individual patients were sequenced using the ribosomal depletion RNA-Seq method. These were then compared to normal breast tissue RNA-Seq data from GEO (GSE52194, GSE45326 and GSE30611). All six lincRNAs have the same trends of changes as in the other GEO RNA-Seq datasets (FIG. 4C) and five of them are significantly differentially expressed. The qPCR validation was used to follow-up and seven PCR primer pairs were designed for selected transcripts in the lincRNA panel (Table 4). The qPCR results in pooled breast tumor samples (n=5), pooled normal breast samples (n=5) and MCF-7 cell lines are shown in FIG. 4D. In all cases, the expression levels show statistically significant differential expression in the same directions as the RNA-Seq data, both between primary tumor and normal sample pools and between normal and MCF-7 cancer cell lines.

### Sequence Features Among the Six-lincRNA Biomarkers

To confirm the non-coding nature of the lincRNA transcripts, the iSeeRNA (Sun et al., 2013) and Coding-Potential Assessment Tool (CPAT) (Wang et al., 2013) was used. Both programs are specifically trained on long non-coding RNAs to assess the non-coding potential of RNA transcripts. Out of the 52 isoforms from the lincRNA panel, iSeeRNA predicted 49 to be non-coding. For the three transcripts that are ambiguous, a second tool, CPAT, was used to obtain further evidence for the coding or non-coding nature of these transcripts. CPAT classifies all three of them as non-coding RNAs. In contrast, both CPAT and iSeeRNA correctly classified all isoforms of house-keeping genes GUS and GAPDH as protein coding. Overall, both programs provide strong evidence for the non-coding nature of the six lincRNAs (Table 5).

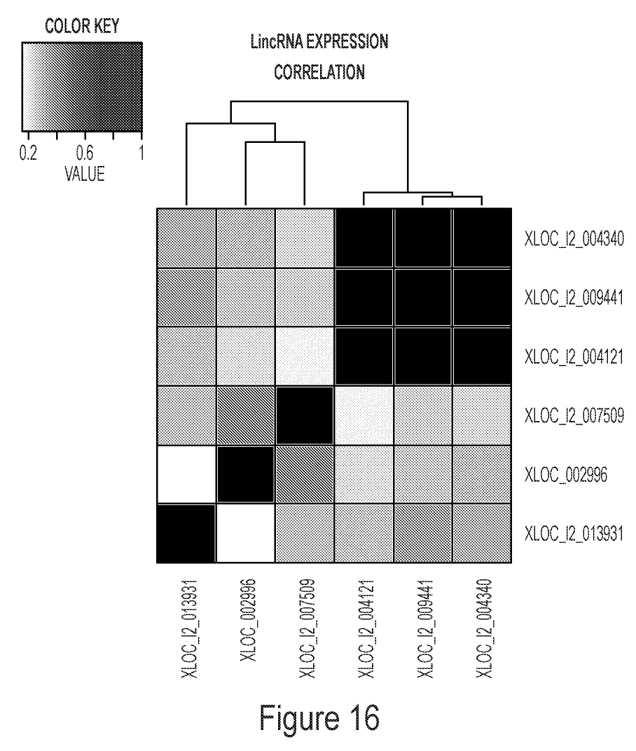

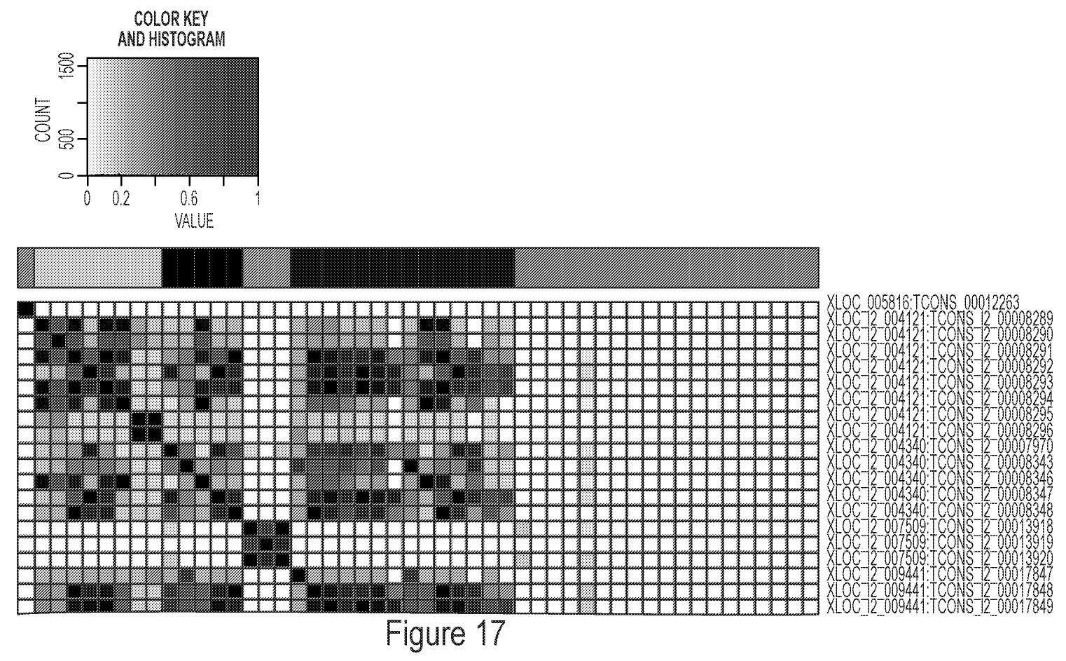

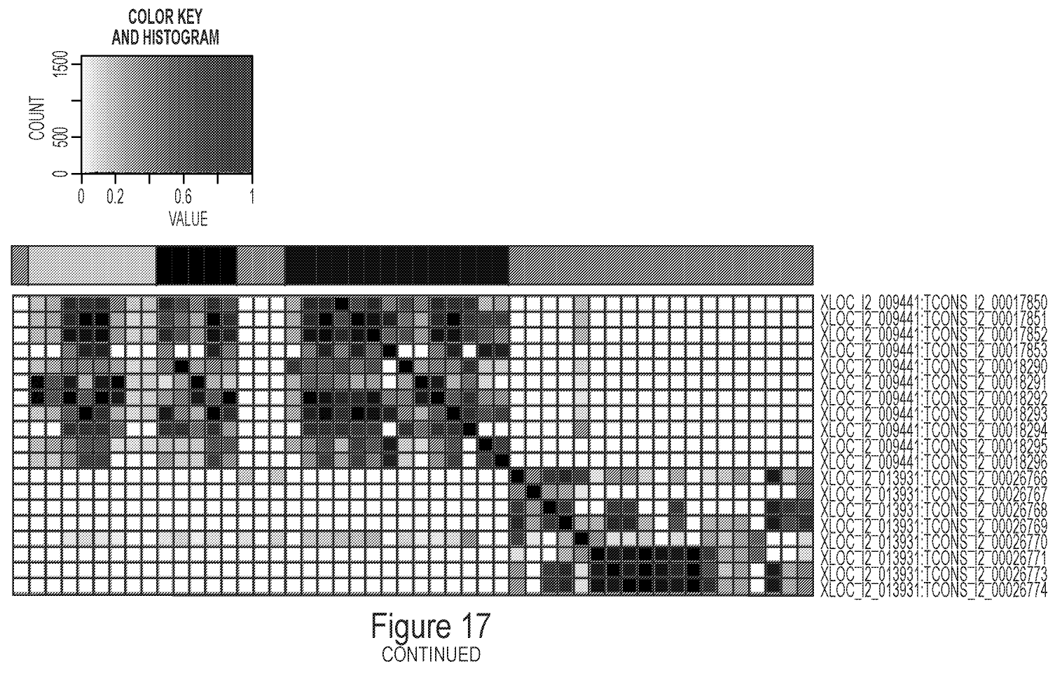

To examine the relationship between the six lincRNAs, the correlations of their expression values in all TCGA samples were first checked. Three of the lincRNAs, XLOC_12_004121, XLOC_12_004340 and XLOC_12_009441, are highly correlated with spearman correlation coefficients of approximately 0.92 between them (FIG. 16). The high correlations among expression prompted us to check if sequence similarities exist. Thus, the pairwise homology among all transcripts of the six lincRNAs was tested, using NCBI's BLAST+ suite (Camacho et al., 2009) (FIG. 17). Indeed, the three lincRNAs mentioned above are highly homologous, and some of the annotated transcripts are 99% identical. Two of the lincRNAs, XLOC_12_004121 and XLOC_12_004340, are in the tandem locations on chromosome 14 and the third lincRNA XLOC_12_009441 is located on chromosome 22, suggesting potential gene duplication events from a common origin.

The lincRNA Biomarker Panel Robustly and Accurately Predicts Pan Cancers

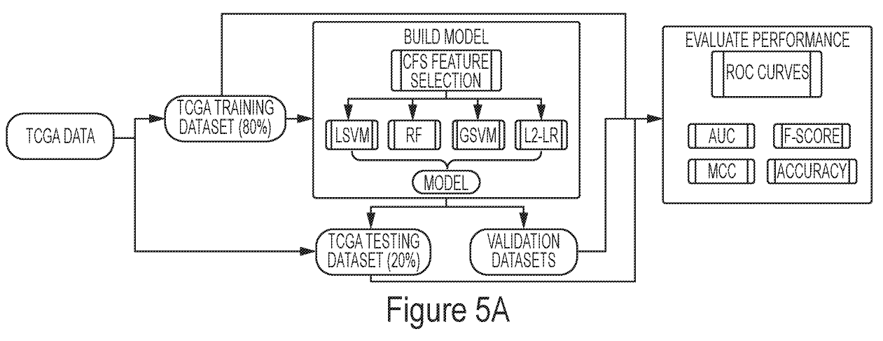

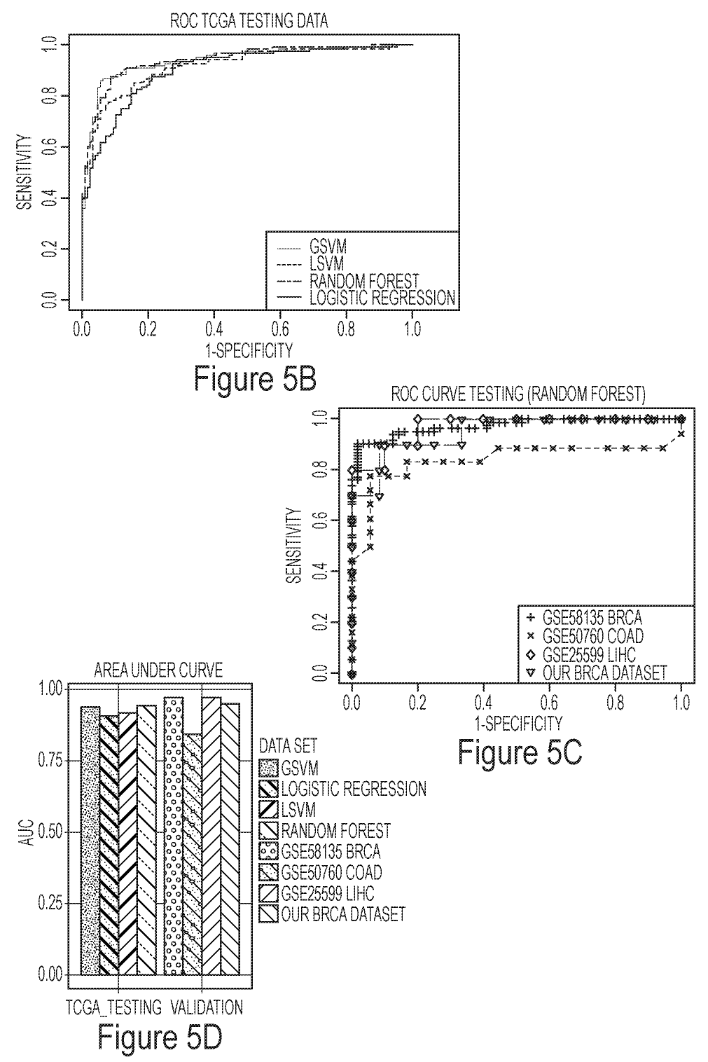

To quantitatively assess the value of the six lincRNAs as pan-cancer diagnostic biomarkers, a classification model was built upon them (FIG. 5A). First, the TCGA pan-cancer data was spilt into 80% training and 20% holdout testing sets. Given that some lincRNAs are highly correlated (FIG. 16) and thus potentially redundant as biomarker predictors, correlation feature selection (CFS) method was used to select the most relevant and least redundant subset of lincRNAs among them. As a result, five of the lincRNAs were chosen: XLOC_002996, XLOC_12_013931, XLOC_12_007509, XLOC_12_004121, and XLOC_12_004340.

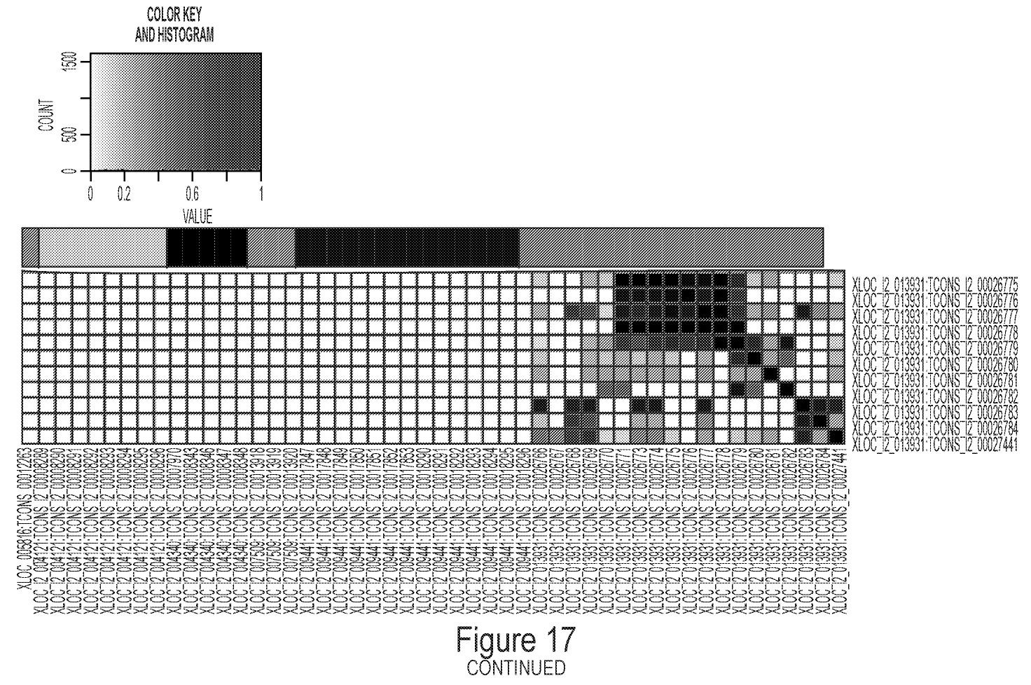

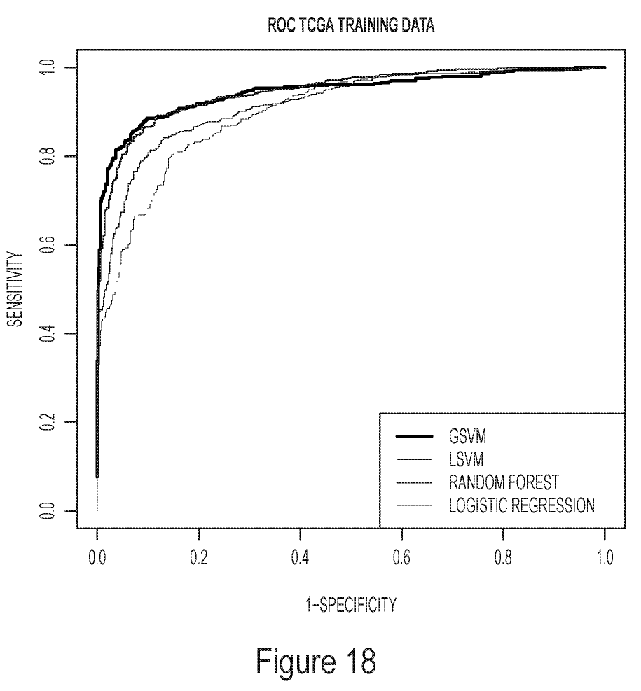

The classification results on the training dataset were then compared using four widely used machine-learning algorithms: Random Forest (RF), Linear Support Vector Machines (LSVM), Gaussian Support Vector Machines (GSVM) and Logistic Regression with L2 regularization (L2-LR). As shown by the receiver operator characteristics (ROC) curves on the TCGA training data set, RF has the best AUC of 0.947 (95% confidence interval, or CI: 0.9343-0.9603) on the training data among the four methods (FIG. 18). The RF model was thus selected to test the classification performance on additional 496 samples from the hold out test set. As expected, the trained RF model has very similar prediction result on the TCGA hold-out testing set, with an AUC=0.947, sensitivity=0.817 and specificity=0.970 (FIG. 5D).

To further verify the robustness of the five-lincRNA panel, the TCGA data based RF model was tested on four independent RNA-Seq datasets: GSE58135 breast cancer, GSE50760 colon cancer, GSE25599 liver cancer and our breast cancer dataset (FIGS. 5B, C and D). Impressively, this model predicts the other four independent data sets very well, with AUCs of 0.972 (95% CI: 0.95-0.9946), 0.841 (95% CI: 0.6875-0.9946), 0.970 (95% CI: 0.9108-1) and 0.950 (95% CI: 0.867-1) for GSE58135, GSE50760, GSE25599 and our dataset, respectively (FIGS. 5C and D). Other model evaluation metrics including Sensitivity, Specificity, Precision, Matthew's Correlation Coefficient, F-score and Accuracy in the validation datasets further demonstrate the excellent performance of the model (Table 6). It was therefore concluded that the panel of six lincRNAs are potential biomarkers for pan-cancer diagnosis.

The lincRNA Panel is Associated with Prognosis in Cancer Patients

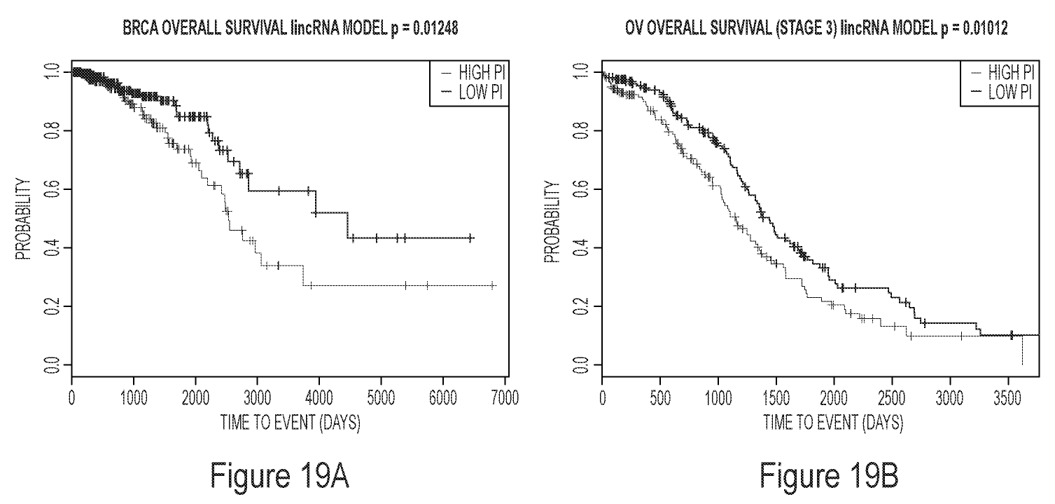

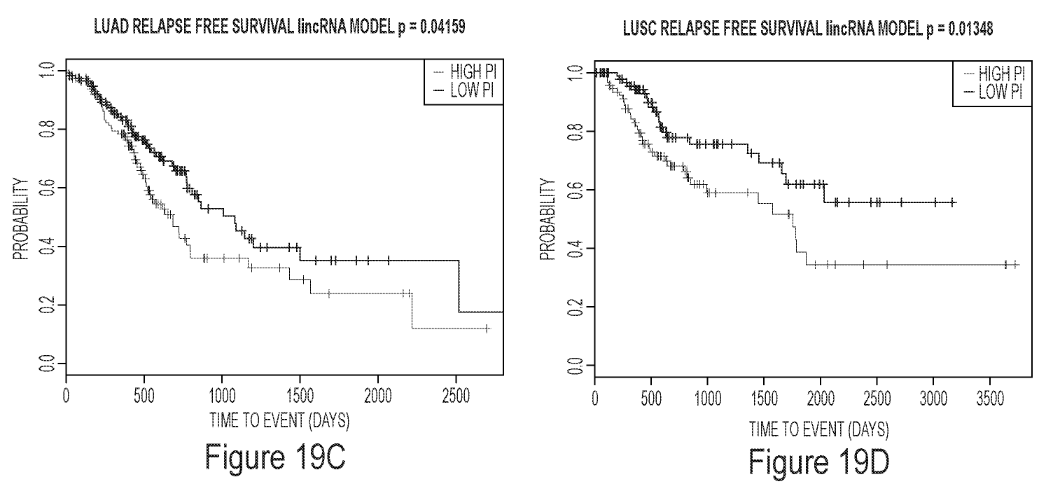

Although the six lincRNAs were detected as potential diagnosis markers for pan-cancer, whether they might be associated with the prognosis of cancer patients as well was investigated. Thus, survival analysis was performed on 1201 samples from four TCGA datasets: namely BRCA, LUAD, LUSC datasets, and additionally the TCGA ovarian cancer (OV) dataset which was not used in the lincRNA signature discovery phase due to lack of normal samples (FIGS. 19A-D). Since only overall survival information is available in TCGA in BRCA and OV datasets, the overall survival was fitted with Cox-PH regression models and the patient risks by prognosis index (PI)(Huang et al., 2014) was categorized. The resulting Kaplan-Meier survival curves show that the lincRNA panel is able to separate patients into higher and lower risk groups by median PI, with log-rank tests p-values of 0.012 and 0.010 for BRCA and grade 3 OV, respectively (FIGS. 19a and b). On the other hand, the more preferable relapse free survival (RFS) in LUAD and LUSC datasets are available, thus RFS was fitted with Cox-PH models, and obtained significant p-values of 0.0416 and 0.013 for differential survivals of LUAD and LUSC samples, respectively (FIGS. 19C and D). In summary, although the lincRNA panel was not purposely discovered as prognosis markers but rather diagnostic markers, their expression values are associated with the prognosis outcomes in various types of cancers.

Biological Relevance of lincRNAs Explored by Cell Culture Experiments

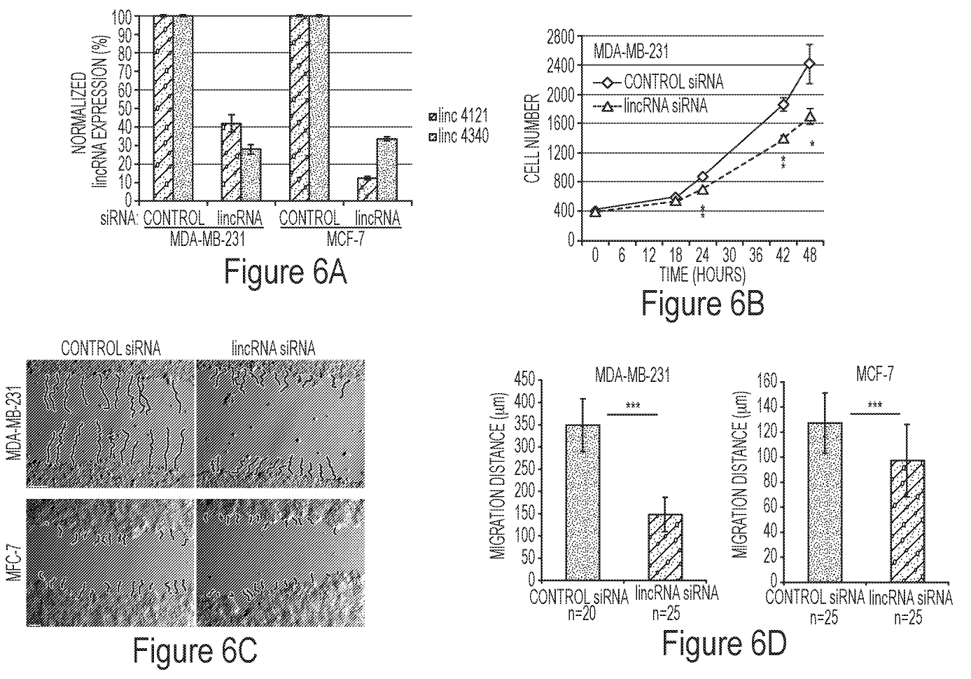

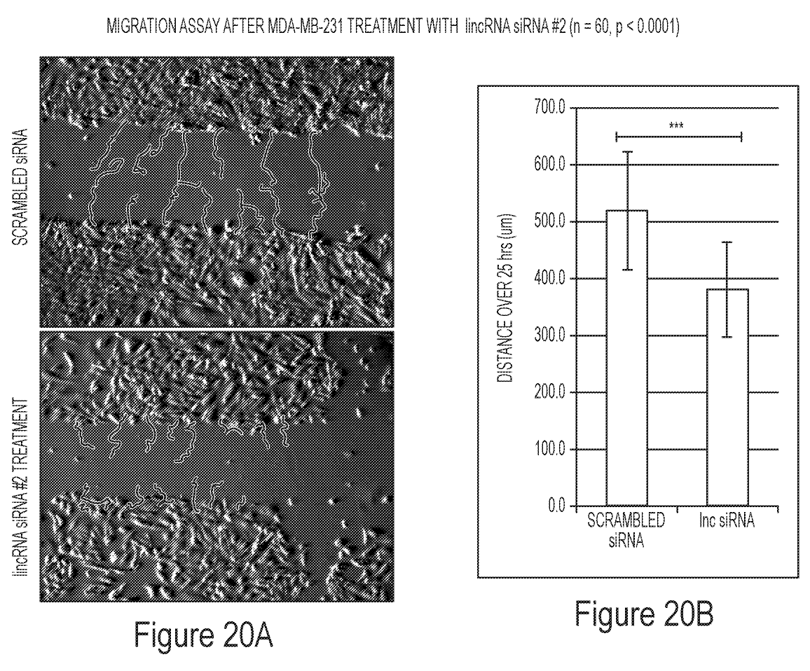

To explore the relationship between the lincRNAs panel and tumorigenic phenotypes, experiments were conducted using two breast cancer and colon cancer cell lines as examples. Given the extremely high homology between XLOC_12_004121 and XLOC_12_004340, siRNAs were specifically designed that target both of them so as to observe phenotypes. In non-aggressive MCF-7 and highly metastatic MDA-MB-231 cell lines, two lincRNAs XLOC_12_004121 and XLOC_12_004340 (FIG. 6A) were efficiently knocked down. Transient knockdown allowed us to analyse cell proliferation and cell migration rate. Interestingly, the growth rate of fast proliferating MDA-MB-231 cells significantly decreased upon transfection with lincRNAs siRNA (FIG. 6B). To assess cell migration rates the well-established wound-healing assay was employed and the cell movement with time-lapse microscopy over the time of 24 hours was followed. As expected, the migration rate was significantly inhibited upon lincRNAs knockdown (FIG. 6C, D). The effect of lincRNA down-regulation on cell migration was more pronounced in a highly aggressive MDA-MB-231 cell line (0.349 versus 0.059 mm over 24 hours for control and lincRNA siRNA, respectively) but it was also observed in much slower migrating MCF-7 cells (0.127 versus 0.096 mm over 24 hours for control and lincRNA siRNA, respectively). The cell migration experiment was repeated on MDA-MB-231 with another less effective siRNA, and observed similar significantly slower (P<0.0001) migrating rate (FIGS. 20A-B).

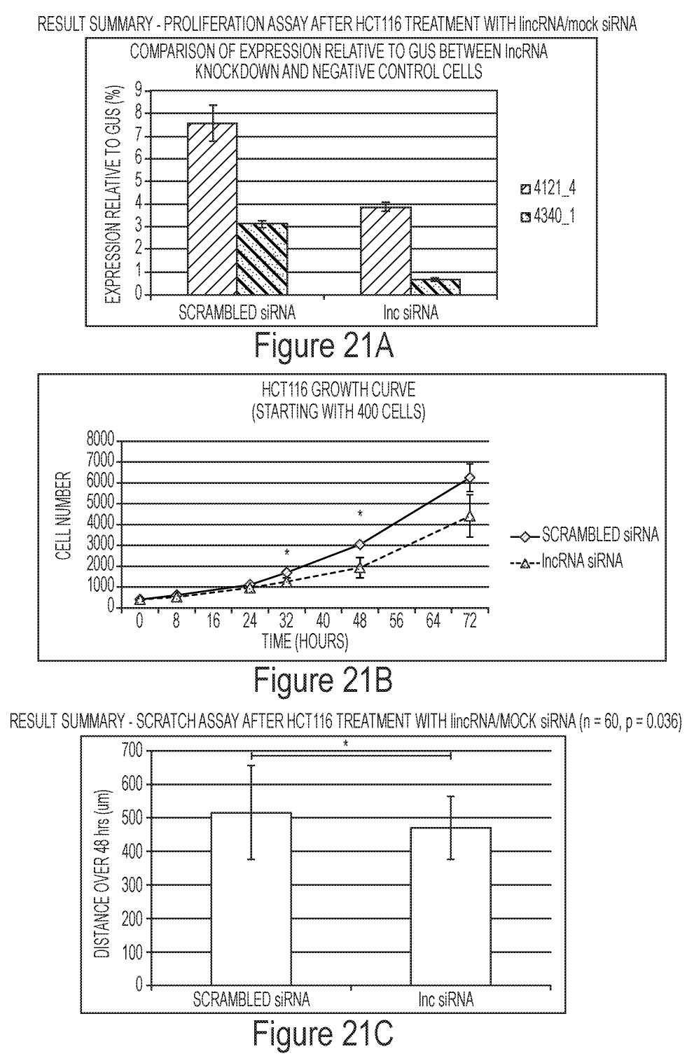

Furthermore, these experiments were repeated in another HCT116 colon cancer cell line with the more efficient siRNA (FIGS. 21A-C). Using the same experimental procedures, significant differences were observed in both cell proliferation (p<0.0001) and migration (p=0.036), between the lincRNA knockdown and the siRNA scrambled control. These results suggest that down-regulation of cancer cell abundant XLOC_12_004121 and XLOC_12_004340 lincRNAs weakens the typical cancer phenotypic features, such as proliferation and migration.

### Discussion

Since 2012, a community effort has launched towards TCGA pan-cancer analysis across many different tumor types, where the main focus has been the mutational landscape. Pan-Cancer Initiative aims to enable the discovery of novel intervention strategies that can be tested clinically, including developing novel adaptive biomarker-based clinical trials that cross boundaries between tumor types (Cancer Genome Atlas Research et al., 2013). One can expect that in the future, a pan-cancer screening biomarker panel from blood or other body fluids could become a useful, routine, and economical screening tool (Cancer Genome Atlas Research et al., 2013) applied before the patients have typical cancer symptoms that indicate late-stage character of the disease. Once an individual is identified as high-risk in the test, he or she can be followed up with more confirmative tests, such as imaging scanning. The clinical potential of lincRNAs remains under-explored across different tumor types. In this study, the goals were to (1) depict the landscape of lincRNAs in pan-cancers, (2) demonstrate their relevance to clinical outcomes, such as tumor subtype, diagnosis and patient survival; and (3) explore the utilities of lincRNAs as pan-cancer diagnostic biomarkers.

Towards these goals, a new dimension of pan-cancer analysis using the lincRNA transcriptome was performed. In total, 3354 patient RNA-Seq samples from 12 types of cancers in TCGA (13 including OV in survival analysis), as well as an additional 15 independent datasets (three RNA-Seq datasets from GEO, one in-house RNA-Seq breast cancer dataset and 11 microarray datasets from GEO) were analyzed. To our knowledge, this study is the most comprehensive endeavour to analyze lincRNAs in the context of pan-cancer. By systematically analyzing 12 types of RNA-Seq datasets in TCGA, it was shown that lincRNAs are more tissue specific than protein-coding genes. The loss of tissue specificity due to cancer is greater for lincRNAs compared to protein-coding genes. This suggests that lincRNAs can potentially be more sensitive biomarkers than protein coding genes. In addition, unsupervised clustering results of lincRNAs demonstrate significant correlations with molecular subtypes. CNMF clustering based on lincRNAs almost perfectly divided the Triple Negative and ER+/Her2− breast cancers into distinct groups in GSE58135 data set. Furthermore, CNMF clustering of TCGA BRCA samples detected 5 distinct clusters that highly correspond to the five widely used molecular subtypes based on the PAM50 signatures.

A promising six-lincRNA pan-cancer diagnostics panel quantitatively was pinpointed, rigorously and robustly. Moreover, the alteration of these lincRNAs was verified with eleven additional microarray gene expression data sets. The most unexpected finding is that the six lincRNA diagnostic signature is also associated with the survival prognosis of cancer patients, based on the TCGA datasets (BRCA, OV, LUAD and LUSC). Furthermore, it has been demonstrated that the lincRNAs have biological functions, by knocking-down experiments on two of them, XLOC_12_004121 and XLOC_12_004340. These preliminary results indicate that downregulation of only two out of six panel lincRNAs is sufficient to partially revert some of the typical physiological hallmarks of cancer cells including fast proliferation and more importantly, migration.

Developing a pan-cancer biomarker model based on the lincRNA signatures could be very significant clinically, providing complementary values to protein-coding gene based biomarker panels. Although lincRNAs do not encode proteins, it's clear that they play important roles in cellular biology. Currently, multiple hypotheses exist on how lincRNAs regulate cellular functions, which include functioning as scaffold structure, sponge of small regulatory RNAs or direct interaction with proteins to modulate localization and activity. To better understand the phenotypic effects of the six lincRNAs, as well as molecular mechanisms by which they promote tumorigenesis and/or malignancy, experiments that address the physiological functions of these lincRNAs may be performed.

In summary, this initial pan-cancer analysis has demonstrated that lincRNAs accurately classify cancer subtypes through supervised as well as unsupervised methods. The panel of six lincRNAs is a highly accurate diagnostic biomarker signature with additional prognostic value. These results highlight lincRNAs as a new paradigm for actionable pan-cancer diagnosis and prognosis.

TABLE 1
Tabulation of the patients, tumor samples and normal adjacent tissue samples used in this study.

| TCGA datasets | | | | | |
|---|---|---|---|---|---|
| | Primary | Adjacent | | # mapped | |
| Dataset | Tumor | Normal | Paired | reads | |
| Breast invasive carcinoma | 1059 | 111 | 111 | 193560468 | |
| Colon adenocarcinoma | 444 | 41 | 41 | 44872617 | |
| Head and Neck squamous cell carcinoma | 498 | 43 | 41 | 57516555 | |
| Kidney Chromophobe | 66 | 25 | 25 | 42982445 | |
| Kidney renal clear cell carcinoma | 531 | 72 | 72 | 94701140 | |
| Kidney renal papillary cell carcinoma | 226 | 32 | 32 | 48960010 | |
| Liver hepatocellular carcinoma | 210 | 50 | 50 | 46819687 | |
| Lung adenocarcinoma | 489 | 58 | 57 | 69621821 | |
| Lung squamous cell carcinoma | 489 | 50 | 50 | 86702630 | |
| Prostate adenocarcinoma | 419 | 52 | 52 | 98002347 | |
| Stomach adenocarcinoma | 285 | 32 | 30 | 65146435 | |
| Thyroid carcinoma | 498 | 59 | 59 | 127022122 | |
| Subtotal | 5214 | 625 | 620 | 975908277 | |
| Samples used for survival analysis | Cancer | | | | |
| Breast invasive carcinoma | 463 | | | | |
| Ovarian serous adenocarcinoma | 406 | | | | |
| Lung adenocarcinoma | 193 | | | | |
| Lung squamous cell carcinoma | 139 | | | | |
| Samples used for subtype analysis | Cancer | | | | |
| Breast invasive carcinoma | 521 | | | | |
| Validation Datasets | | | | | |
| Accession | Cancer | Non-cancer | | | Additional Details |
| GSE25599 | 10 | 10 | | | Single end, 36 bp, Liver Cancer (HBV) |
| GSE58135 | 84 | 56 | | | Paired, 100 bp, Breast Cancer |
| GSE50760 | 36 | 36 | | | Paired, 100 bp, Colon cancer |
| Our BRCA dataset | 10 | 6 | | | Single end, 100 bp, BRCA; normal samples: |
| | | | | | GSE52194, GSE45326, GSE30611 |
| Whole Human Genome Oligo Microarray G4112A | Cancer | Non-cancer | | | Additional Details |
| GSE12428 | 34 | 28 | | | Microarray, lung squamous cell carcionoma |
| Affymetrix Human Exon 1.0 ST Array | Cancer | Non-cancer | | | Additional Details |
| GSE30727 | 30 | 30 | | | Microarray, stomach cancer |
| GSE47032 | 20 | 20 | | | Microarray, kidney clear cell cancer |
| GSE23397 | 15 | 6 | | | Microarray, pancreatic cancer |
| GSE21034 | 150 | 29 | | | Microarray, prostate cancer |
| GSE13195 | 25 | 25 | | | Microarray, stomach cancer |
| GSE12236 | 16 | 24 | | | Microarray, lung cancer |
| GSE29156 | 9 | 11 | | | Microarray, ovarian cancer |
| Affymetrix Human Genome U133 Plus 2.0 Array | Cancer | Non-cancer | | | Additional Details |
| GSE3467 | 9 | 9 | | | Microarray, thyroid samples |
| GSE31056 | 23 | 72 | | | Microarray, oral carcinoma |
| SurePrint G3 Human GE 8 × 60K Microarray | Cancer | Non-cancer | | | Additional Details |
| GSE33447 | 8 | 8 | | | Microarray, breast cancer samples |
| | | Triple | | | |
| | ER+/HER2− | Negative | | | |
| GSE58135 subtype analysis | 42 | 42 | | | |

| | |
|---|---|
| Total unique biological samples | 3354 |

TABLE 2
A list of tissue specific lincRNAs and their associated
tissue type, defined by JS score > 0.75 and
expression in at least 5% of the samples.

| lincRNA | JS Score | Associated Tissue |
|---|---|---|
| XLOC_010690 | 0.766204217 | kidney |
| XLOC_007596 | 0.857703592 | liver |
| XLOC_003732 | 0.752100476 | colon |
| XLOC_010399 | 0.768482652 | kidney |
| XLOC_008645 | 0.758646776 | lung |
| XLOC_011257 | 0.763064551 | liver |
| XLOC_001387 | 0.77810803 | liver |
| XLOC_007597 | 0.824089647 | liver |
| XLOC_000947 | 0.903394197 | adipose + breast |
| XLOC_011275 | 0.911613639 | liver |
| XLOC_004719 | 0.941948946 | kidney |
| XLOC_013795 | 0.781746482 | adipose + breast |
| XLOC_004102 | 0.873732325 | kidney |
| XLOC_004360 | 0.785572253 | adipose + breast |
| XLOC_003239 | 0.775039257 | lung |
| XLOC_008455 | 0.770565259 | adipose + breast |
| XLOC_004177 | 1 | lung |
| XLOC_001901 | 0.760514635 | liver |
| XLOC_004836 | 0.754396243 | adipose + breast |
| XLOC_004514 | 0.794355439 | liver |
| XLOC_004801 | 0.906048522 | adipose + breast |
| XLOC_004261 | 0.817042521 | adipose + breast |
| XLOC_000224 | 0.799618236 | kidney |
| XLOC_004269 | 0.867461193 | kidney |
| XLOC_008454 | 0.792515074 | adipose + breast |
| XLOC_004515 | 0.752963989 | liver |
| XLOC_005515 | 0.767317503 | thyroid |
| XLOC_008260 | 0.902906732 | lung |
| XLOC_001704 | 0.848966352 | liver |
| XLOC_013779 | 0.756769238 | head + neck |
| XLOC_007883 | 0.926395141 | liver |
| XLOC_006805 | 0.772086177 | kidney |
| XLOC_008446 | 1 | kidney |
| XLOC_003315 | 0.791869141 | colon |
| XLOC_012693 | 0.755588854 | liver |
| XLOC_000857 | 0.760980892 | kidney |
| XLOC_009690 | 0.763803004 | lung |
| XLOC_001425 | 0.900101922 | kidney |
| XLOC_005607 | 0.808412113 | liver |
| XLOC_010419 | 0.779716586 | thyroid |
| XLOC_013465 | 0.754345459 | prostate |
| XLOC_007017 | 0.773648455 | adipose + breast |
| XLOC_008602 | 0.767872806 | liver |
| XLOC_007405 | 0.804291982 | prostate |
| XLOC_014355 | 0.762921632 | prostate |
| XLOC_013037 | 0.768622905 | prostate |
| XLOC_001378 | 0.862077396 | kidney |
| XLOC_005775 | 0.817266832 | kidney |
| XLOC_000430 | 0.936023257 | liver |
| XLOC_l2_000357 | 0.767464299 | lung |
| XLOC_l2_013883 | 0.914582666 | prostate |
| XLOC_l2_001548 | 0.763547727 | adipose + breast |
| XLOC_l2_004342 | 0.768931463 | prostate |

TABLE 3
Genomic coordinate descriptions of the six lincRNAs
and cross-reference with the Ensemble database.

| | Human Body | | lincRNA | lincRNA | lincRNA | lincRNA | Closest |
|---|---|---|---|---|---|---|---|
| lincRNA | Map ID | Chr. | strand | start | width | end | Gene |
| PCAN-1 | XLOC_002996 | chr3 | + | 195367106 | 10516 | 195377622 | APOD |
| PCAN-2 | XLOC_l2_004121 | chr14 | + | 19650018 | 45163 | 19695181 | POTEG |
| PCAN-3 | XLOC_l2_004340 | chr14 | − | 19856361 | 68973 | 19925334 | POTEM |
| PCAN-4 | XLOC_l2_007509 | chr2 | + | 114298969 | 29137 | 114328106 | FOXD4L1 |
| PCAN-5 | XLOC_l2_009441 | chr22 | − | 16101370 | 91857 | 16193227 | POTEH |
| PCAN-6 | XLOC_l2_013931 | chr7 | − | 97503667 | 98000 | 97601667 | ASNS |

| | | Gene | Gene | Gene | Ensembl | Ensembl |
|---|---|---|---|---|---|---|
| lincRNA | Distance | start | width | end | Annotation | gene match |
| PCAN-1 | 56029 | 195295573 | 15504 | 195311077 | LincRNA, | N/A |
| | | | | | Unannotated | |
| PCAN-2 | 65075 | 19553365 | 31578 | 19584943 | LincRNA, | AL589743.1 |
| | | | | | Unannotated | |
| PCAN-3 | 58620 | 19983954 | 36319 | 20020273 | LincRNA, | CTD- |
| | | | | | Unannotated | 2314B22.3 |
| PCAN-4 | 40241 | 114256661 | 2067 | 114258728 | Processed | N/A |
| | | | | | Pseudogene, | |
| | | | | | Unannotated | |
| PCAN-5 | 63105 | 16256332 | 31606 | 16287938 | LincRNA, | AP000525.9 |
| | | | | | Processed | |
| | | | | | Transcript, | |
| | | | | | Unannotated | |
| PCAN-6 | 1812 | 97481429 | 20426 | 97501855 | Processed | N/A |
| | | | | | Pseudogene, | |
| | | | | | Unannotated | |

*Ensembl v75 (GRCh37), annotated exons

TABLE 4
Primer designs for quantitative real-time PCR of
the lincRNA panel.

| Assay | LincRNA | |
|---|---|---|
| Label | target | Primers |
| 4121_1: | PCAN-2 | F: 5′-AGCTTCGGAGAAGCAGTGGT-3′ |
| | (XLOC_12_ | (SEQ ID NO: 7) |
| | 004121) | R: 5′-TTCTTTCCGCGGAGACCT-3′ |
| | | (SEQ ID NO: 8) |
| 4340_4 | PCAN-3 | F: 5′-ACAGATGAACCGCGGAGAC-3′ |
| | (XLOC_12_ | (SEQ ID NO: 9) |
| | 004340) | R: 5′-AGCTTCGGAGAAGCAGTGGT-3′ |
| | | (SEQ ID NO: 7) |
| 2996_3 | PCAN-1 | F: 5′-TAAGGGTCATGGAGCTGGAG-3′ |
| | (XLOC_12_ | (SEQ ID NO: 10) |
| | 002996) | R: 5′-ATCAGCTCCTCCCCGAGTAT-3′ |
| | | (SEQ ID NO: 11) |
| 7509_4 | PCAN-4 | F: 5′-GAAGTTTAATGTTGCCAATGGA-3′ |
| | (XLOC_12_ | (SEQ ID NO: 12) |
| | 007509) | R: 5′-GCCTTTGCACAGACTGACCT-3′ |
| | | (SEQ ID NO: 13) |
| 13931_6 | PCAN-6) | F: 5′-ATCCAGAACTGCAGCCAGTC-3′ |
| | (XLOC_12_ | (SEQ ID NO: 14) |
| | 013931) | R: 5′-AGAAGTACATGGGGGTGTGG-3′ |
| | | (SEQ ID NO: 15) |

Tables 5A-B. Coding potential predictions of all isoforms of the lincRNA panel, using iSeeRNA (http://137.189.133.71/iSeeRNA/) and Coding Potential Assessment Tool (CPAT) (http://lilab.research.bcm.edu/cpat/calculator_sub.php). Additional positive controls using protein-coding genes GAPDH and GUS are also listed.

TABLE 5A
CPAT Results.

| | RNA | ORF | Ficket | Hexamer | Coding | Coding |
|---|---|---|---|---|---|---|
| Sequence Name | Size | Size | Score | Score | Probability | Label |
| PCAN-1 (XLOC_002996) | | | | | | |
| TCONS_00006377 | 6659 | 633 | 0.5291 | 0.173947044 | 0.760142576 | yes |
| TCONS_00006378 | 1716 | 135 | 1.0111 | 0.171563902 | 0.080769211 | no |
| TCONS_00007073 | 211 | 96 | 0.6124 | −0.376596856 | 0.000483433 | no |
| TCONS_00007074 | 1586 | 153 | 1.1382 | 0.213026381 | 0.17417223 | no |
| PCAN-2 (XLOC_l2_004121) | | | | | | |
| TCONS_L2_00008289 | 768 | 117 | 0.7464 | 0.041918727 | 0.013971842 | no |
| TCONS_L2_00008290 | 549 | 192 | 0.8298 | 0.048534387 | 0.042017289 | no |
| TCONS_L2_00008291 | 2102 | 276 | 0.484 | −0.012649119 | 0.021810528 | no |
| TCONS_L2_00008292 | 1406 | 327 | 0.639 | −0.180683866 | 0.021204847 | no |
| TCONS_L2_00008293 | 2391 | 267 | 0.42 | −0.077650944 | 0.010473072 | no |
| TCONS_L2_00008294 | 4116 | 267 | 0.42 | −0.077650944 | 0.009441453 | no |
| TCONS_L2_00008295 | 1060 | 342 | 0.5027 | −0.119244657 | 0.02480705 | no |
| TCONS_L2_00008296 | 990 | 342 | 0.5027 | −0.119244657 | 0.024910079 | no |
| PCAN-3 (XLOC_l2_004340) | | | | | | |
| TCONS_L2_00007970 | 5950 | 459 | 0.6599 | 0.112352222 | 0.336164826 | no |
| TCONS_L2_00008343 | 1169 | 144 | 0.6051 | −0.107657632 | 0.004404995 | no |
| TCONS_L2_00008346 | 4142 | 267 | 0.42 | −0.077650944 | 0.009426701 | no |
| TCONS_L2_00008347 | 1405 | 327 | 0.639 | −0.180683866 | 0.021206108 | no |
| TCONS_L2_00008348 | 570 | 168 | 0.904 | −0.068320034 | 0.019342915 | no |
| PCAN-4 (XLOC_l2_007509) | | | | | | |
| TCONS_L2_00013918 | 2879 | 270 | 0.6819 | −0.343879747 | 0.004149952 | no |
| TCONS_L2_00013919 | 547 | 192 | 1.0539 | 0.129150962 | 0.131216094 | no |
| TCONS_L2_00013920 | 2179 | 270 | 0.6819 | −0.343879747 | 0.00432936 | no |
| PCAN-5 (XLOC_l2_009441) | | | | | | |
| TCONS_L2_00017847 | 609 | 150 | 0.8864 | −0.136597695 | 0.009670352 | no |
| TCONS_L2_00017848 | 880 | 168 | 0.868 | −0.07522081 | 0.016248197 | no |
| TCONS_L2_00017849 | 913 | 168 | 0.868 | −0.07522081 | 0.016216201 | no |
| TCONS_L2_00017850 | 763 | 168 | 0.868 | −0.07522081 | 0.016362141 | no |
| TCONS_L2_00017851 | 757 | 144 | 0.6203 | −0.079415711 | 0.005701195 | no |
| TCONS_L2_00017852 | 631 | 168 | 0.868 | −0.07522081 | 0.016491637 | no |
| TCONS_L2_00017853 | 359 | 93 | 0.9437 | −0.087655495 | 0.008776281 | no |
| TCONS_L2_00018290 | 1174 | 144 | 0.6051 | −0.107657632 | 0.004403664 | no |
| TCONS_L2_00018291 | 4136 | 267 | 0.42 | −0.077650944 | 0.009430103 | no |
| TCONS_L2_00018292 | 2102 | 276 | 0.484 | −0.012649119 | 0.021810528 | no |
| TCONS_L2_00018293 | 1394 | 327 | 0.6533 | −0.197474682 | 0.019892986 | no |
| TCONS_L2_00018294 | 413 | 144 | 0.6051 | −0.107657632 | 0.004610957 | no |
| TCONS_L2_00018295 | 871 | 102 | 0.8213 | −0.089455015 | 0.006323578 | no |
| TCONS_L2_00018296 | 551 | 258 | 0.6655 | 0.081717204 | 0.062360503 | no |
| PCAN-6 (XLOC_l2_013931) | | | | | | |
| TCONS_L2_00026766 | 1676 | 459 | 0.484 | 0.100992688 | 0.259495691 | no |
| TCONS_L2_00026767 | 809 | 591 | 0.702 | 0.137974424 | 0.795810077 | yes |
| TCONS_L2_00026768 | 376 | 252 | 1.047 | 0.176429194 | 0.280760562 | no |
| TCONS_L2_00026769 | 453 | 153 | 1.0189 | 0.316429065 | 0.23511127 | no |
| TCONS_L2_00026770 | 4110 | 396 | 0.618 | −0.361689179 | 0.010864527 | no |
| TCONS_L2_00026771 | 1215 | 267 | 1.1441 | 0.363689952 | 0.67084369 | yes |
| TCONS_L2_00026773 | 1150 | 267 | 1.1441 | 0.363689952 | 0.671714583 | yes |
| TCONS_L2_00026774 | 1091 | 267 | 1.1441 | 0.363689952 | 0.672504065 | yes |
| TCONS_L2_00026775 | 829 | 267 | 1.1441 | 0.363689952 | 0.675998075 | yes |
| TCONS_L2_00026776 | 930 | 327 | 1.2265 | 0.349269552 | 0.823711195 | yes |
| TCONS_L2_00026777 | 1119 | 267 | 1.1441 | 0.363689952 | 0.672129517 | yes |
| TCONS_L2_00026778 | 582 | 267 | 1.1441 | 0.363689952 | 0.679274184 | yes |
| TCONS_L2_00026779 | 1167 | 267 | 1.1441 | 0.363689952 | 0.671486925 | yes |
| TCONS_L2_00026780 | 659 | 135 | 0.3685 | −0.40494765 | 0.00027646 | no |
| TCONS_L2_00026781 | 400 | 156 | 0.6087 | −0.431469675 | 0.000628962 | no |
| TCONS_L2_00026782 | 255 | 246 | 0.4729 | −0.16219555 | 0.006446424 | no |
| TCONS_L2_00026783 | 287 | 204 | 0.9136 | 0.316628344 | 0.279341985 | no |
| TCONS_L2_00026784 | 1549 | 210 | 1.0844 | 0.305066245 | 0.377734015 | yes |
| TCONS_L2_00027441 | 929 | 426 | 0.4914 | 0.202855179 | 0.339790484 | no |
| Protein coding gene controls | | | | | | |
| GAPDH | | | | | | |
| ENST00000229239.5 | 1875 | 1008 | 1.2926 | 0.519960341 | 0.999962146 | yes |
| ENST00000396861.1 | 1348 | 1008 | 1.2926 | 0.519960341 | 0.999963338 | yes |
| ENST00000396858.1 | 1292 | 882 | 1.2952 | 0.533919114 | 0.999870952 | yes |
| ENST00000396859.1 | 1256 | 1008 | 1.2926 | 0.519960341 | 0.999963542 | yes |
| ENST00000396856.1 | 1266 | 783 | 1.315 | 0.548545855 | 0.999679465 | yes |
| GUSB | | | | | | |
| ENST00000304895.4 | 2300 | 339 | 1.2668 | 0.277977644 | 0.776289336 | yes |
| ENST00000421103.1 | 1742 | 339 | 1.2668 | 0.277977644 | 0.782118123 | yes |
| ENST00000345660.6 | 2027 | 285 | 1.1446 | 0.333726267 | 0.659495687 | yes |

* Reference: CPAT: Coding-Potential Assessment Tool using an alignment-free logistic regression model. Nucleic Acid Research.

TABLE 5B
iSeeRNA Results.

| | | NONCODING |
|---|---|---|
| ID | C/NC | SCORE |
| PCAN-1 (XLOC_002996) | | |
| TCONS_00007074 | noncoding | 0.9837 |
| TCONS_00007073 | noncoding | 0.996 |
| TCONS_00006377 | noncoding | 0.9873 |
| TCONS_00006378 | noncoding | 0.9828 |
| PCAN-2 (XLOC_l2_004121) | | |
| TCONS_l2_00008295 | noncoding | 0.8142 |
| TCONS_l2_00008296 | noncoding | 0.7723 |
| TCONS_l2_00008290 | noncoding | 0.9384 |
| TCONS_l2_00008289 | noncoding | 0.9267 |
| TCONS_l2_00008292 | noncoding | 0.9812 |
| TCONS_l2_00008294 | noncoding | 0.9769 |
| TCONS_l2_00008291 | noncoding | 0.9582 |
| TCONS_l2_00008293 | noncoding | 0.9681 |
| PCAN-3 (XLOC_l2_004340) | | |
| TCONS_l2_00008348 | noncoding | 0.9764 |
| TCONS_l2_00008347 | noncoding | 0.9819 |
| TCONS_l2_00008343 | noncoding | 0.8167 |
| TCONS_l2_00008346 | noncoding | 0.9733 |
| TCONS_l2_00007970 | noncoding | 0.9729 |
| PCAN-4 (XLOC_l2_007509) | | |
| TCONS_l2_00013919 | coding | 0.208638 |
| TCONS_l2_00013920 | noncoding | 0.9405 |
| TCONS_l2_00013918 | noncoding | 0.6351 |
| PCAN-5 (XLOC_l2_009441) | | |
| TCONS_l2_00017847 | noncoding | 0.6722 |
| TCONS_l2_00017848 | noncoding | 0.9813 |
| TCONS_l2_00017849 | noncoding | 0.9852 |
| TCONS_l2_00017850 | noncoding | 0.9341 |
| TCONS_l2_00017851 | noncoding | 0.9674 |
| TCONS_l2_00017852 | noncoding | 0.8795 |
| TCONS_l2_00017853 | noncoding | 0.984 |
| TCONS_l2_00018290 | noncoding | 0.8125 |
| TCONS_l2_00018291 | noncoding | 0.9676 |
| TCONS_l2_00018292 | noncoding | 0.9702 |
| TCONS_l2_00018293 | noncoding | 0.9803 |
| TCONS_l2_00018294 | noncoding | 0.9135 |
| TCONS_l2_00018295 | noncoding | 0.9988 |
| TCONS_l2_00018296 | noncoding | 0.9808 |
| PCAN-6 (XLOC_l2_013931) | | |
| TCONS_l2_00026774 | noncoding | 0.6841 |
| TCONS_l2_00026769 | coding | 0.0722 |
| TCONS_l2_00026766 | coding | 0.2569 |
| TCONS_l2_00026779 | noncoding | 0.8778 |
| TCONS_l2_00026780 | noncoding | 0.8494 |
| TCONS_l2_00026781 | noncoding | 0.9237 |
| TCONS_l2_00026767 | noncoding | 0.7907 |
| TCONS_l2_00026771 | noncoding | 0.872 |
| TCONS_l2_00026778 | noncoding | 0.6522 |
| TCONS_l2_00026776 | noncoding | 0.7908 |
| TCONS_l2_00026777 | noncoding | 0.605 |
| TCONS_l2_00027441 | coding | 0.141 |
| TCONS_l2_00026784 | noncoding | 0.8035 |
| TCONS_l2_00026768 | noncoding | 0.8508 |
| TCONS_l2_00026773 | noncoding | 0.8681 |
| TCONS_l2_00026770 | noncoding | 0.6034 |
| TCONS_l2_00026783 | noncoding | 0.7266 |
| TCONS_l2_00026782 | noncoding | 0.5388 |
| TCONS_l2_00026775 | noncoding | 0.8405 |
| Protein coding gene controls | | |
| GAPDH | | |
| ENST00000229239.5 | coding | 0 |
| ENST00000396861.1 | coding | 0 |
| ENST00000396858.1 | coding | 0.002 |
| ENST00000396859.1 | coding | 0 |
| ENST00000396856.1 | coding | 0.0038 |
| GUSB | | |
| ENST00000304895.4 | coding | 0 |
| ENST00000421103.1 | coding | 0 |
| ENST00000345660.6 | coding | 0 |

TABLE 6
Classification performance metrics of the lincRNA diagnostic model across all datasets (TCGA_training,
TCGA_testing, GSE58135_breast_cancer, GSE50760_colon_cancer and GSE25599_liver_cancer). Metrics
used are Area Under the Curve (AUC), Accuracy, F-score, Matthew's correlation coefficient (MCC), Sensitivity, Specificity and Precision.

| | | | | | |
|---|---|---|---|---|---|
| | | | | | Our |
| | | | | | BRCA |
| TCGA_training | TCGA_testing | GSE58135_breast_cancer | GSE50760_colon_cancer | GSE25599_liver_cancer | dataset |

Area under curve

| | | | | | | |
|---|---|---|---|---|---|---|
| GSVM | 0.946 | 0.939 | 0.545 | 0.827 | 0.910 | 0.750 |
| LSVM | 0.918 | 0.917 | 0.968 | 0.796 | 0.885 | 1.000 |
| Random | 0.947 | 0.942 | 0.972 | 0.841 | 0.970 | 0.950 |
| Forest | | | | | | |
| Logistic | 0.901 | 0.905 | 0.646 | 0.410 | 0.390 | 0.550 |
| Regression | | | | | | |

Max accuracy

| | | | | | | |
|---|---|---|---|---|---|---|
| GSVM | 0.894 | 0.903 | 0.743 | 0.861 | 0.850 | 0.773 |
| LSVM | 0.856 | 0.851 | 0.921 | 0.833 | 0.850 | 1.000 |
| Random | 0.887 | 0.895 | 0.936 | 0.861 | 0.900 | 0.909 |
| Forest | | | | | | |
| Logistic | 0.828 | 0.831 | 0.679 | 0.639 | 0.600 | 0.636 |
| Regression | | | | | | |

F-score

| | | | | | | |
|---|---|---|---|---|---|---|
| GSVM | 0.788 | 0.808 | 0.489 | 0.732 | 0.734 | 0.624 |
| LSVM | 0.712 | 0.708 | 0.836 | 0.684 | 0.734 | 1.000 |
| Random | 0.775 | 0.790 | 0.873 | 0.732 | 0.816 | 0.817 |
| Forest | | | | | | |
| Logistic | 0.658 | 0.665 | 0.418 | 0.302 | 0.250 | 0.346 |
| Regression | | | | | | |

Matthew's correlation coeficient

| | | | | | | |
|---|---|---|---|---|---|---|
| GSVM | 0.894 | 0.897 | 0.822 | 0.848 | 0.870 | 0.800 |
| LSVM | 0.855 | 0.843 | 0.936 | 0.824 | 0.842 | 1.000 |
| Random | 0.888 | 0.890 | 0.944 | 0.848 | 0.909 | 0.900 |
| Forest | | | | | | |
| Logistic | 0.826 | 0.835 | 0.750 | 0.667 | 0.667 | 0.645 |
| Regression | | | | | | |

### Example 2. Novel lincRNAs as Pan-Cancer Biomarkers

The emergence of high-throughput sequencing techniques has transformed our understanding of how the genome is regulated by revealing novel transcripts associated with the cancer phenotype. Our group has recently identified and reported a panel of 6 previously unannotated long intergenic non-coding RNAs (lincRNA), namely PCAN1 to PCAN-6, as pan-cancer biomarkers (Example 1; Ching T, et al. 2016, 7:62-72, EBioMedicine). These PCANs were differentially expressed in over 1200 tumor and tumor adjacent normal tissues, from 10 cancer types in TCGA RNA-Seq datasets. Obtained from primary tumor tissues, they are highly accurate (AUC=0.95) and highly robust pan-cancer biomarkers that were validated in over 3300 samples from 5 different cohorts. It was further demonstrated that these pan-cancer lincRNAs are biologically functional (one of the five criteria for biomarker to be approved by FDA), using cell culture experiments. As described herein, experiments to investigate these novel pan-cancer molecules as blood based biomarkers in Prostate, Lung, Colorectal, and Ovarian (PLCO) samples are performed. Specifically, the PCAN lincRNA occurrence and abundance in 50 plasma samples of each PLCO cancer type, in comparison to the age and gender matched healthy control plasma samples, are investigated. The PCAN expression data is correlated with patient clinical information to better calibrate the accuracy of biomarker panel, as well as the patient prognosis. In summary, lincRNAs may be an efficient and cost effective pan-cancer screening biomarkers.

### Specific Aims:

lincRNAs as pan-cancer biomarkers are investigated in 200 PLCO vs. 200 healthy control plasma samples (50 cases vs. 50 controls for each of the PLCO cancer types), as an extended study from our previous obtained solid results on tissue-based lincRNA biomarkers (Ching T, et al. 2016, 7:62-72, EBioMedicine). This investigation focuses on three specific aims.

1) Determine the Occurrence and Abundance of the Six PCAN lincRNAs in PLCO Plasma Samples.

In this Aim total RNA is isolated from matched normal and cancer plasma samples and the expression of PCAN lincRNAs is determined.

2) Correlate PCAN Expression with Patient Prognosis and Other Clinical Information.

In this Aim computational analysis is used to determine the relationship between PCAN lincRNA expression and patient clinical information, such as survival, age, gender and tumor stage. A new model is constructed that predicts the cancer risks based on PCAN expression and other clinical information, similar to previous reports (Huang et al., Cancer Epidemiology, Biomarkers and Prevention, 2016 Jul. 6. pii: cebp.0260; Huang et al., PLOS Computational Biology. September 18; 10(9):e1003851).

3) Perform Untargeted lincRNA-Seq Experiments in PLCO Samples.

Untargeted lincRNA-Seq experiments in plasma samples is also performed to detect new lincRNA biomarker candidates in the plasma.

All publications, patents, and patent documents are incorporated by reference herein, as though individually incorporated by reference, including the following documents discussed throughout the specification:
Berrar, D., Bradbury, I., Bubitzky, W., 2006. Avoiding model selection bias in small-sample genomic datasets. Bioinformatics 22, 1245-1250.
BROAD, 2014. Broad Institute TCGA Genome Data Analysis Center (2014): Analysis Overview for 15 Jul. 2014. Broad Institute of MIT and Harvard.
Brockdorff, N., Ashworth, A., Kay, G. F., Cooper, P., Smith, S., Mccabe, V. M., Norris, D. P., Penny, G. D., Patel, D., Rastan, S., 1991. Conservation of position and exclusive expression of mouse xist from the inactive X chromosome. Nature 351, 329-331.
Cabili, M. N., Trapnell, C., Goff, L., Koziol, M., Tazon-Vega, B., Regev, A., Rinn, J. L., 2011. Integrative annotation of human large intergenic noncoding RNAs reveals global properties and specific subclasses. Genes Dev. 25, 1915-1927.
Camacho, C., Coulouris, G., Avagyan, V., Ma, N., Papadopoulos, J., Bealer, K., Madden, T. L., 2009. BLAST+: architecture and applications. BMC Bioinf. 10, 421.
Cancer Genome Atlas, N., 2012. Comprehensive molecular portraits of human breast tumours. Nature 490, 61-70.
Cancer Genome Atlas Research, N., Weinstein, J. N., Collisson, E. A., Mills, G. B., Shaw, K. R., Ozenberger, B. A., Ellrott, K., Shmulevich, I., Sander, C., Stuart, J. M., 2013. The cancer genome atlas Pan-cancer analysis project. Nat. Genet. 45, 1113-1120.
Ching, T., Huang, S., Garmire, L. X., 2014. Power analysis and sample size estimation for RNA-seq differential expression. RNA 20, 1684-1696.
Ching, et al., EBioMedicine 7:62-72 (2016).
Consortium, E. P., 2012. An integrated encyclopedia of DNA elements in the human genome. Nature 489, 57-74.
Du, Z., Fei, T., Verhaak, R. G., Su, Z., Zhang, Y., Brown, M., Chen, Y., Liu, X. S., 2013. Integrative genomic analyses reveal clinically relevant long noncoding RNAs in human cancer. Nat. Struct. Mol. Biol. 20, 908-913.
Garmire, L. X., Garmire, D. G., Huang, W., Yao, J., Glass, C. K., Subramaniam, S., 2011. A global clustering algorithm to identify long intergenic non-coding RNA—with applications in mouse macrophages. PLoS One 6, e24051.
Ge, X., Chen, Y., Liao, X., Liu, D., Li, F., Ruan, H., Jia, W., 2013. Overexpression of long noncoding RNA PCAT-1 is a novel biomarker of poor prognosis in patients with colorectal cancer. Med. Oncol. 30, 1-6.
Gupta, R. A., Shah, N., Wang, K. C., Kim, J., Horlings, H. M., Wong, D. J., Tsai, M.-C., Hung, T., Argani, P., Rinn, J. L., 2010. Long non-coding RNA HOTAIR reprograms chromatin state to promote cancer metastasis. Nature 464, 1071-1076.
Habel, L. A., Shak, S., Jacobs, M. K., Capra, A., Alexander, C., Pho, M., Baker, J., Walker, M., Watson, D., Hackett, J., 2006. A population-based study of tumor gene expression and risk of breast cancer death among lymph node-negative patients. Breast Cancer Res. 8, R25.
Han, L., Yuan, Y., Zheng, S., Yang, Y., Li, J., Edgerton, M. E., Diao, L., Xu, Y., Verhaak, R. G., Liang, H., 2014. The Pan-cancer analysis of pseudogene expression reveals biologically and clinically relevant tumour subtypes. Nat. Commun. 5.
Huang, S., Yee, C., Ching, T., Yu, H., Garmire, L. X., 2014. A novel model to combine clinical and pathway-based transcriptomic information for the prognosis prediction of breast cancer. PLoS Comput. Biol. 10, e1003851.
Ji, P., Diederichs, S., Wang, W., Bing, S., Metzger, R., Schneider, P. M., Tidow, N., Brandt, B., Buerger, H., Bulk, E., 2003. MALAT-1, a novel noncoding RNA, and thymosin (34 predict metastasis and survival in early-stage non-small cell lung cancer. Oncogene 22, 8031-8041.
Iyer, M. K., Niknafs, Y. S., Malik, R., Singhal, U., Sahu, A., Hosono, Y., Barrette, T. R., Prensner, J. R., Evans, J. R., Zhao, S., 2015. The landscape of long noncoding RNAs in the human transcriptome. Nat. Genet. 47 (3), 199-208.
Kandoth, C., Mclellan, M. D., Vandin, F., Ye, K., Niu, B., Lu, C., Xie, M., Zhang, Q., Mcmichael, J. F., Wyczalkowski, M. A., Leiserson, M. D., Miller, C. A., Welch, J. S., Walter, M. J., Wendl, M. C., Ley, T. J., Wilson, R. K., Raphael, B. J., Ding, L., 2013. Mutational landscape and significance across 12 major cancer types. Nature 502, 333-339.
Khalil, A. M., Guttman, M., Huarte, M., Garber, M., Raj, A., Morales, D. R., Thomas, K., Presser, A., Bernstein, B. E., Van Oudenaarden, A., 2009. Many human large intergenic noncoding RNAs associate with chromatin-modifying complexes and affect gene expression. Proc. Natl. Acad. Sci. 106, 11667-11672.
Kowalczyk, M. S., Higgs, D. R., Gingeras, T. R., 2012. Molecular biology: RNA discrimination. Nature 482, 310-311.
Liang, C. C., Park, A. Y., Guan, J. L., 2007. In vitro scratch assay: a convenient and inexpensive method for analysis of cell migration in vitro. Nat. Protoc. 2, 329-333.
Liao, Q., Liu, C., Yuan, X., Kang, S., Miao, R., Xiao, H., Zhao, G., Luo, H., Bu, D., Zhao, H., 2011.
Large-scale prediction of long non-coding RNA functions in a coding-non-coding gene co-expression network. Nucleic Acids Res. 39, 3864-3878.
Liao, Y., Smyth, G., Shi, W., 2013. featureCounts: an efficient general-purpose read summarization program. (arXiv, 1305, 16).
Liao, Y., Smyth, G. K., Shi, W., 2014. featureCounts: an efficient general purpose program for assigning sequence reads to genomic features. Bioinformatics 30, 923-930.
Ling, H., Spizzo, R., Atlasi, Y., Nicoloso, M., Shimizu, M., Redis, R. S., Nishida, N., Gafi, R., Song, J., Guo, Z., 2013. CCAT2, a novel noncoding RNA mapping to 8q24, underlies metastatic progression and chromosomal instability in colon cancer. Genome Res. 23, 1446-1461.
Liu, K., Yan, Z., Li, Y., Sun, Z., 2013. Linc2GO: a human LincRNA function annotation resource based on ceRNA hypothesis. Bioinformatics 29, 2221-2222.
Livak, K. J., Schmittgen, T. D., 2001. Analysis of relative gene expression data using realtime quantitative PCR and the 2-ΔΔCT method. Methods 25, 402-408.
Love, M., Anders, S., Huber, W., 2013. Differential Analysis of RNA-Seq Data at the Gene Level Using the DESeq2 Package.
Love, M. I., Huber, W., Anders, S., 2014. Moderated estimation of fold change and dispersion for RNA-Seq data with DESeq2. (bioRxiv).
Ma, H., Hao, Y., Dong, X., Gong, Q., Chen, J., Zhang, J., Tian, W., 2012. Molecular mechanisms and function prediction of long noncoding RNA. Sci. World J. 2012.
Mchugh, C. A., Russell, P., Guttman, M., 2014. Methods for comprehensive experimental identification of RNA-protein interactions. Genome Biol. 15, 203.
Menor, M., Ching, T., Zhu, X., Garmire, D., Garmire, L. X., 2014. mirMark: a site-level and UTR-level classifier for miRNA target prediction. Genome Biol. 15, 500.
Penny, G. D., Kay, G. F., Sheardown, S. A., Rastan, S., Brockdorff, N., 1996. Requirement for xist in X chromosome inactivation. Nature 379, 131-137.
Prensner, J. R., Iyer, M. K., Balbin, O. A., Dhanasekaran, S. M., Cao, Q., Brenner, J. C., Laxman, B., Asangani, I. A., Grasso, C. S., Kominsky, H. D., 2011. Transcriptome sequencing across a prostate cancer cohort identifies PCAT-1, an unannotated lincRNA implicated in disease progression. Nat. Biotechnol. 29, 742-749.
Rinn, J. L., Kertesz, M., Wang, J. K., Squazzo, S. L., Xu, X., Brugmann, S. A., Goodnough, L. H., Helms, J. A., Farnham, P. J., Segal, E., 2007. Functional demarcation of active and silent chromatin domains in human b i N HOXb/i N loci by noncoding RNAs. Cell 129, 1311-1323.
Rubie, C., Kempf, K., Hans, J., Su, T., Tilton, B., Georg, T., Brittner, B., Ludwig, B., Schilling, M., 2005. Housekeeping gene variability in normal and cancerous colorectal, pancreatic, esophageal, gastric and hepatic tissues. Mol. Cell. Probes 19, 101-109.
Salmena, L., Poliseno, L., Tay, Y., Kats, L., Pandolfi, P. P., 2011. A b i N ceRNAb/i N hypothesis: the Rosetta Stone of a hidden RNA Language? Cell 146, 353-358.
Sun, K., Chen, X., Jiang, P., Song, X., Wang, H., Sun, H., 2013. iSeeRNA: identification of long intergenic non-coding RNA transcripts from transcriptome sequencing data. BMC Genomics 14, S7.
Trapnell, C., Williams, B. A., Pertea, G., Mortazavi, A., Kwan, G., Van Baren, M. J., Salzberg, S. L., Wold, B. J., Pachter, L., 2010. Transcript assembly and quantification by RNA-seq reveals unannotated transcripts and isoform switching during cell differentiation. Nat. Biotechnol. 28, 511-515.
Tripathi, V., Ellis, J. D., Shen, Z., Song, D. Y., Pan, Q., Watt, A. T., Freier, S. M., Bennett, C. F., Sharma, A., Bubulya, P. A., 2010. The nuclear-retained noncoding RNA MALAT1 regulates alternative splicing by modulating SR splicing factor phosphorylation. Mol. Cell 39, 925-938.
Ulitsky, I., Bartel, D. P., 2013. lincRNAs: genomics, evolution, and mechanisms. Cell 154, 26-46.
Vitiello, M., Tuccoli, A., Poliseno, L., 2014. Long non-coding RNAs in cancer: implications for personalized therapy. Cell. Oncol. 1-12.
Volinia, S., Croce, C. M., 2013. Prognostic microRNA/mRNA signature from the integrated analysis of patients with invasive breast cancer. Proc. Natl. Acad. Sci. U.S.A 110, 7413-7417.
Wang, K., Singh, D., Zeng, Z., Coleman, S. J., Huang, Y., Savich, G. L., He, X., Mieczkowski, P., Grimm, S. A., Perou, C. M., 2010. MapSplice: accurate mapping of RNA-seq reads for splice junction discovery. Nucleic Acids Res. gkq622.
Wang, L., Park, H. J., Dasari, S., Wang, S., Kocher, J.-P., Li, W., 2013. CPAT: coding-potential assessment tool using an alignment-free logistic regression model. Nucleic Acids Res. 41, e74-e74.
Weakley, S. M., Wang, H., Yao, Q., Chen, C., 2011. Expression and function of a large noncoding RNA Gene XIST in human cancer. World J. Surg. 35, 1751-1756.
Weinstein, J. N., Collisson, E. A., Mills, G. B., Shaw, K. R. M., Ozenberger, B. A., Ellrott, K., Shmulevich, I., Sander, C., Stuart, J. M., Network, C. G. A. R., 2013. The cancer genome atlas pan-cancer analysis project. Nat. Genet. 45, 1113-1120.
Wilks, C., Cline, M. S., Weiler, E., Diehkans, M., Craft, B., Martin, C., Murphy, D., Pierce, H., Black, J., Nelson, D., 2014. The Cancer Genomics Hub (CGHub): overcoming cancer through the power of torrential data. Database 2014, bau093.
Yuan, J., Wu, W., Xie, C., Zhao, G., Zhao, Y., Chen, R., 2014. NPInter v2.0: an updated database of ncRNA interactions. Nucleic Acids Res. 42, D104-D108.

The invention has been described with reference to various specific and preferred embodiments and techniques. However, it should be understood that many variations and modifications may be made while remaining within the spirit and scope of the invention.

1-2. (canceled)

3. A method for identifying a patient having cancer, comprising detecting increased expression of PCAN-1 in a nucleic acid sample that was derived from a biological sample obtained from the patient, wherein increased expression of PCAN-1, as compared to expression from a control sample, indicates the patient has cancer.

4-6. (canceled)

7. A method comprising:
1) detecting increased expression of PCAN-1 in a nucleic acid sample that was derived from a biological sample obtained from a patient;
2) diagnosing the patient with cancer when increased expression of PCAN-1 is detected, as compared to expression from a control sample; and
3) administering an effective amount of a therapeutic agent to the patient.

8-10. (canceled)

11. The method of claim 3, wherein expression of PCAN-1 is increased by at least about 10%.

12. The method of claim 7, wherein expression of PCAN-1 is increased by at least about 10%.

13-19. (canceled)

20. A method for treating cancer in a patient comprising administering an effective amount of a therapeutic agent to the patient, wherein the cancer was determined to comprise increased expression of PCAN-1, as compared to expression from a control.

21-28. (canceled)

29. The method of claim 20, wherein expression of PCAN-1 was increased by at least about 10%.

30-34. (canceled)

35. The method of claim 3, wherein the cancer is a breast, head and neck, thyroid, colon, kidney, liver, lung, prostate, gastric, ovarian or endometrial cancer.

36-37. (canceled)

38. The method of claim 3, wherein the PCAN-1 expression is detected using reverse transcriptase-polymerase chain reaction (RT-PCR) methods, quantitative real-time PCR (qPCR), microarray, RNA sequencing (RNA-Seq), next generation RNA sequencing (deep sequencing), gene expression analysis by massively parallel signature sequencing (MPSS), or transcriptomics.

39-43. (canceled)

44. The method of claim 7, wherein the therapeutic agent is an anti-cancer agent.

45. The method of claim 7, wherein the therapeutic agent is a chemotherapeutic agent.

46-49. (canceled)

50. The method of claim 7, wherein the therapeutic agent is an antisense nucleic acid selected from the group consisting of siRNA, shRNA, or miRNA.

51-57. (canceled)

58. The method of claim 7, wherein the biological sample is a tissue sample or a plasma sample.

59-64. (canceled)

65. The method of claim 7, wherein the cancer is a breast, head and neck, thyroid, colon, kidney, liver, lung, prostate, gastric, ovarian or endometrial cancer.

66. The method of claim 7, wherein the cancer is breast cancer or lung cancer.

67. The method of claim 7, wherein the PCAN-1 expression is detected using reverse transcriptase-polymerase chain reaction (RT-PCR) methods, quantitative real-time PCR (qPCR), microarray, RNA sequencing (RNA-Seq), next generation RNA sequencing (deep sequencing), gene expression analysis by massively parallel signature sequencing (MPSS), or transcriptomics.

68. The method of claim 20, wherein the cancer is a breast, head and neck, thyroid, colon, kidney, liver, lung, prostate, gastric, ovarian or endometrial cancer.

69. The method of claim 20, wherein the cancer is breast cancer or lung cancer.

70. The method of claim 20, wherein the therapeutic agent is an anti-cancer agent.

71. The method of claim 20, wherein the therapeutic agent is an antisense nucleic acid selected from the group consisting of siRNA, shRNA, or miRNA.
